(19) United States
(12) Patent Application Publication
(10) Pub. No.: US 2024/0246957 A1
(43) Pub. Date: Jul. 25, 2024

(54) Diazepane Derivatives, Processes for their Preparation, and Uses thereof for the Amelioration, Prevention and/or Treatment of Mental and Neurological Diseases

(71) Applicant: 2N Pharma ApS, Aalborg (DK)

(72) Inventors: Venkateswara Rao BODDU, Aalborg (DK); Santhosh Kumar CHITTIMALLA, Aalborg (DK); Durairaja SANKARAN, Aalborg (DK); Ganesh Babu KARUNAKARAN, Aalborg (DK); Thomas BRIMERT, Aalborg (DK)

(21) Appl. No.: 18/558,763

(22) PCT Filed: May 4, 2022
(86) PCT No.: PCT/EP2022/061994

§ 371 Date: Nov. 3, 2023

(30) Foreign Application Priority Data
May 4, 2021 (EP) 21172027.1

(51) Int. Cl. C07D 417/14; A61K 31/551; A61P 25/28; C07D 417/04

(52) U.S. Cl. CPC C07D 417/14; A61K 31/551; A61P 25/28; C07D 417/04

(57) ABSTRACT

Compounds of the formula (I)

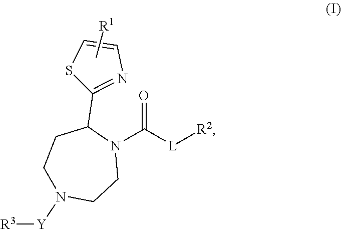

wherein R¹, R², R³, L and Y have the designations described herein, or a pharmaceutically or veterinarily acceptable salt, hydrate or solvate thereof. Further, the invention relates to processes for the preparation of compounds of the formula (I) or a pharmaceutically or veterinarily acceptable salt, hydrate or solvate thereof. The invention also relates to compounds of the formula (I) or a pharmaceutically or veterinarily acceptable salt, hydrate or solvate thereof for use as a medicament. Further, the invention relates to compounds of the formula (1) or a pharmaceutically or veterinarily acceptable salt, hydrate or solvate thereof for use in the amelioration, prevention and/or treatment of a disease caused by or related to delipidation of a neural tissue. In particular, the disease is a neurodegenerative disease.

### FIELD OF THE INVENTION

The present invention relates to compounds of the formula (I)

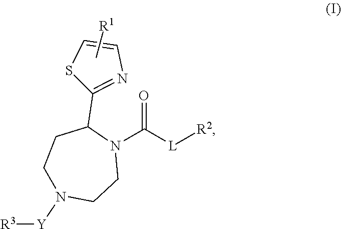

wherein R¹, R², R³, L and Y have the designations described herein, or a pharmaceutically or veterinarily acceptable salt, hydrate or solvate thereof. Further, the invention relates to processes for the preparation of compounds of the formula (I) or a pharmaceutically or veterinarily acceptable salt, hydrate or solvate thereof. The invention also relates to compounds of the formula (I) or a pharmaceutically or veterinarily acceptable salt, hydrate or solvate thereof for use as a medicament. Further, the invention relates to compounds of the formula (I) or a pharmaceutically or veterinarily acceptable salt, hydrate or solvate thereof for use in the amelioration, prevention and/or treatment of a disease caused by or related to delipidation of a neural tissue. In particular, the disease is a neurodegenerative disease.

### BACKGROUND OF THE INVENTION

Mental and neurological diseases remain a public health problem worldwide. A mental disease is a behavioral or mental pattern that causes significant distress or impairment of personal functioning. Examples of mental diseases include depression and impairment of recent and remote memory (loss of short and long-term memory). For example, major depressive disorder is a common and complex disease with prolonged periods of suppressed mood and loss of interest in all or almost all activities.

Neurological diseases are diseases that affect the central nervous system (CNS) or the peripheral nervous systems and can impair the brain, spinal cord, peripheral nerve or neuromuscular function. In the last decades, the incidence of central nervous (CNS) diseases including multiple sclerosis (MS), amyotrophic lateral sclerosis (ALS) and Parkinson's Disease (PD) have been increasing. CNS diseases, specifically neurodegenerative diseases, have a significant impact on the quality of life and represent a major burden to both close relatives and society at large. Up to today, only a few treatment options exist, but cure is not yet available.

It has been found that several mental and neurological diseases are caused by or related to delipidation of neural tissue, in particular to delipidation of myelin sheets. It has been suggested that several mental and neurological diseases can be treated or prevented by inhibiting the enzyme Carnitine-Palmitoyl-Transferase-1 (CPT-1) pharmacologically or genetically (Scientific Reports, 9, 13299, 1-11, Sep. 16, 2019). A. Skottrup Morkholt et al. (Scientific Reports, 7, 2158, 1-9, May 19, 2017) found that blocking of CPT-1 reduces stress-induced depression in rat. Further, M. S. Trabjerg et al. (Scientific Reports, 10, 15583, 1-19, 2020 and Nature Communications, 4, 509, 1-23, Apr. 30, 2021) show that by downregulating the metabolism of lipids through CPT-1, it is possible to reverse or slowdown disease progression of several CNS diseases in experimental models.

Lipids have several functions in the CNS. One of the key functions of lipids is to build and maintain the myelin sheath on the axons of neurons. In the multiple sclerosis (MS) lesions, CPT-1 expression is greatly increased, which correlates with a decrease in lipid concentration in the myelin sheath due to an increased beta-oxidation. In Parkinson's Disease (PD), several mechanisms leading to neurodegeneration such as mitochondrial dysfunction, and oxidative stress are linked to the metabolism of lipids. In amyotrophic lateral sclerosis (ALS), a decrease in lipid level could lead to disease progression and lipid metabolism seems to be upregulated prior to disease.

WO 2009/156479 A1 describes arylalkyl- and aryloxyalkyl-substituted oxirane carboxylic acid derivatives as CPT-1 inhibitors for use in treating and/or preventing disorders caused by delipidation of neural tissue. No human data, tests in oral administration, or specific dosages indicating the treatment are described.

WO 2007/063012 A1 describes heteroaryl substituted piperidine derivatives for use as therapeutic active substances for the treatment and/or prophylaxis of diseases, which are modulated by L-CPT1 inhibitors.

WO 2018/122254 A1 describes etomoxir with the chemical formula 2-[6-(4-chlorophenoxy) hexyl]-oxirane-2-carboxylic acid for use in the treatment, prevention and/or amelioration of brain diseases caused by delipidation of neural tissue.

As mentioned above, compounds that are suitable for the effective prevention and/or treatment of mental and neurological diseases have not yet been found.

Thus, there is a need for novel compounds, which specifically inhibit Carnitine-Palmitoyl-Transferase-1 (CPT-1) and that are suitable for use as a medicament. In particular, novel compounds are of interest that can be used in the amelioration, prevention and/or treatment of mental and neurological diseases, in particular of multiple sclerosis (MD), autoimmune encephalomyelitis, Parkinson's Disease (PD), and amyotrophic lateral sclerosis (ALS).

### SUMMARY OF THE INVENTION

The present invention, in one aspect, relates to a compound of the formula (I)

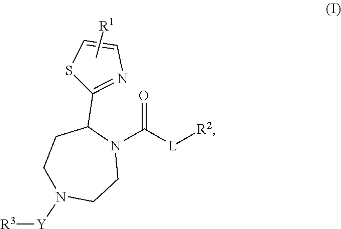

wherein
R¹ is unsubstituted or substituted aryl, preferably unsubstituted or substituted phenyl, naphthyl, tetrahydronaphthyl, indenyl, indanyl, pentalenyl, or fluorenyl,
   unsubstituted or substituted heteroaryl, preferably unsubstituted or substituted pyridyl, pyrazinyl, pyrimidinyl, pyridazinyl, 2-oxo-1,2-dihydropyridinyl, oxazolyl, oxydiazolyl, isoxazolyl, thiadiazolyl, tetrazolyl, pyrazolyl, imidazolyl, thiazolyl and thienyl, quinolinyl, isoquinolinyl, cinnolinyl, pyrazolo[1,5-a]pyridyl, imidazo[1,2-a]pyridyl, quinoxalinyl, benzothiazolyl, benzotriazolyl, indolyl, or indazolyl,
   unsubstituted or substituted 5- or 6-membered saturated or partially unsaturated heterocyclyl, or
   unsubstituted or substituted C₃-C₈-cycloalkyl, or cyclohexenyl;
L is a single bond, or a difunctional linker, preferably *—O—, *—OCH₂—, *—CH₂O—, *—CH₂—, *—CH₂—CH₂—, *—CH₂—CH₂—CH₂—, or *—CH₂—C(CH₃)₂—, or a trifunctional linker, preferably, *—CH═, wherein the * indicates the point of attachment to the carbonyl (C═O) group;
R² is unsubstituted or substituted phenyl, naphthyl, or pyridyl, or C₁-C₄-alkyl;
R³ is H, C₁-C₈-alkyl, halogen-C₁-C₄-alkyl, or C₃-C₈-cycloalkyl,
   unsubstituted or substituted 4, 5- or 6-membered saturated or partially unsaturated heterocyclyl, or
   unsubstituted or substituted phenyl;
Y is —(C═O)—, —(SO₂)— or a single bond;
or a stereoisomer, a pharmaceutically or veterinarily acceptable salt, hydrate or solvate thereof.

In one embodiment, the invention relates to a compound of the formula (I)

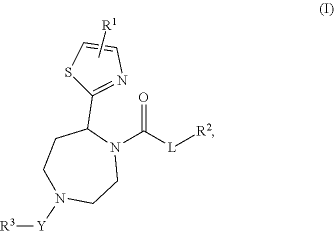

wherein
R¹ is unsubstituted or substituted aryl, preferably unsubstituted or substituted phenyl, naphthyl, tetrahydronaphthyl, indenyl, indanyl, pentalenyl, or fluorenyl,
   unsubstituted or substituted heteroaryl, preferably unsubstituted or substituted pyridyl, pyrazinyl, pyrimidinyl, pyridazinyl, 2-oxo-1,2-dihydropyridinyl, oxazolyl, oxydiazolyl, isoxazolyl, thiadiazolyl, tetrazolyl, pyrazolyl, imidazolyl, thiazolyl and thienyl, quinolinyl, isoquinolinyl, cinnolinyl, pyrazolo[1,5-a]pyridyl, imidazo[1,2-a]pyridyl, quinoxalinyl, benzothiazolyl, benzotriazolyl, indolyl, or indazolyl,
   unsubstituted or substituted 5- or 6-membered saturated or partially unsaturated heterocyclyl, or
   unsubstituted or substituted C₃-C₈-cycloalkyl, or cyclohexenyl;
L is a single bond, or a difunctional linker, preferably *—O—, *—OCH₂—, *—CH₂O—, *—CH₂—, *—CH₂—CH₂—, *—CH₂—CH₂—CH₂—, or *—CH₂—C(CH₃)₂—, wherein the * indicates the point of attachment to the carbonyl (C═O) group;
R² is unsubstituted or substituted phenyl, naphthyl, or pyridyl,
R³ is H, C₁-C₈-alkyl, halogen-C₁-C₄-alkyl, or C₃-C₈-cycloalkyl,
   unsubstituted or substituted 4, 5- or 6-membered saturated or partially unsaturated heterocyclyl, or
   unsubstituted or substituted phenyl;
Y is —(C═O)—, —(SO₂)— or a single bond;
or a stereoisomer, a pharmaceutically or veterinarily acceptable salt, hydrate or solvate thereof.

In one embodiment, the invention relates to a compound of the formula (I) as defined herein, wherein
R¹ is unsubstituted or substituted phenyl, naphthyl, or tetrahydronaphthyl,
   unsubstituted or substituted pyridyl, pyrazinyl, pyrimidinyl, oxazolyl, oxydiazolyl, isoxazolyl, thiadiazolyl, tetrazolyl, pyrazolyl, imidazolyl, thiazolyl, thienyl, or quinolinyl,
   unsubstituted or substituted pyrrolidinyl, piperidinyl, tetrahydropiperidinyl, or piperazinyl, or
   unsubstituted or substituted cyclopentyl; cyclohexyl or cyclohexenyl;
L is a single bond, or a difunctional linker, preferably *—O—, *—OCH₂—, *—CH₂O—, *—CH₂—, or *—CH₂—CH₂—, wherein the * indicates the point of attachment to the carbonyl (C═O) group;
R² is unsubstituted or substituted phenyl, naphthyl, or pyridyl;
R³ is H, C₁-C₄-alkyl, halogen-C₁-C₄-alkyl, cyclopropyl, cyclobutyl, cyclopentyl or cyclohexyl,
   unsubstituted or substituted azetidinyl, pyrrolidinyl, piperidinyl, or oxetanyl,

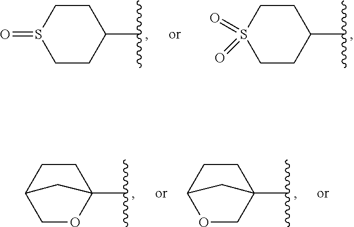 [SMILES: *C1CCS(=O)CC1 ; *C1CCS(=O)(=O)CC1 ; *C12CCC(CO1)C2 ; *C12CCC(C1)OC2]

unsubstituted or substituted phenyl; and
Y is —(C═O)—, —(SO₂)— or a single bond, preferably —(C═O)—.

In another embodiment, the invention relates to a compound of the formula (I) as defined herein, wherein
R¹ is unsubstituted or substituted phenyl, naphthyl, or tetrahydronaphthyl,
   unsubstituted or substituted pyridyl, pyrazinyl, pyrimidinyl, oxazolyl, oxydiazolyl, isoxazolyl, thiadiazolyl, tetrazolyl, pyrazolyl, imidazolyl, thiazolyl, thienyl, or quinolinyl,
   unsubstituted or substituted pyrrolidinyl, piperidinyl, tetrahydropiperidinyl, or piperazinyl, or
   unsubstituted or substituted cyclopentyl, cyclohexyl or cyclohexenyl,
   each R¹ being optionally and independently substituted with one or more, preferably with one of the following residues:
   —CN,
   halogen, preferably —F or —Cl,
   C₁-C₄-alkyl, preferably methyl,
   halogen-C₁-C₄-alkyl, preferably difluoromethyl, or trifluoromethyl,
   SO₂Me, or
   CO₂C₁-C₄-alkyl, preferably CO₂Me.

In another embodiment, the invention relates to a compound of the formula (I) as defined herein, wherein
R¹ is unsubstituted or substituted phenyl,
   unsubstituted or substituted pyridyl, pyrazolyl, thienyl, or quinolinyl,
   unsubstituted or substituted piperidinyl, or tetrahydropiperidinyl, or
   unsubstituted or substituted cyclohexyl or cyclohexenyl;
   each R¹ being optionally and independently substituted with one or more, preferably with one of the following residues:
   —CN,
   —F or —Cl,
   C₁-C₄-alkyl, preferably methyl,
   halogen-C₁-C₄-alkyl, preferably trifluoromethyl,
   SO₂Me, or
   CO₂C₁-C₄-alkyl, preferably CO₂Me.

In another embodiment, the invention relates to a compound of the formula (I) as defined herein, wherein
R¹ is unsubstituted or substituted phenyl or pyridyl,
   being optionally and independently substituted with one or more, preferably with one of the following residues:
   —F or —Cl,
   C₁-C₄-alkyl, preferably methyl,
   trifluoromethyl,
   SO₂Me, or
   CO₂C₁-C₄-alkyl, preferably CO₂Me.

In another embodiment, the invention relates to a compound of the formula (I) as defined herein, wherein
L is a single bond, *—CH₂O—, or *—CH₂—, preferably *—CH₂O—, wherein the * indicates the point of attachment to the carbonyl (C═O) group.

In another embodiment, the invention relates to a compound of the formula (I) as defined herein, wherein
R² is unsubstituted or substituted phenyl, naphthyl, or pyridyl; preferably phenyl,
   each R² being optionally and independently substituted with one or more, preferably with one of the following residues:
   —CN,
   halogen, preferably —F or —Cl,
   C₁-C₄-alkyl, preferably methyl,
   halogen-C₁-C₄-alkyl, preferably difluoromethyl, or trifluoromethyl,
   SO₂Me,
   CO₂C₁-C₄-alkyl, preferably CO₂Me,
   adamantyl,
   unsubstituted or substituted phenyl, being optionally substituted with one or more, preferably with one substituent selected from
   halogen, preferably —F or —Cl,
   halogen-C₁-C₄-alkyl, preferably trifluoromethyl,
   C₃-C₈-cycloalkyl, preferably cyclohexyl, or
   pyridyl.

In another embodiment, the invention relates to a compound of the formula (I) as defined herein, wherein
R³ is H, C₁-C₄-alkyl, halogen-C₁-C₄-alkyl, cyclopropyl, cyclobutyl, cyclopentyl or cyclohexyl,
   unsubstituted or substituted azetidinyl, pyrrolidinyl, piperidinyl, or oxetanyl,
   unsubstituted or substituted phenyl.
   each azetidinyl, pyrrolidinyl, piperidinyl, oxetanyl, or phenyl being optionally and independently substituted with one or more, preferably with one of the following residues:
   —CN,
   halogen, preferably —F or —Cl;
   C₁-C₄-alkyl, preferably methyl;
   halogen-C₁-C₄-alkyl, preferably chloromethyl, dichloromethyl, trichloromethyl, fluoromethyl, difluoromethyl, trifluoromethyl, chlorofluoromethyl, dichlorofluoromethyl, chlorodifluoromethyl, 1-fluoroethyl, 2-fluoroethyl, 2,2-difluoroethyl, 2,2,2-trifluoroethyl, 2-chloro-2fluoroethyl, 2-chloro-2,2-difluoroethyl, 2,2-dichloro-2-fluoroethyl, 2,2,2-trichloroethyl, pentafluoroethyl, or 2,2-difluor-3-methyl-butyl, particularly preferred difluoromethyl, or trifluoromethyl,
   SO₂Me,
   CO₂C₁-C₄-alkyl, preferably CO₂Me, or
   CO—C₁-C₄-alky, preferably CO-Me.

In another embodiment, the invention relates to a compound of the formula (I) as defined herein, wherein
R³ is C₁-C₄-alkyl, preferably methyl.

In another aspect, the invention relates to a compound of the formula (I), as defined herein or a stereoisomer, a pharmaceutically or veterinarily acceptable salt, hydrate or solvate thereof for use as a medicament.

In another aspect, the invention relates to a pharmaceutical composition comprising a compound of formula (I) as defined herein or a stereoisomer, a pharmaceutically or veterinarily acceptable salt, hydrate or solvate thereof, and a therapeutically inert carrier.

In another aspect, the invention relates to a compound of formula (I) as defined herein for use in the amelioration, prevention or treatment of a disease caused by or related to delipidation of a neural tissue, preferably by inhibiting the expression and/or activity of the enzyme Carnitine-Palmitoyl-Transferase-1 (CPT-1).

In another aspect, the invention relates to a compound of formula (I) as defined herein for use in the amelioration, prevention or treatment of a disease which is modulated by CPT-1 inhibitors.

In a preferred embodiment, the disease caused by or related to delipidation of a neural tissue is Morbus Alzheimer, Morbus Parkinson, amyotrophic lateral sclerosis (ALS), inflammatory diseases, acute traumatic events such as surgery or injury, AIDS related wasting due to the toxicity of reverse transcriptase inhibitors, mitochondrial myopathies, senescence and ageing, neuronal ischemia, a polyglutamine disease, dystonia, Leber's heredity optic neuropathy (LHON), schizophrenia, stroke, myodegenerative disorders, Mitochondrial Encephalomyopathy Lactic Acidosis and Strokelike Episodes (MELAS), Myoclonic Epilepsy associated with Ragged-Red Fibers (MERRF), Neuropathy, Ataxia, and Retinitis Pigmentosa (NARP), Progressive External Ophthalmoplegia (PEO), Leigh's disease, Kearns-Sayres Syndromes, muscular dystrophy, myotonic distrophy, chronic fatigue syndrome, Friedreich's Ataxia; developmental delay in cognitive, motor, language, executive function or social skills; epilepsy, peripheral neuropathy, optic neuropathy, autonomic neuropathy, neurogenic bowel dysfunction, sensorineural deafness, neurogenic bladder dysfunction, migraine; renal tubular acidosis, hepatic failure, lactic acidemia, parodontosis, Duchenne muscular dystrophy, Becker's muscular dystrophy, McArdle's disease, abnormities of the testosterone synthesis and/or hypoparathyroidism.

In a particular preferred embodiment, the disease caused by or related to delipidation of a neural tissue is amyotrophic lateral sclerosis (ALS).

In another aspect, the invention relates to a process for manufacturing a compound of formula (I) as described herein.

In one aspect, the invention relates to a compound of the formulae (C), (D) and (F) as defined herein.

### BRIEF DESCRIPTION OF THE FIGURES

FIG. 1 shows the efficacy of the CPT1 inhibitors, Example 1 (Ex. 1, racemic mixture), tested in the fatty acid uptake assay using HEK293 cells with IC₅₀ of 0.3 μM,

FIG. 2 shows the efficacy of the CPT1 inhibitor by improved survival of Example 1-E1 (Ex. 1) (n=10) and Example 1-E2 (Exp. 1) (n=10) in SOD1 G93A mice compared to SOD1 G93A mice receiving either vehicle (n=9), Edaravone (n=10) and Riluzole (n=10).

### DETAILED DESCRIPTION OF THE INVENTION

### Definitions

As used herein, including the accompanying claims, the substituents and terms, which are collectively used, have the following meanings.

As used herein, the term “aryl” means a mono-, bi- or polycyclic aromatic system, for example unsubstituted or substituted phenyl, naphthyl, tetrahydronaphthyl, indenyl, indanyl, pentalenyl, fluorenyl and the like, preferably unsubstituted or substituted phenyl and naphthyl, particularly preferred unsubstituted or substituted phenyl.

As used herein, the term “heteroaryl” means an aromatic or partly unsaturated 5- or 6-membered ring which comprises one, two or three atoms selected from nitrogen, oxygen and/or sulphur, such as pyridyl, pyrazinyl, pyrimidinyl, pyridazinyl, 2-oxo-1,2-dihydropyridinyl, oxazolyl, oxydiazolyl, isoxazolyl, thiadiazolyl, tetrazolyl, pyrazolyl, imidazolyl, thiazolyl and thienyl. The term “heteroaryl” further refers to bicyclic aromatic or partly unsaturated groups comprising two 5- or 6-membered rings, in which one or both rings can contain one, two or three atoms selected from nitrogen, oxygen or sulphur, such as quinolinyl, isoquinolinyl, cinnolinyl, pyrazolo[1,5-a]pyridyl, imidazo[1,2-a]pyridyl, quinoxalinyl, benzothiazolyl, benzotriazolyl, indolyl, indazolyl. Preferred heteroaryl groups are pyridyl, pyrazolyl, thienyl and pyrazinyl.

As used herein, the term “4-, 5- or 6-membered saturated or partially unsaturated heterocyclyl” represents an unsubstituted or substituted saturated or partially unsaturated ring system containing 4, 5 or 6 ring atoms and containing in addition to C ring atoms one to three nitrogen atoms and/or an oxygen or sulfur atom or one or two oxygen and/or sulfur atoms. In a particular preferred embodiment the “4-, 5- or 6-membered saturated heterocyclyl” represents an unsubstituted or substituted saturated ring system containing 4, 5 or 6 ring atoms and containing in addition to C ring atoms one to three nitrogen atoms and/or an oxygen or sulfur atom or one or two oxygen and/or sulfur atoms. In a preferred embodiment, the 4-, 5- or 6-membered saturated heterocyclyl contains in addition to C ring atoms one N and optionally one additional heteroatom. The additional heteroatoms are preferably selected from O, N or S. Especially preferred are heterocycles with only one N as a heteroatom. Preferably, these substituted heterocycles are single or twofold substituted. The 4-, 5- or 6-membered saturated heterocycle may be substituted at the C atom(s), at the O atom(s), at the N atom(s) or at the S atom(s). Examples of 4-, 5- or 6-membered saturated heterocyclyl include, but are not limited to oxetanyl, azetidinyl, 1,3-diazetinyl, thietanyl, 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2,3-pyrrolin-2-yl, 2,3-pyrrolin-3-yl, 2,4-pyrrolin-2-yl, 2,4-pyrrolin-3-yl, 2,3-isoxazolin-3-yl, 3,4-isoxazolin-3-yl, 4,5-isoxazolin-3-yl, 2,3-isoxazolin-4-yl, 3,4-isoxazolin-4-yl, 4,5-isoxazolin-4-yl, 2,3-isoxazolin-5-yl, 3,4-isoxazolin-5-yl, 4,5-isoxazolin-5-yl, 2,3-isothiazolin-3-yl, 3,4-isothiazolin-3-yl, 4,5-isothiazolin-3-yl, 2,3-isothiazolin-4-yl, 3,4-isothiazolin-4-yl, 4,5-isothiazolin-4-yl, 2,3-isothiazolin-5-yl, 3,4-isothiazolin-5-yl, 4,5-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-piperidinyl, 3-piperidinyl, 4-piperidinyl, 1-piperazinyl, 2-piperazinyl, 1,3-dioxan-5-yl, 2-tetrahydropyranyl, 4-tetrahydropyranyl, 2-tetrahydrothienyl, 3-tetrahydropyridazinyl, 4-tetrahydropyridazinyl, 2-tetrahydropyrimidinyl, 4-tetrahydropyrimidinyl, 5-tetrahydropyrimidinyl, 2-tetrahydropyrazinyl, 1,3,5-tetrahydrotriazin-2-yl and 1,2,4-tetrahydrotriazin-3-yl, preferably piperidin-1-yl, 2-piperidinyl, 3-piperidinyl, 4-piperidinyl, 1-piperazinyl, 2-piperazinyl, 2-pyrrolidinyl, and 3-pyrrolidinyl, tetrahydropyridinyl, preferably 1,2,3,6-tetrahydropyridinyl, 1,2-oxazinyl, 1,3-oxazinyl, and 1,4-oxazinyl.

As used herein, the term “C₃-C₈-cycloalkyl” means a carbocyclic saturated ring system having 3 to 8 carbon atoms, preferably 3 to 6, particularly preferred 5 to 6 carbon atoms, for example cyclopropyl, cyclobutyl, cyclopentyl, cyclohexyl, cycloheptyl or cyclooctyl, preferably cyclopentyl and cyclohexyl.

The aryl, heteroaryl, 4-, 5- or 6-membered saturated or partially unsaturated heterocyclyl or the C₃-C₈-cycloalkyl, may be each optionally and independently substituted with one or more, preferably with one of the following residues:
—CN,
halogen, preferably —F or —Cl,
C₁-C₄-alkyl, preferably methyl,
halogen-C₁-C₄-alkyl, preferably difluoromethyl, or trifluoromethyl,
SO₂Me, or
CO₂C₁-C₄-alkyl, preferably CO₂Me.

As used herein, the term “C₁-C₄-alkyl” means a straight-chain or branched-chain alkyl group with 1 to 4 carbon atoms, respectively. Examples of straight-chain and branched groups include, but are not limited to, methyl, ethyl, propyl, isopropyl, butyl, isobutyl, and tert-butyl, preferably methyl and ethyl and most preferred methyl.

As used herein, the term “halogen-C₁-C₄-alkyl” means a straight-chain or branched alkyl group having 1 to 4 carbon atoms (as mentioned above), it being possible for the hydrogen atoms in these groups to be partly or completely replaced by halogen atoms as mentioned above, e.g. C₁-C₂-halogenalkyl such as chloromethyl, dichloromethyl, trichloromethyl, fluoromethyl, difluoromethyl, trifluoromethyl, chlorofluoromethyl, dichlorofluoromethyl, chlorodifluoromethyl, 1-fluoroethyl, 2-fluoroethyl, 2,2-difluoroethyl, 2,2,2-trifluoroethyl, 2-chloro-2fluoroethyl, 2-chloro-2,2-difluoroethyl, 2,2-dichloro-2-fluoroethyl, 2,2,2-trichloroethyl and pentafluoroethyl, preferably trifluoromethyl.

As used herein, the term “substitution” or “substituted” means one or more substituents commonly known in the art, or as specifically defined herein.

As used herein, the term “halogen” represents fluoro, chloro, bromo or iodo, preferably represents fluoro and chloro.

As used herein, the term “stereoisomer(s)” as it relates to a compound of formula (I) and to its intermediate compounds means any possible enantiomers or diastereomers of a compound of formula (I) and its salts or hydrates. In particular, the term “stereoisomer” means a single compound or a mixture of two or more compounds, wherein at least one chiral center is predominantly present in one definite isomeric form, in particular the S-enantiomer, the R-enantiomer and the racemate of a compound of formula (I). It is also possible that two or more stereogenic centers are predominantly present in one definite isomeric form of a derivative of a compound of formula (I) as defined above. In the sense of the present invention, “predominantly” has the meaning of at least 60%, preferably at least 70%, particularly preferably at least 80%, most preferably at least 90%. According to the present invention, also stereoisomers of a compound of formula (I) may be present as a salt or a hydrate.

As used herein, the term “salt(s)” as it relates to a compound of formula (I) as defined above means the physiologically acceptable acid addition salts and base salts of the compound of formula (I), i.e. its pharmaceutically or veterinarily acceptable salts, or its derivatives or its stereoisomers. Suitable acid addition salts are formed from acids which form non-toxic salts. Examples include but are not limited to the acetate, aspartate, benzoate, besylate, bicarbonate, carbonate, bisulphate, sulphate, borate, camsylate, citrate, edisylate, esylate, formate, fumarate, gluceptate, gluconate, glucuronate, hexafluorophosphate, hibenzate, hydrochloride/chloride, hydrobromide, bromide, hydroiodide, iodide, isethionate, lactate, malate, maleate, malonate, mesylate, methylsulphate, naphthylate, nicotinate, nitrate, orotate, oxalate, palmitate, pamoate, phosphate, hydrogen phosphate, dihydrogen phosphate, sacharate, stearate, succinate, tartrate, tosylate and trifluoroacetate salts. Suitable base salts are formed from bases which form non-toxic salts. Examples include but are not limited to the aluminium, arginine, benzathine, calcium, choline, diethylamine, diolamine, glycine, lysine, magnesium, meglumine, olamine, potassium, sodium, tromethamine and zinc salts.

As used herein, the term “hydrate(s)” as it relates to a compound of formula (I) means a compound of formula (I) or a stereoisomer or a salt thereof that includes water. “Hydrate(s)” are formed by the addition of water or its elements. In one embodiment, a compound of formula (I) as defined above or a stereoisomer or a salt thereof may form crystals that incorporate water into the crystalline structure without chemical alteration.

The terms stereoisomer, salt, and hydrate may also be used in conjunction with one another. For example, a stereoisomer of a compound of formula (I) may have a salt. Combinations of these terms are considered to be within the scope of the invention.

Technical terms are used by their common sense. If a specific meaning is conveyed to certain terms, definitions of terms will be given in the following in the context of which the terms are used.

The below mentioned general or preferred residue definitions apply both to the end products of the formula (I) and to specific embodiments thereof, and also, correspondingly, to the starting materials or to intermediates of formulae (A to (G) required in each case for the preparation. These residue definitions can be combined with one another at will, i.e. including combinations between the given preferred residues. Further, individual definitions may not apply.

As used herein, the terms “CPT-I inhibitor” or “inhibiting agent” mean any compound capable of down-regulating, decreasing, reducing, suppressing, or inactivating the amount and/or activity of the enzyme Carnitine-Palmitoyl-Transferase-1 (CPT-I), which is a key enzyme of the fatty acid oxidation pathway, and has the following catalytic activity: Palmitoyl-CoA+L-carnitine=CoA+L-palmitoylcarnitine. The enzyme is also known under the following synonyms: EC 2.3.1.21, CPT I, CPTI-L, Carnitine palmitoyltransferase IA, Carnitine palmitoyltransferase IB, Carnitine palmitoyltransferase 1C, CPT IM. Generally, CPT-I inhibitors or inhibiting agents may be proteins, oligo- and polypeptides, nucleic acids, genes, and chemical molecules. Suitable protein inhibitors may be, for example, monoclonal or polyclonal antibodies which bind to one of the enzymes described below. Inhibition of enzymes can be achieved by any of a variety of mechanisms known in the art, including, but not limited to, binding directly to the enzyme (e.g., enzyme inhibitor compound binding complex or substrate mimetic), denaturing or otherwise inactivating the enzyme, inhibiting the expression of a gene which encodes the enzyme (e.g., transcription to mRNA, translation to a nascent polypeptide) and/or final modifications to a mature protein.

As used herein, the term “inhibit” or “inhibiting” means any effect in down-regulating, decreasing, reducing, suppressing, or inactivating (also partially) the amount and/or activity of the Carnitine-Palmitoyl-Transferase-1 enzyme.

As used herein, the term “regulating the expression and/or activity” generally refers to any process that functions to control or modulate the quantity or activity (functionality) of a cellular component, particularly an enzyme. Static regulation maintains expression and/or activity at some given level. Up-regulation refers to a relative increase in expression and/or activity. Accordingly, down-regulation refers to a decrease in expression and/or activity. Down-regulation is synonymous with the inhibition of a given cellular component's expression and/or activity.

In general, CPT-I inhibitors can be identified by screening test compounds, for example a compound of formula (I) or a library of test compounds, for their ability to inhibit the Carnitine-Palmitoyl-Transferase-1 activity. In this context, cells or cell lysates may be tested for their ability to degrade palmitate by incubating the cells or cell lysates with radioactive palmitate and measuring the production of radioactive ketone bodies and/or the release of ¹⁴CO₂. Furthermore, it is possible to perform an in silico screen, based on the structure of a known enzyme involved in fatty acid oxidation.

### Novel Diazepane Derivative CPT-1 Inhibitors

As indicated above, there is a need for novel compounds, which specifically inhibit Carnitine-Palmitoyl-Transferase-1 (CPT-1) and that are suitable for use as a medicament. In particular, novel compounds are of interest that can be used in the amelioration, prevention and/or treatment of mental and neurological diseases, in particular of multiple sclerosis (MD), autoimmune encephalomyelitis, Parkinson's Disease (PD), and amyotrophic lateral sclerosis (ALS).

Therefore, a problem of the present invention was to provide novel compounds having the above-mentioned desired characteristics that are in particular suitable for use in the amelioration, prevention and/or treatment of mental and neurological diseases.

The present invention, in one aspect, relates to a compound of the formula (I)

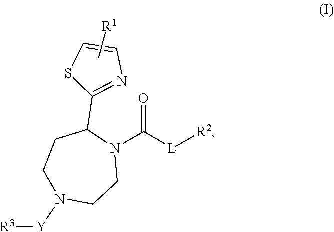

wherein
R¹ is unsubstituted or substituted aryl, preferably unsubstituted or substituted phenyl, naphthyl, tetrahydronaphthyl, indenyl, indanyl, pentalenyl, or fluorenyl, unsubstituted or substituted heteroaryl, preferably unsubstituted or substituted pyridyl, pyrazinyl, pyrimidinyl, pyridazinyl, 2-oxo-1,2-dihydropyridinyl, oxazolyl, oxydiazolyl, isoxazolyl, thiadiazolyl, tetrazolyl, pyrazolyl, imidazolyl, thiazolyl and thienyl, quinolinyl, isoquinolinyl, cinnolinyl, pyrazolo[1,5-a]pyridyl, imidazo[1,2-a]pyridyl, quinoxalinyl, benzothiazolyl, benzotriazolyl, indolyl, or indazolyl,
   unsubstituted or substituted 5- or 6-membered saturated or partially unsaturated heterocyclyl, or
   unsubstituted or substituted C₃-C₈-cycloalkyl, or cyclohexenyl;
L is a single bond, or a difunctional linker, preferably *—O—, *—OCH₂—, *—CH₂O—, *—CH₂—, *—CH₂—CH₂—, *—CH₂—CH₂—CH₂—, or *—CH₂—C(CH₃)₂—, or a trifunctional linker, preferably, *—CH═, wherein the * indicates the point of attachment to the carbonyl (C═O) group;
R² is unsubstituted or substituted phenyl, naphthyl, or pyridyl, or C₁-C₄-alkyl;
R³ is H, C₁-C₈-alkyl, halogen-C₁-C₄-alkyl, or C₃-C₈-cycloalkyl,
   unsubstituted or substituted 4, 5- or 6-membered saturated or partially unsaturated heterocyclyl, or
   unsubstituted or substituted phenyl;
Y is —(C═O)—, —(SO₂)— or a single bond;
or a stereoisomer, a pharmaceutically or veterinarily acceptable salt, hydrate or solvate thereof.

In one embodiment, the invention relates to a compound of the formula (I)

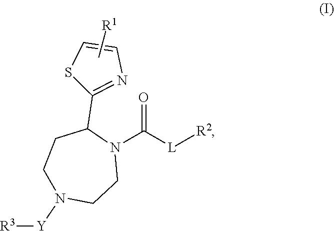

wherein
R¹ is unsubstituted or substituted aryl, preferably unsubstituted or substituted phenyl, naphthyl, tetrahydronaphthyl, indenyl, indanyl, pentalenyl, or fluorenyl,
   unsubstituted or substituted heteroaryl, preferably unsubstituted or substituted pyridyl, pyrazinyl, pyrimidinyl, pyridazinyl, 2-oxo-1,2-dihydropyridinyl, oxazolyl, oxydiazolyl, isoxazolyl, thiadiazolyl, tetrazolyl, pyrazolyl, imidazolyl, thiazolyl and thienyl, quinolinyl, isoquinolinyl, cinnolinyl, pyrazolo[1,5-a]pyridyl, imidazo[1,2-a]pyridyl, quinoxalinyl, benzothiazolyl, benzotriazolyl, indolyl, or indazolyl,
   unsubstituted or substituted 5- or 6-membered saturated or partially unsaturated heterocyclyl, or
   unsubstituted or substituted C₃-C₈-cycloalkyl, or cyclohexenyl;
L is a single bond, or a difunctional linker, preferably *—O—, *—OCH₂—, *—CH₂O—, *—CH₂—, *—CH₂—CH₂—, *—CH₂—CH₂—CH₂—, or *—CH₂—C(CH₃)₂—, wherein the * indicates the point of attachment to the carbonyl (C═O) group;
R² is unsubstituted or substituted phenyl, naphthyl, or pyridyl,
R³ is H, C₁-C₈-alkyl, halogen-C₁-C₄-alkyl, or C₃-C₈-cycloalkyl,
   unsubstituted or substituted 4, 5- or 6-membered saturated or partially unsaturated heterocyclyl, or
   unsubstituted or substituted phenyl;
Y is —(C═O)—, —(SO₂)— or a single bond;
or a stereoisomer, a pharmaceutically or veterinarily acceptable salt, hydrate or solvate thereof.

In a particularly preferred embodiment, Y is —(C═O)—.

The thiazole ring of the compounds of formula (I) may be substituted in position 4 or in position 5 with R¹.

In a particular preferred embodiment, the thiazole ring is substituted in position 4 with R¹.

Further included are pharmaceutically or veterinarily acceptable salts, hydrates or solvates of the compounds of formula (I) or its intermediate compounds disclosed herein.

As shown in the Examples, the inventors have now surprisingly and unexpectedly found that the compounds of formula (I) or a pharmaceutically or veterinarily acceptable salt, hydrate or solvate thereof, are useful in the amelioration, prevention and/or treatment of diseases caused by or related to delipidation of a neural tissue. Specifically it has been found that the compounds of formula (I) or a pharmaceutically or veterinarily acceptable salt, hydrate or solvate thereof inhibit the expression and/or activity of the enzyme Carnitine-Palmitoyl-Transferase-1 (CPT-1).

In the following, preferred groups of the compounds of formula (I) of the present invention are described. The preferred groups constitute preferred embodiments of the compounds of formula (I). Any combinations of the embodiments of the compounds of formula (I) of the invention described herein are considered to be within the scope of the invention.

In one embodiment, the invention relates to a compound of the formula (I) as defined herein, wherein
R¹ is unsubstituted or substituted aryl, preferably unsubstituted or substituted phenyl, naphthyl, tetrahydronaphthyl, indenyl, indanyl, pentalenyl, or fluorenyl,
   unsubstituted or substituted heteroaryl, preferably unsubstituted or substituted pyridyl, pyrazinyl, pyrimidinyl, pyridazinyl, 2-oxo-1,2-dihydropyridinyl, oxazolyl, oxydiazolyl, isoxazolyl, thiadiazolyl, tetrazolyl, pyrazolyl, imidazolyl, thiazolyl and thienyl, quinolinyl, isoquinolinyl, cinnolinyl, pyrazolo[1,5-a]pyridyl, imidazo[1,2-a]pyridyl, quinoxalinyl, benzothiazolyl, benzotriazolyl, indolyl, or indazolyl,
   unsubstituted or substituted 5- or 6-membered saturated or partially unsaturated heterocyclyl, or
   unsubstituted or substituted C₃-C₈-cycloalkyl, or cyclohexenyl;
   each R¹ being optionally and independently substituted with one to three of the following residues:
   —CN,
   halogen, preferably —F or —Cl,
   C₁-C₄-alkyl, preferably methyl,
   halogen-C₁-C₄-alkyl, preferably difluoromethyl, or trifluoromethyl,
   SO₂Me, or
   CO₂C₁-C₄-alkyl, preferably CO₂Me;
L is a single bond, or a difunctional linker, preferably *—O—, *—OCH₂—, *—CH₂O—, *—CH₂—, *—CH₂—CH₂—, *—CH₂—CH₂—CH₂—, or *—CH₂—C(CH₃)₂—, wherein the * indicates the point of attachment to the carbonyl (C═O) group;
R² is unsubstituted or substituted phenyl, naphthyl, or pyridyl;
   each R² being optionally and independently substituted with one to three, preferably with one of the following residues:
   —CN,
   halogen, preferably —F or —Cl,
   C₁-C₄-alkyl, preferably methyl,
   halogen-C₁-C₄-alkyl, preferably difluoromethyl, or trifluoromethyl,
   SO₂Me,
   CO₂C₁-C₄-alkyl, preferably CO₂Me,
   adamantyl,
   unsubstituted or substituted phenyl, being optionally substituted with one or more, preferably with one substituent selected from
   halogen, preferably —F or —Cl,
   halogen-C₁-C₄-alkyl, preferably trifluoromethyl,
   C₃-C₈-cycloalkyl, preferably cyclohexyl, or
   Pyridyl;
R³ is H, C₁-C₈-alkyl, halogen-C₁-C₄-alkyl, or C₃-C₈-cycloalkyl,
   unsubstituted or substituted 4, 5- or 6-membered saturated or partially unsaturated heterocyclyl, or
   unsubstituted or substituted phenyl;
   each 5- or 6-membered saturated or partially unsaturated heterocyclyl or phenyl being optionally and independently substituted with one or more, preferably with one of the following residues:
   —CN,
   halogen, preferably —F or —Cl;
   C₁-C₄-alkyl, preferably methyl;
   halogen-C₁-C₄-alkyl, preferably chloromethyl, dichloromethyl, trichloromethyl, fluoromethyl, difluoromethyl, trifluoromethyl, chlorofluoromethyl, dichlorofluoromethyl, chlorodifluoromethyl, 1-fluoroethyl, 2-fluoroethyl, 2,2-difluoroethyl, 2,2,2-trifluoroethyl, 2-chloro-2fluoroethyl, 2-chloro-2,2-difluoroethyl, 2,2-dichloro-2-fluoroethyl, 2,2,2-trichloroethyl, pentafluoroethyl, or 2,2-difluor-3-methyl-butyl, particularly preferred difluoromethyl, or trifluoromethyl,
   SO₂Me,
   CO₂C₁-C₄-alkyl, preferably CO₂Me, or
   CO—C₁-C₄-alky, preferably CO-Me,
and
Y is —(C═O)—, —(SO₂)— or a single bond;

In one embodiment, the invention relates to a compound of the formula (I) as defined herein, wherein
R¹ is unsubstituted or substituted phenyl, naphthyl, or tetrahydronaphthyl,
   unsubstituted or substituted pyridyl, pyrazinyl, pyrimidinyl, oxazolyl, oxydiazolyl, isoxazolyl, thiadiazolyl, tetrazolyl, pyrazolyl, imidazolyl, thiazolyl, thienyl, or quinolinyl,
   unsubstituted or substituted pyrrolidinyl, piperidinyl, tetrahydropiperidinyl, or piperazinyl, or
   unsubstituted or substituted cyclopentyl; cyclohexyl or cyclohexenyl;
L is a single bond, or a difunctional linker, preferably *—O—, *—OCH₂—, *—CH₂O—, *—CH₂—, or *—CH₂—CH₂—, wherein the * indicates the point of attachment to the carbonyl (C═O) group;
R² is unsubstituted or substituted phenyl, naphthyl, or pyridyl;
R³ is H, C₁-C₄-alkyl, halogen-C₁-C₄-alkyl, cyclopropyl, cyclobutyl, cyclopentyl or cyclohexyl,
   unsubstituted or substituted azetidinyl, pyrrolidinyl, piperidinyl, or oxetanyl,

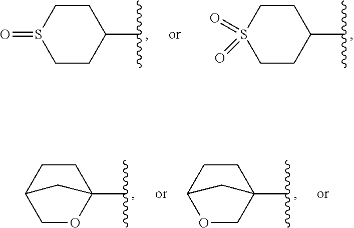 [SMILES: *C1CCS(=O)CC1 ; *C1CCS(=O)(=O)CC1 ; *C12CCC(CO1)C2 ; *C12CCC(C1)OC2]

or
unsubstituted or substituted phenyl; and
Y is —(C═O)—, —(SO₂)— or a single bond, preferably —(C═O)—.

In another embodiment, the invention relates to a compound of the formula (I) as defined herein, wherein
R¹ is unsubstituted or substituted phenyl, naphthyl, or tetrahydronaphthyl,
   unsubstituted or substituted pyridyl, pyrazinyl, pyrimidinyl, oxazolyl, oxydiazolyl, isoxazolyl, thiadiazolyl, tetrazolyl, pyrazolyl, imidazolyl, thiazolyl, thienyl, or quinolinyl,
   unsubstituted or substituted pyrrolidinyl, piperidinyl, tetrahydropiperidinyl, or piperazinyl, or
   unsubstituted or substituted cyclopentyl, cyclohexyl or cyclohexenyl,
   each R¹ being optionally and independently substituted with one or more, preferably with one of the following residues: —CN,
   halogen, preferably —F or —Cl,
   C₁-C₄-alkyl, preferably methyl,
   halogen-C₁-C₄-alkyl, preferably difluoromethyl, or trifluoromethyl,
   SO₂Me, or
   CO₂C₁-C₄-alkyl, preferably CO₂Me.

In another embodiment, the invention relates to a compound of the formula (I) as defined herein, wherein
R¹ is unsubstituted or substituted phenyl,
   unsubstituted or substituted pyridyl, pyrazolyl, thienyl, or quinolinyl,
   unsubstituted or substituted piperidinyl, or tetrahydropiperidinyl, or
   unsubstituted or substituted cyclohexyl or cyclohexenyl;
   each R¹ being optionally and independently substituted with one or more, preferably with one of the following residues:
   —CN,
   —F or —Cl,
   C₁-C₄-alkyl, preferably methyl,
   halogen-C₁-C₄-alkyl, preferably trifluoromethyl,
   SO₂Me, or
   CO₂C₁-C₄-alkyl, preferably CO₂Me.

In another embodiment, the invention relates to a compound of the formula (I) as defined herein, wherein
R¹ is unsubstituted or substituted phenyl or pyridyl,
   being optionally and independently substituted with one or more, preferably with one of the following residues:
   —F or —Cl,
   C₁-C₄-alkyl, preferably methyl,
   trifluoromethyl,
   SO₂Me, or
   CO₂C₁-C₄-alkyl, preferably CO₂Me.

In another embodiment, the invention relates to a compound of the formula (I) as defined herein, wherein
L is a single bond, *—CH₂O—, or *—CH₂—, preferably *—CH₂O—, wherein the * indicates the point of attachment to the carbonyl (C═O) group.

In another embodiment, the invention relates to a compound of the formula (I) as defined herein, wherein
R² is unsubstituted or substituted phenyl, naphthyl, or pyridyl; preferably phenyl,
   each R² being optionally and independently substituted with one or more, preferably with one of the following residues:
   —CN,
   halogen, preferably —F or —Cl,
   C₁-C₄-alkyl, preferably methyl,
   halogen-C₁-C₄-alkyl, preferably difluoromethyl, or trifluoromethyl,
   SO₂Me,
   CO₂C₁-C₄-alkyl, preferably CO₂Me,
   adamantyl,
   unsubstituted or substituted phenyl, being optionally substituted with one or more, preferably with one substituent selected from
   halogen, preferably —F or —Cl,
   halogen-C₁-C₄-alkyl, preferably trifluoromethyl,
   C₃-C₈-cycloalkyl, preferably cyclohexyl, or
   pyridyl.

In another embodiment, the invention relates to a compound of the formula (I) as defined herein, wherein
R³ is H, C₁-C₄-alkyl, halogen-C₁-C₄-alkyl, cyclopropyl, cyclobutyl, cyclopentyl or cyclohexyl,
   unsubstituted or substituted azetidinyl, pyrrolidinyl, piperidinyl, or oxetanyl,
   unsubstituted or substituted phenyl.
   each azetidinyl, pyrrolidinyl, piperidinyl, oxetanyl, or phenyl being optionally and independently substituted with one or more, preferably with one of the following residues:
   —CN,
   halogen, preferably —F or —Cl;
   C₁-C₄-alkyl, preferably methyl;
   halogen-C₁-C₄-alkyl, preferably chloromethyl, dichloromethyl, trichloromethyl, fluoromethyl, difluoromethyl, trifluoromethyl, chlorofluoromethyl, dichlorofluoromethyl, chlorodifluoromethyl, 1-fluoroethyl, 2-fluoroethyl, 2,2-difluoroethyl, 2,2,2-trifluoroethyl, 2-chloro-2fluoroethyl, 2-chloro-2,2-difluoroethyl, 2,2-dichloro-2-fluoroethyl, 2,2,2-trichloroethyl, pentafluoroethyl, or 2,2-difluor-3-methyl-butyl,
   particularly preferred difluoromethyl, or trifluoromethyl,
   SO₂Me,
   CO₂C₁-C₄-alkyl, preferably CO₂Me, or
   CO—C₁-C₄-alky, preferably CO-Me.

In another embodiment, the invention relates to a compound of the formula (I) as defined herein, wherein
R³ is C₁-C₄-alkyl, preferably methyl.

In one embodiment, the invention relates to a compound of the formula (I) as defined herein, wherein
R¹ is unsubstituted or substituted phenyl,
   unsubstituted or substituted pyridyl, pyrazolyl, thienyl, or quinolinyl,
   unsubstituted or substituted piperidinyl, tetrahydropiperidinyl, or piperazinyl, or
   unsubstituted or substituted cyclopentyl; cyclohexyl or cyclohexenyl;
   each R¹ being optionally and independently substituted with one of the following residues:
   —CN, —F or —Cl, C₁-C₄-alkyl, preferably methyl, or trifluoromethyl,
L is a single bond, *—OCH₂—, or *—CH₂—, or *, wherein the * indicates the point of attachment to the carbonyl (C═O) group;
R² is unsubstituted or substituted phenyl or naphthyl;
   each R² being optionally and independently substituted with one of the following residues:
   —CN, —F or —Cl, C₁-C₄-alkyl, preferably methyl, or trifluoromethyl,
   unsubstituted or substituted phenyl, being optionally substituted with one substituent selected from —F or —Cl, trifluoromethyl, or cyclohexyl, or pyridyl.
R³ is C₁-C₄-alkyl, halogen-C₁-C₄-alkyl, cyclopropyl, cyclobutyl, cyclopentyl or cyclohexyl,
   unsubstituted or substituted azetidinyl, pyrrolidinyl, piperidinyl, oxetanyl, or phenyl;
   each azetidinyl, pyrrolidinyl, piperidinyl, oxetanyl, or phenyl being optionally and independently substituted with one of the following residues:
   methyl; trifluoromethyl, 2,2-difluoro-3-methyl-butyl, or CO-Me and
Y is —(C═O)—.

In another embodiment, the invention relates to a compound of the formula (I), selected from

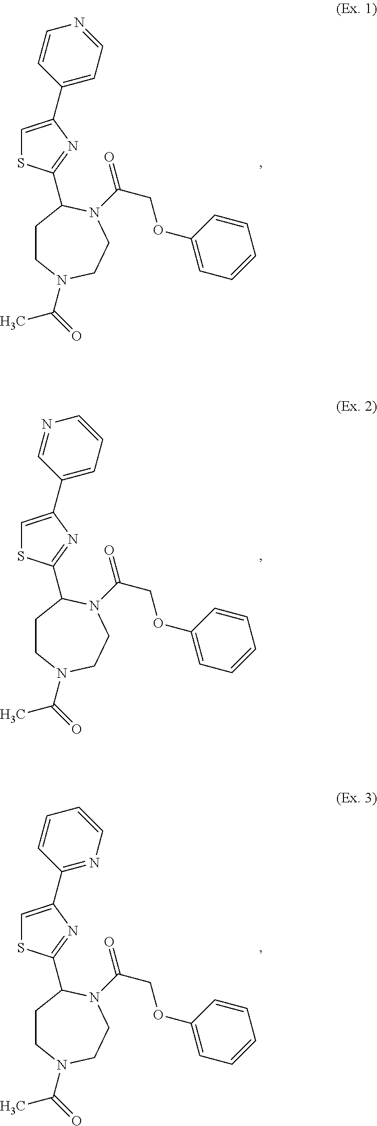 [SMILES: CC(=O)N1CCC(c2nc(-c3ccncc3)cs2)N(C(=O)COc2ccccc2)CC1 ; CC(=O)N1CCC(c2nc(-c3cccnc3)cs2)N(C(=O)COc2ccccc2)CC1 ; CC(=O)N1CCC(c2nc(-c3ccccn3)cs2)N(C(=O)COc2ccccc2)CC1]

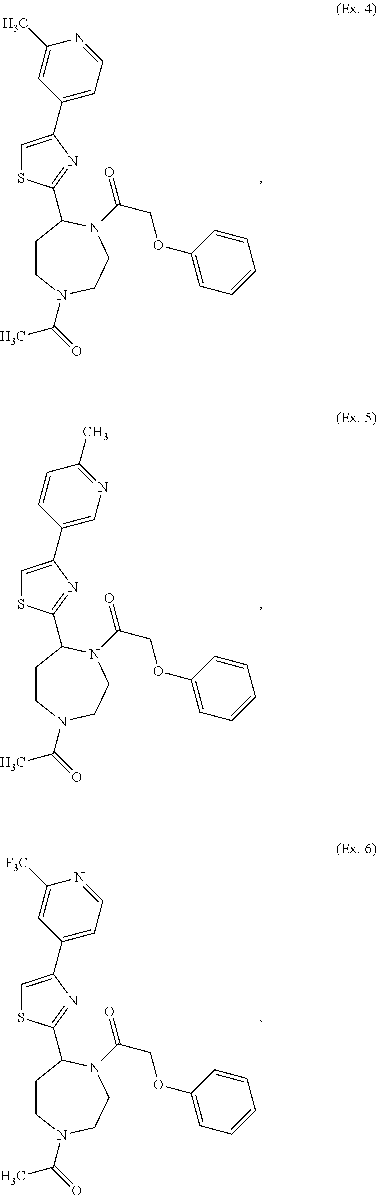 [SMILES: CC(=O)N1CCC(c2nc(-c3ccnc(C)c3)cs2)N(C(=O)COc2ccccc2)CC1 ; CC(=O)N1CCC(c2nc(-c3ccc(C)nc3)cs2)N(C(=O)COc2ccccc2)CC1 ; CC(=O)N1CCC(c2nc(-c3ccnc(C(F)(F)F)c3)cs2)N(C(=O)COc2ccccc2)CC1]

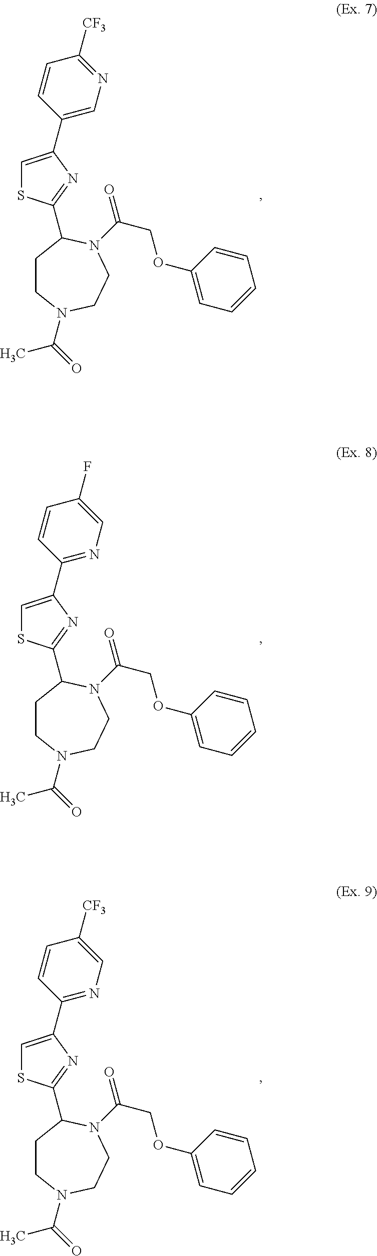 [SMILES: CC(=O)N1CCC(c2nc(-c3ccc(C(F)(F)F)nc3)cs2)N(C(=O)COc2ccccc2)CC1 ; CC(=O)N1CCC(c2nc(-c3ccc(F)cn3)cs2)N(C(=O)COc2ccccc2)CC1 ; CC(=O)N1CCC(c2nc(-c3ccc(C(F)(F)F)cn3)cs2)N(C(=O)COc2ccccc2)CC1]

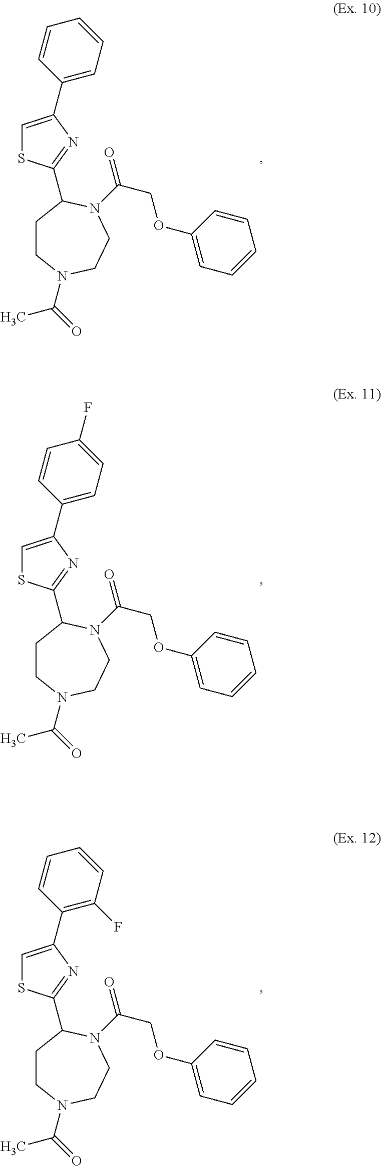 [SMILES: CC(=O)N1CCC(c2nc(-c3ccccc3)cs2)N(C(=O)COc2ccccc2)CC1 ; CC(=O)N1CCC(c2nc(-c3ccc(F)cc3)cs2)N(C(=O)COc2ccccc2)CC1 ; CC(=O)N1CCC(c2nc(-c3ccccc3F)cs2)N(C(=O)COc2ccccc2)CC1]

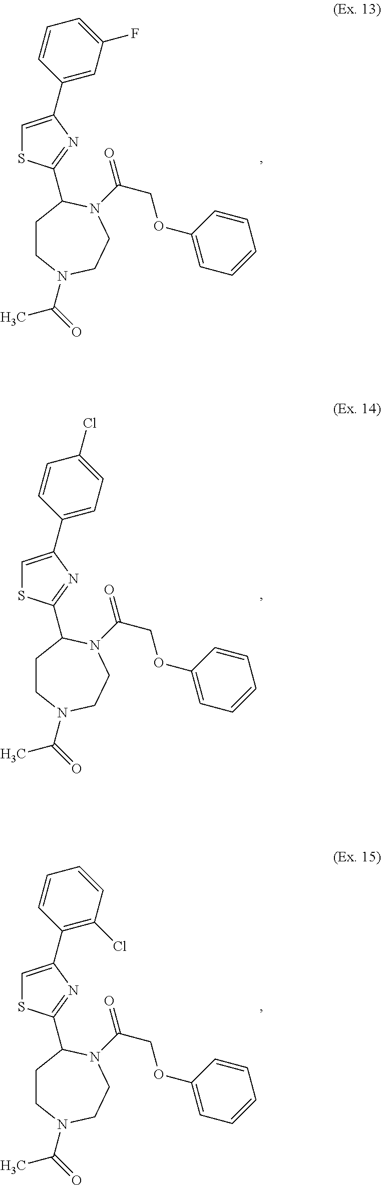 [SMILES: CC(=O)N1CCC(c2nc(-c3cccc(F)c3)cs2)N(C(=O)COc2ccccc2)CC1 ; CC(=O)N1CCC(c2nc(-c3ccc(Cl)cc3)cs2)N(C(=O)COc2ccccc2)CC1 ; CC(=O)N1CCC(c2nc(-c3ccccc3Cl)cs2)N(C(=O)COc2ccccc2)CC1]

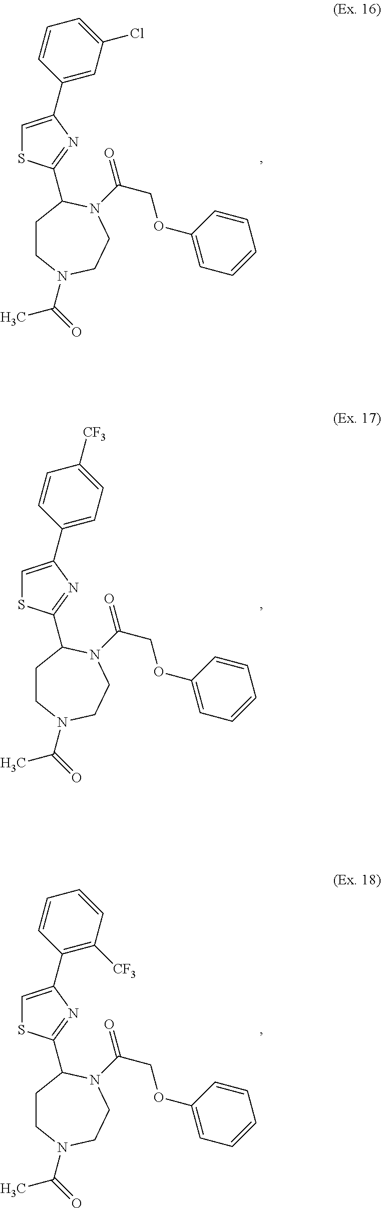 [SMILES: CC(=O)N1CCC(c2nc(-c3cccc(Cl)c3)cs2)N(C(=O)COc2ccccc2)CC1 ; CC(=O)N1CCC(c2nc(-c3ccc(C(F)(F)F)cc3)cs2)N(C(=O)COc2ccccc2)CC1 ; CC(=O)N1CCC(c2nc(-c3ccccc3C(F)(F)F)cs2)N(C(=O)COc2ccccc2)CC1]

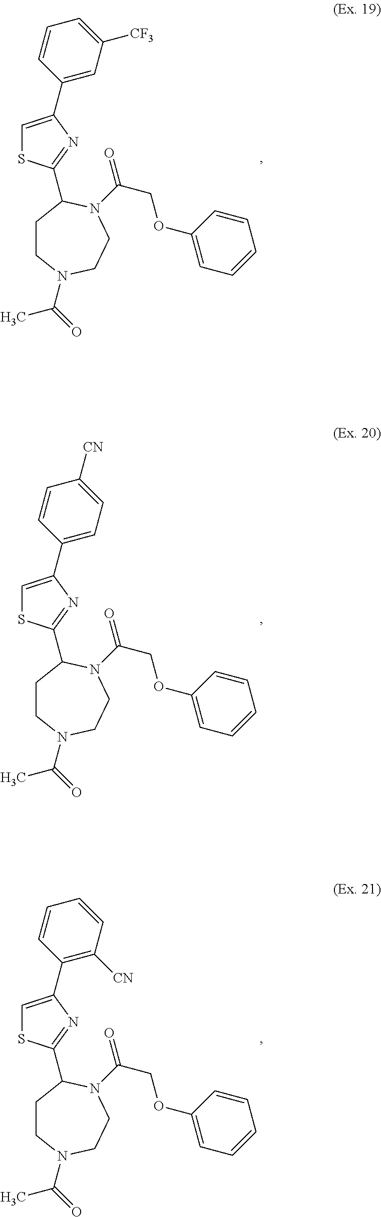 [SMILES: CC(=O)N1CCC(c2nc(-c3cccc(C(F)(F)F)c3)cs2)N(C(=O)COc2ccccc2)CC1 ; CC(=O)N1CCC(c2nc(-c3ccc(C#N)cc3)cs2)N(C(=O)COc2ccccc2)CC1 ; CC(=O)N1CCC(c2nc(-c3ccccc3C#N)cs2)N(C(=O)COc2ccccc2)CC1]

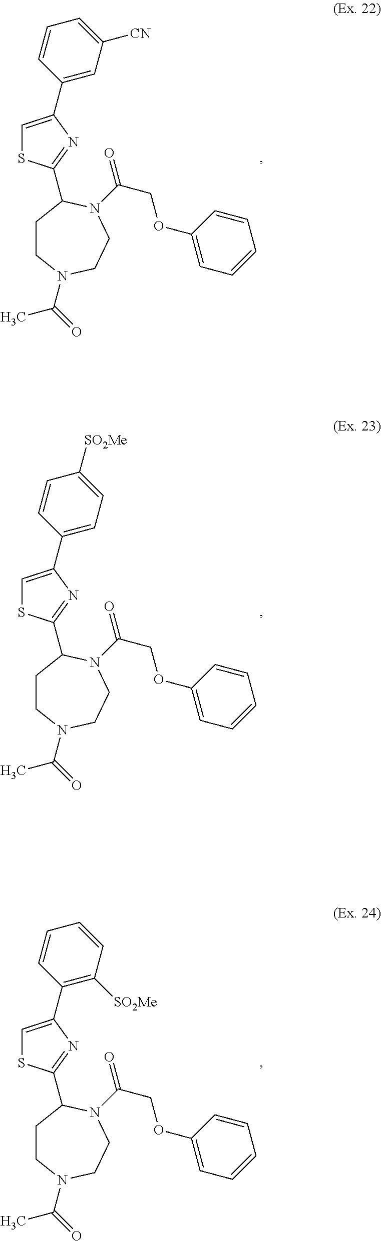 [SMILES: CC(=O)N1CCC(c2nc(-c3cccc(C#N)c3)cs2)N(C(=O)COc2ccccc2)CC1 ; CC(=O)N1CCC(c2nc(-c3ccc(S(C)(=O)=O)cc3)cs2)N(C(=O)COc2ccccc2)CC1 ; CC(=O)N1CCC(c2nc(-c3ccccc3S(C)(=O)=O)cs2)N(C(=O)COc2ccccc2)CC1]

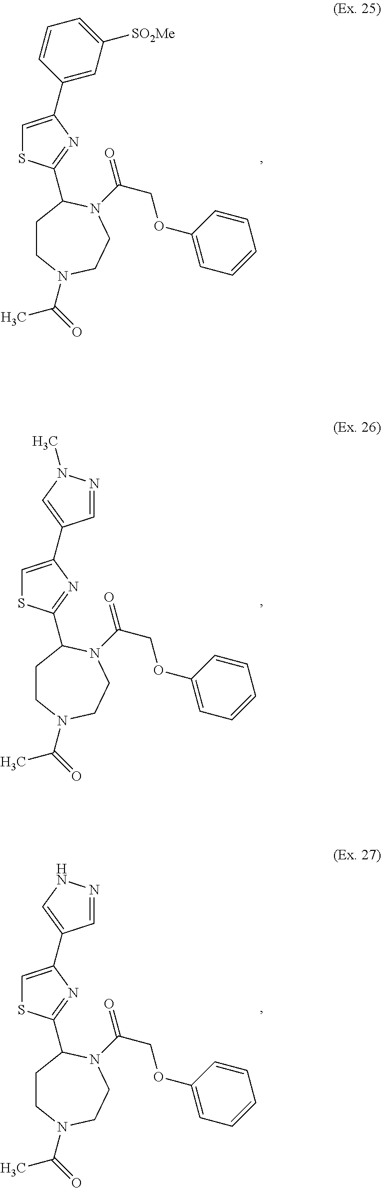 [SMILES: CC(=O)N1CCC(c2nc(-c3cccc(S(C)(=O)=O)c3)cs2)N(C(=O)COc2ccccc2)CC1 ; CC(=O)N1CCC(c2nc(-c3cnn(C)c3)cs2)N(C(=O)COc2ccccc2)CC1 ; CC(=O)N1CCC(c2nc(-c3cn[nH]c3)cs2)N(C(=O)COc2ccccc2)CC1]

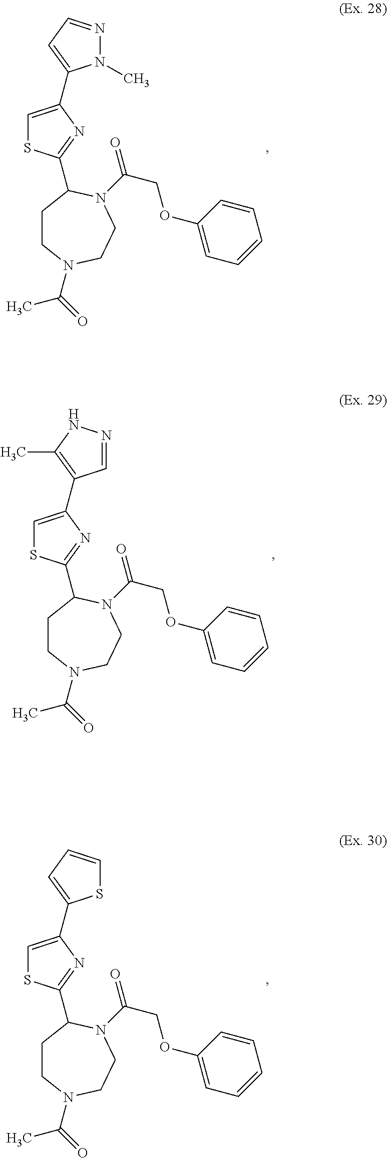 [SMILES: CC(=O)N1CCC(c2nc(-c3ccnn3C)cs2)N(C(=O)COc2ccccc2)CC1 ; CC(=O)N1CCC(c2nc(-c3cn[nH]c3C)cs2)N(C(=O)COc2ccccc2)CC1 ; CC(=O)N1CCC(c2nc(-c3cccs3)cs2)N(C(=O)COc2ccccc2)CC1]

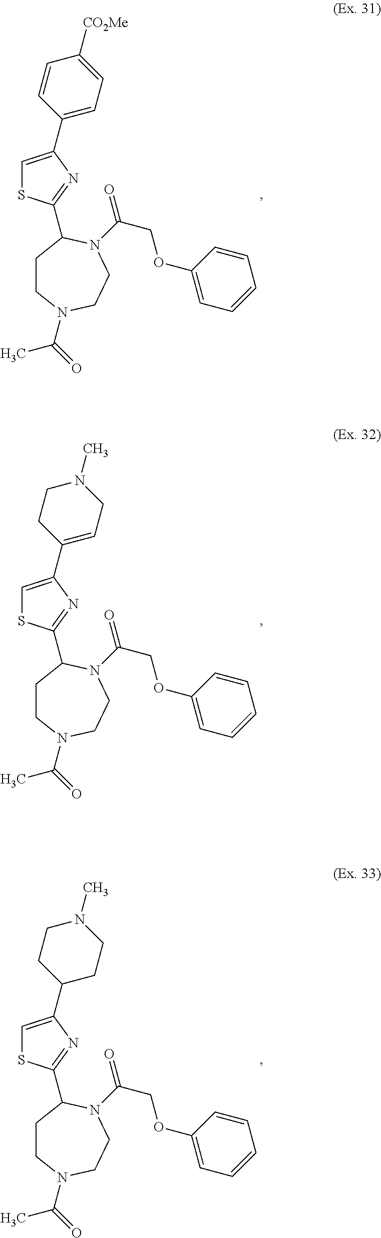 [SMILES: COC(=O)c1ccc(-c2csc(C3CCN(C(C)=O)CCN3C(=O)COc3ccccc3)n2)cc1 ; CC(=O)N1CCC(c2nc(C3=CCN(C)CC3)cs2)N(C(=O)COc2ccccc2)CC1 ; CC(=O)N1CCC(c2nc(C3CCN(C)CC3)cs2)N(C(=O)COc2ccccc2)CC1]

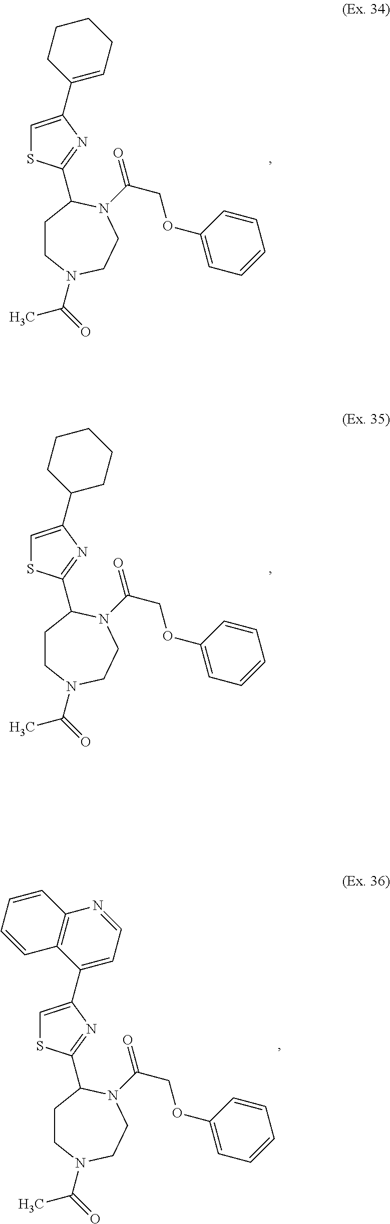 [SMILES: CC(=O)N1CCC(c2nc(C3=CCCCC3)cs2)N(C(=O)COc2ccccc2)CC1 ; CC(=O)N1CCC(c2nc(C3CCCCC3)cs2)N(C(=O)COc2ccccc2)CC1 ; CC(=O)N1CCC(c2nc(-c3ccnc4ccccc34)cs2)N(C(=O)COc2ccccc2)CC1]

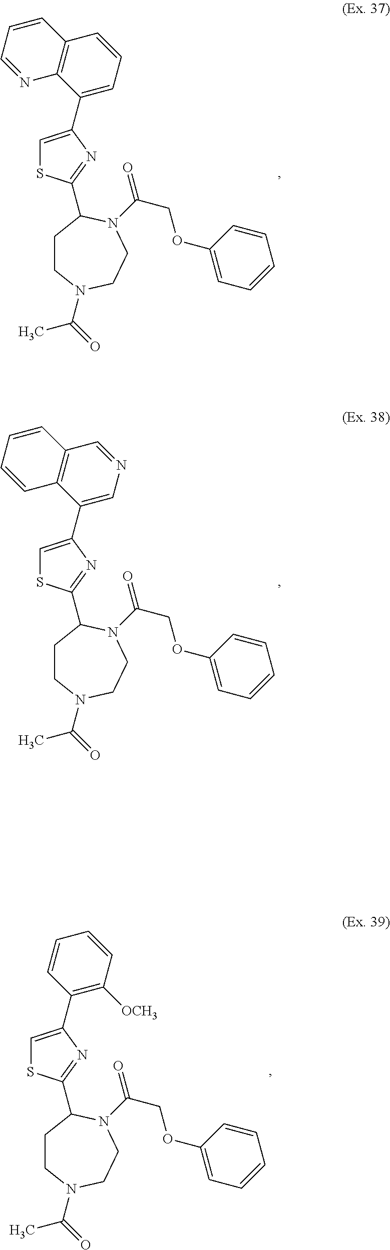 [SMILES: CC(=O)N1CCC(c2nc(-c3cccc4cccnc34)cs2)N(C(=O)COc2ccccc2)CC1 ; CC(=O)N1CCC(c2nc(-c3cncc4ccccc34)cs2)N(C(=O)COc2ccccc2)CC1 ; COc1ccccc1-c1csc(C2CCN(C(C)=O)CCN2C(=O)COc2ccccc2)n1]

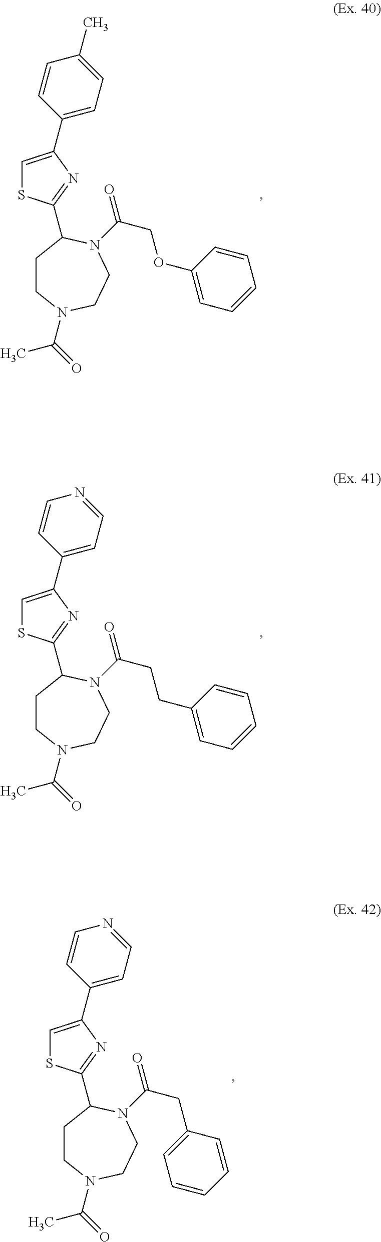 [SMILES: CC(=O)N1CCC(c2nc(-c3ccc(C)cc3)cs2)N(C(=O)COc2ccccc2)CC1 ; CC(=O)N1CCC(c2nc(-c3ccncc3)cs2)N(C(=O)CCc2ccccc2)CC1 ; CC(=O)N1CCC(c2nc(-c3ccncc3)cs2)N(C(=O)Cc2ccccc2)CC1]

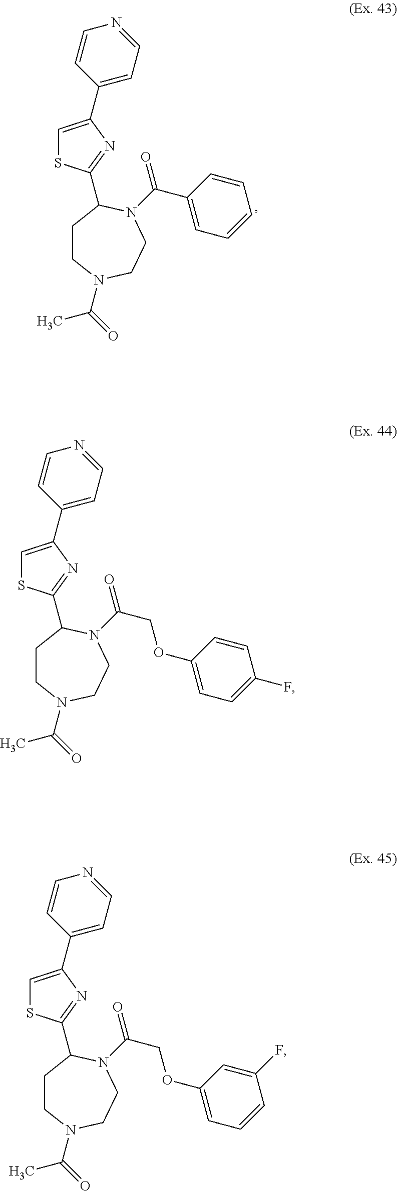 [SMILES: CC(=O)N1CCC(c2nc(-c3ccncc3)cs2)N(C(=O)c2ccccc2)CC1 ; CC(=O)N1CCC(c2nc(-c3ccncc3)cs2)N(C(=O)COc2ccc(F)cc2)CC1 ; CC(=O)N1CCC(c2nc(-c3ccncc3)cs2)N(C(=O)COc2cccc(F)c2)CC1]

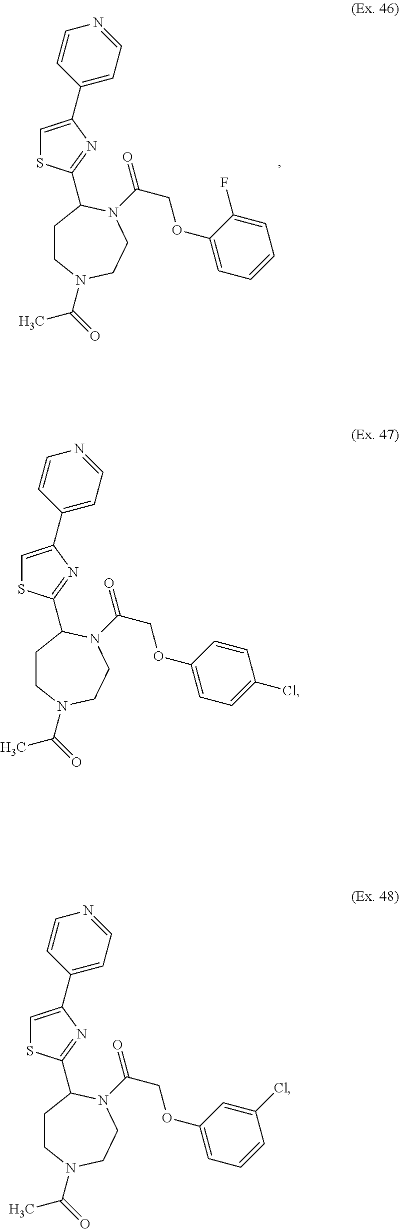 [SMILES: CC(=O)N1CCC(c2nc(-c3ccncc3)cs2)N(C(=O)COc2ccccc2F)CC1 ; CC(=O)N1CCC(c2nc(-c3ccncc3)cs2)N(C(=O)COc2ccc(Cl)cc2)CC1 ; CC(=O)N1CCC(c2nc(-c3ccncc3)cs2)N(C(=O)COc2cccc(Cl)c2)CC1]

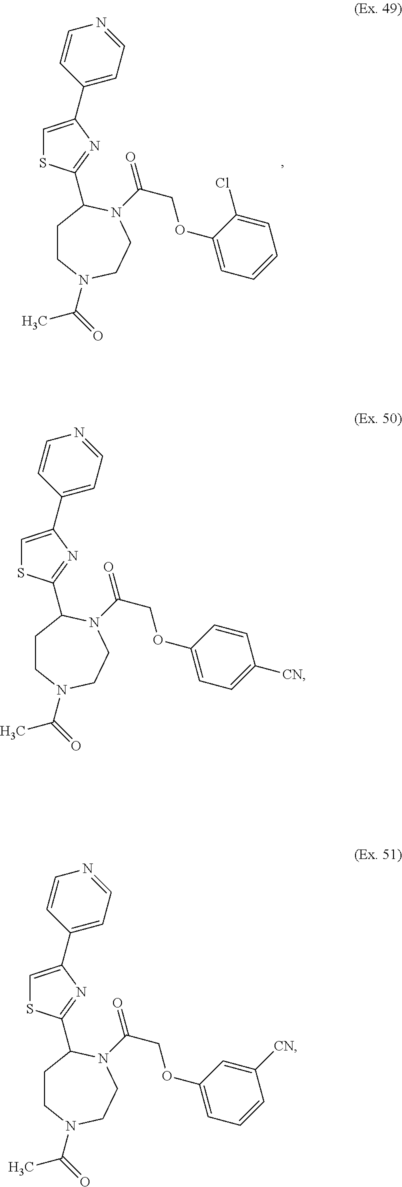 [SMILES: CC(=O)N1CCC(c2nc(-c3ccncc3)cs2)N(C(=O)COc2ccccc2Cl)CC1 ; CC(=O)N1CCC(c2nc(-c3ccncc3)cs2)N(C(=O)COc2ccc(C#N)cc2)CC1 ; CC(=O)N1CCC(c2nc(-c3ccncc3)cs2)N(C(=O)COc2cccc(C#N)c2)CC1]

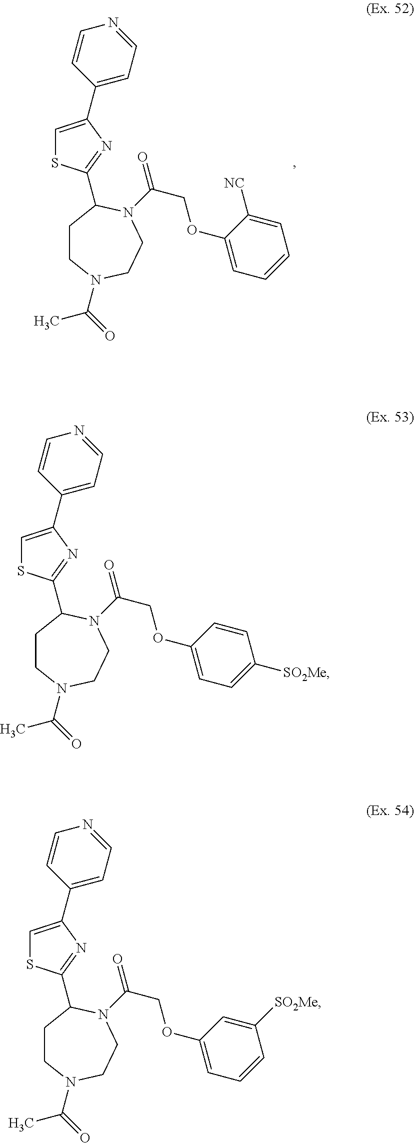 [SMILES: CC(=O)N1CCC(c2nc(-c3ccncc3)cs2)N(C(=O)COc2ccccc2C#N)CC1 ; CC(=O)N1CCC(c2nc(-c3ccncc3)cs2)N(C(=O)COc2ccc(S(C)(=O)=O)cc2)CC1 ; CC(=O)N1CCC(c2nc(-c3ccncc3)cs2)N(C(=O)COc2cccc(S(C)(=O)=O)c2)CC1]

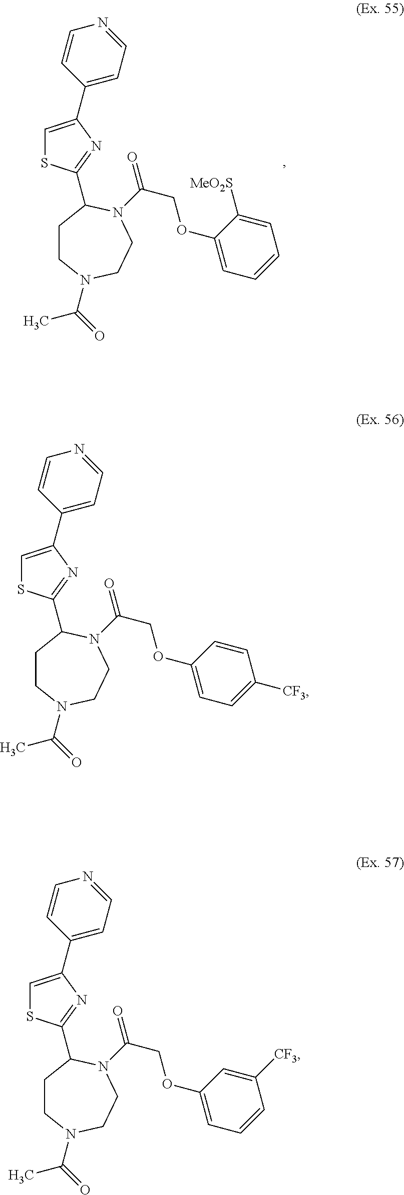 [SMILES: CC(=O)N1CCC(c2nc(-c3ccncc3)cs2)N(C(=O)COc2ccccc2S(C)(=O)=O)CC1 ; CC(=O)N1CCC(c2nc(-c3ccncc3)cs2)N(C(=O)COc2ccc(C(F)(F)F)cc2)CC1 ; CC(=O)N1CCC(c2nc(-c3ccncc3)cs2)N(C(=O)COc2cccc(C(F)(F)F)c2)CC1]

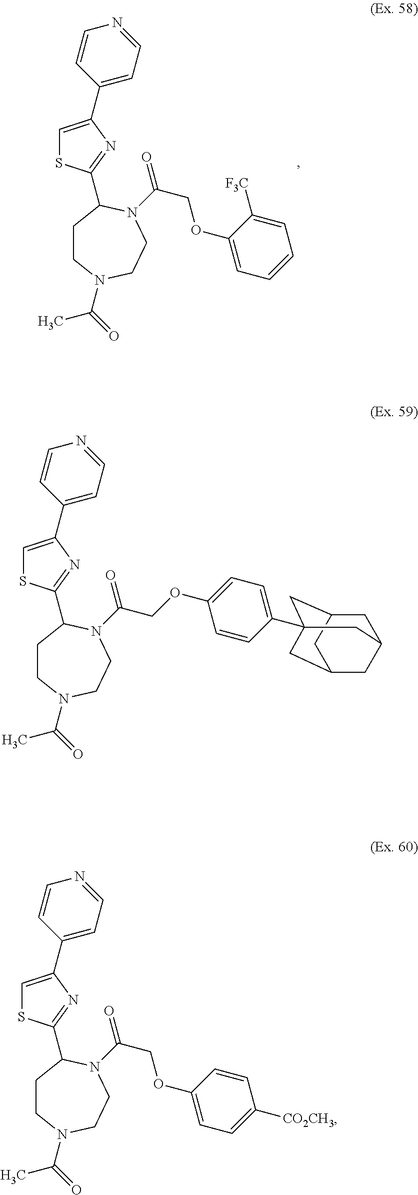 [SMILES: CC(=O)N1CCC(c2nc(-c3ccncc3)cs2)N(C(=O)COc2ccccc2C(F)(F)F)CC1 ; CC(=O)N1CCC(c2nc(-c3ccncc3)cs2)N(C(=O)COc2ccc(C34CC5CC(CC(C5)C3)C4)cc2)CC1 ; COC(=O)c1ccc(OCC(=O)N2CCN(C(C)=O)CCC2c2nc(-c3ccncc3)cs2)cc1]

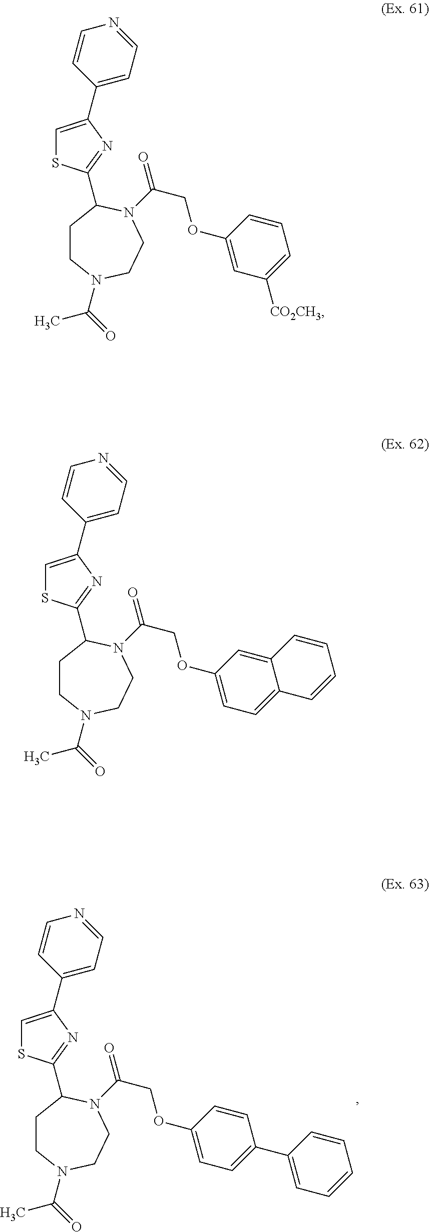 [SMILES: COC(=O)c1cccc(OCC(=O)N2CCN(C(C)=O)CCC2c2nc(-c3ccncc3)cs2)c1 ; CC(=O)N1CCC(c2nc(-c3ccncc3)cs2)N(C(=O)COc2ccc3ccccc3c2)CC1 ; CC(=O)N1CCC(c2nc(-c3ccncc3)cs2)N(C(=O)COc2ccc(-c3ccccc3)cc2)CC1]

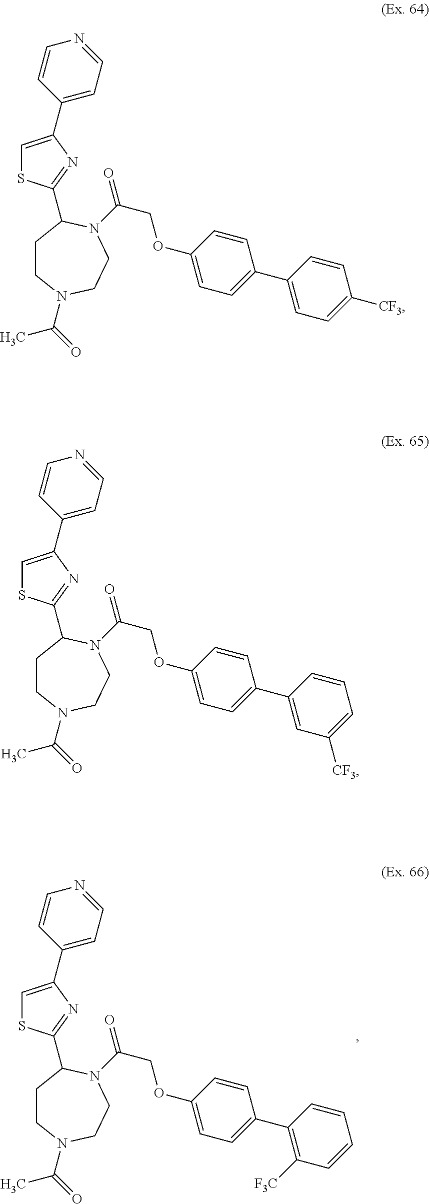 [SMILES: CC(=O)N1CCC(c2nc(-c3ccncc3)cs2)N(C(=O)COc2ccc(-c3ccc(C(F)(F)F)cc3)cc2)CC1 ; CC(=O)N1CCC(c2nc(-c3ccncc3)cs2)N(C(=O)COc2ccc(-c3cccc(C(F)(F)F)c3)cc2)CC1 ; CC(=O)N1CCC(c2nc(-c3ccncc3)cs2)N(C(=O)COc2ccc(-c3ccccc3C(F)(F)F)cc2)CC1]

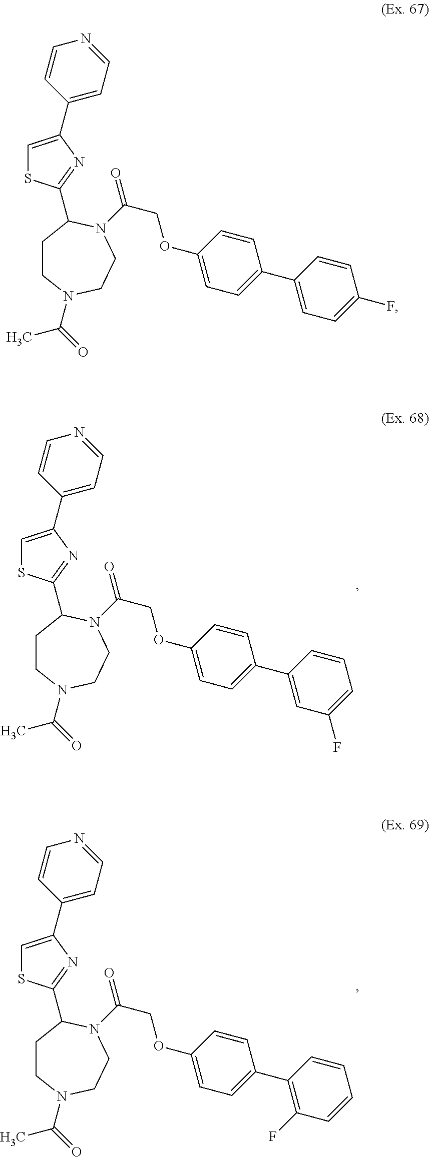 [SMILES: CC(=O)N1CCC(c2nc(-c3ccncc3)cs2)N(C(=O)COc2ccc(-c3ccc(F)cc3)cc2)CC1 ; CC(=O)N1CCC(c2nc(-c3ccncc3)cs2)N(C(=O)COc2ccc(-c3cccc(F)c3)cc2)CC1 ; CC(=O)N1CCC(c2nc(-c3ccncc3)cs2)N(C(=O)COc2ccc(-c3ccccc3F)cc2)CC1]

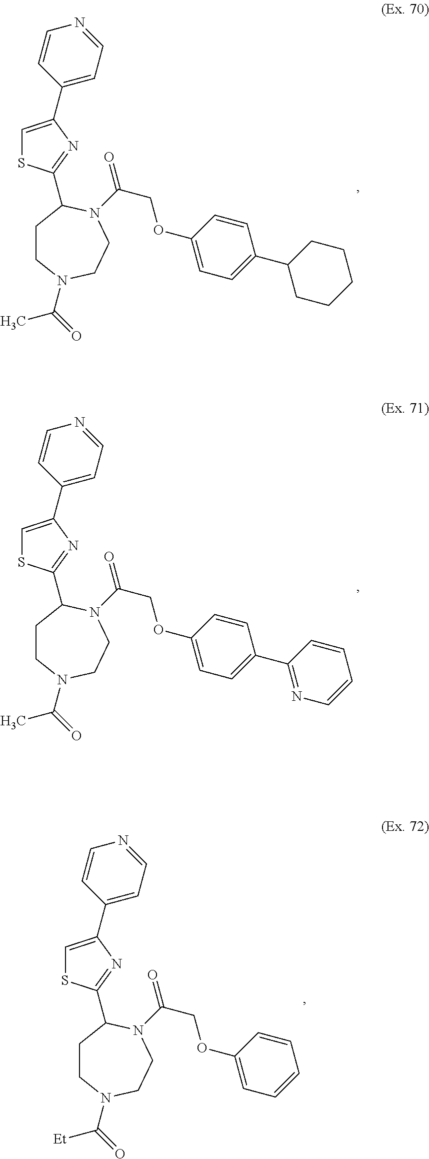 [SMILES: CC(=O)N1CCC(c2nc(-c3ccncc3)cs2)N(C(=O)COc2ccc(C3CCCCC3)cc2)CC1 ; CC(=O)N1CCC(c2nc(-c3ccncc3)cs2)N(C(=O)COc2ccc(-c3ccccn3)cc2)CC1 ; CCC(=O)N1CCC(c2nc(-c3ccncc3)cs2)N(C(=O)COc2ccccc2)CC1]

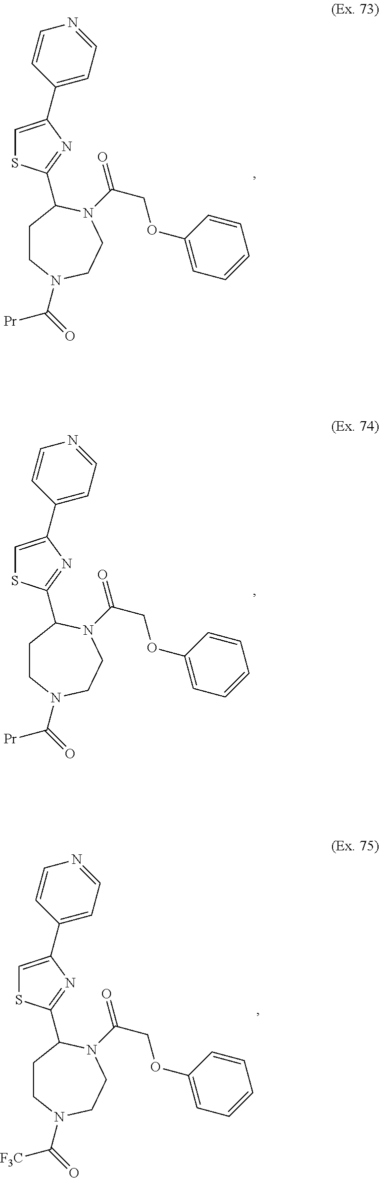

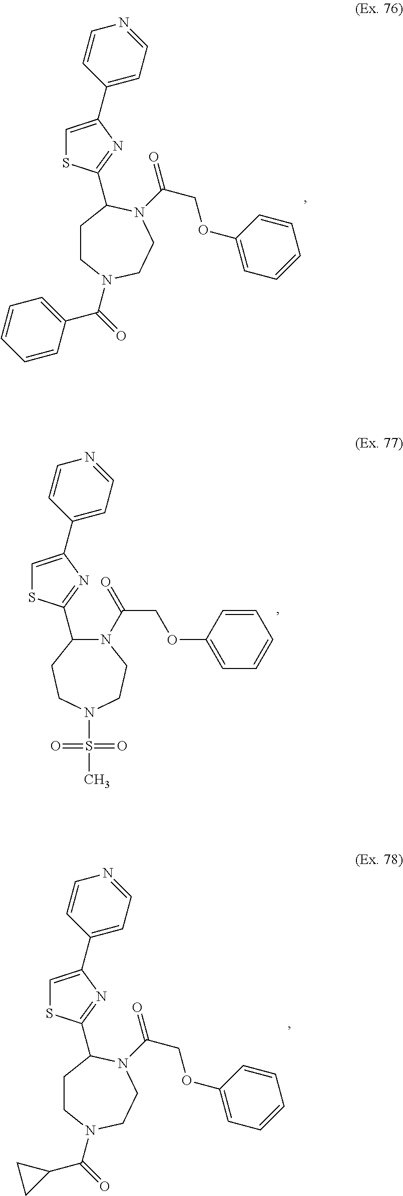 [SMILES: O=C(c1ccccc1)N1CCC(c2nc(-c3ccncc3)cs2)N(C(=O)COc2ccccc2)CC1 ; CS(=O)(=O)N1CCC(c2nc(-c3ccncc3)cs2)N(C(=O)COc2ccccc2)CC1 ; O=C(C1CC1)N1CCC(c2nc(-c3ccncc3)cs2)N(C(=O)COc2ccccc2)CC1]

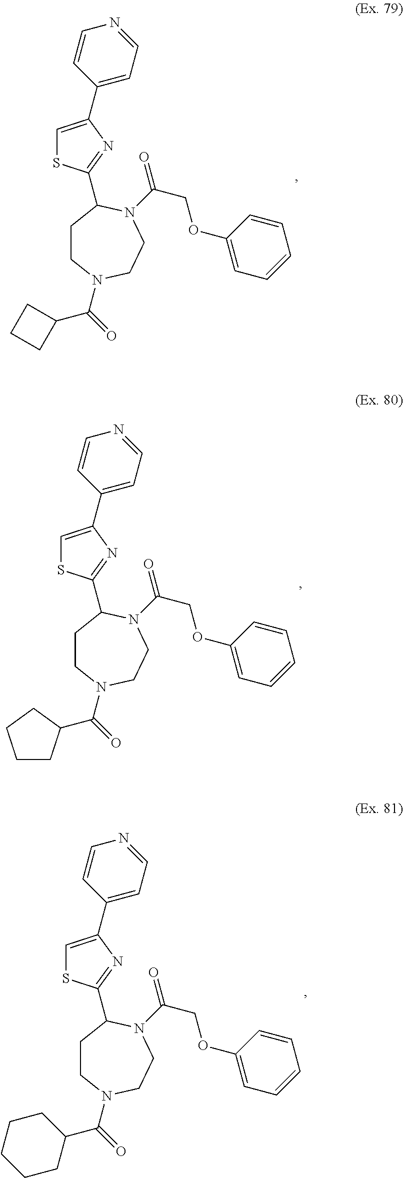 [SMILES: O=C(C1CCC1)N1CCC(c2nc(-c3ccncc3)cs2)N(C(=O)COc2ccccc2)CC1 ; O=C(C1CCCC1)N1CCC(c2nc(-c3ccncc3)cs2)N(C(=O)COc2ccccc2)CC1 ; O=C(C1CCCCC1)N1CCC(c2nc(-c3ccncc3)cs2)N(C(=O)COc2ccccc2)CC1]

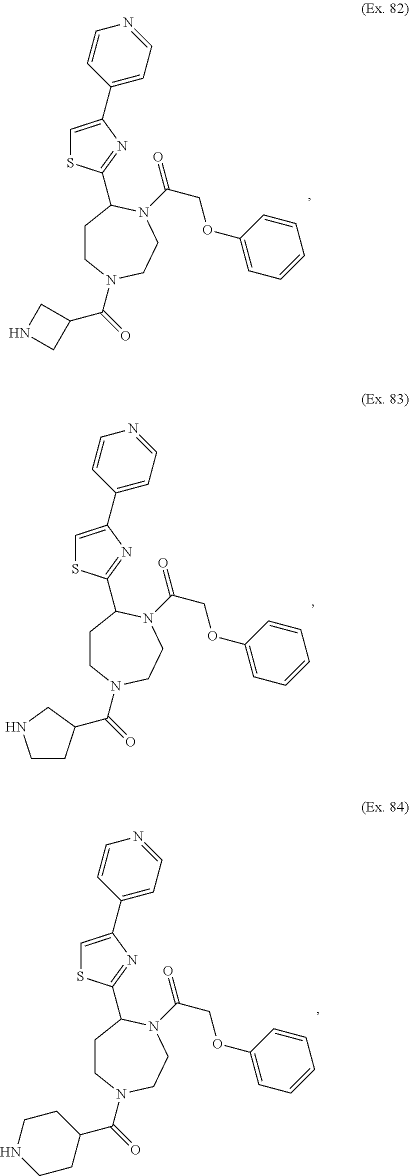 [SMILES: O=C(C1CNC1)N1CCC(c2nc(-c3ccncc3)cs2)N(C(=O)COc2ccccc2)CC1 ; O=C(C1CCNC1)N1CCC(c2nc(-c3ccncc3)cs2)N(C(=O)COc2ccccc2)CC1 ; O=C(C1CCNCC1)N1CCC(c2nc(-c3ccncc3)cs2)N(C(=O)COc2ccccc2)CC1]

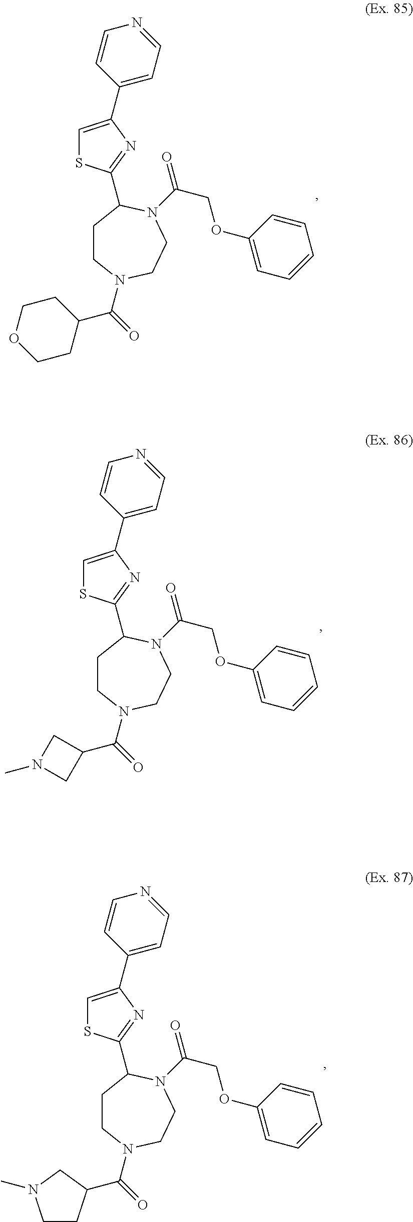 [SMILES: O=C(C1CCOCC1)N1CCC(c2nc(-c3ccncc3)cs2)N(C(=O)COc2ccccc2)CC1 ; CN1CC(C(=O)N2CCC(c3nc(-c4ccncc4)cs3)N(C(=O)COc3ccccc3)CC2)C1 ; CN1CCC(C(=O)N2CCC(c3nc(-c4ccncc4)cs3)N(C(=O)COc3ccccc3)CC2)C1]

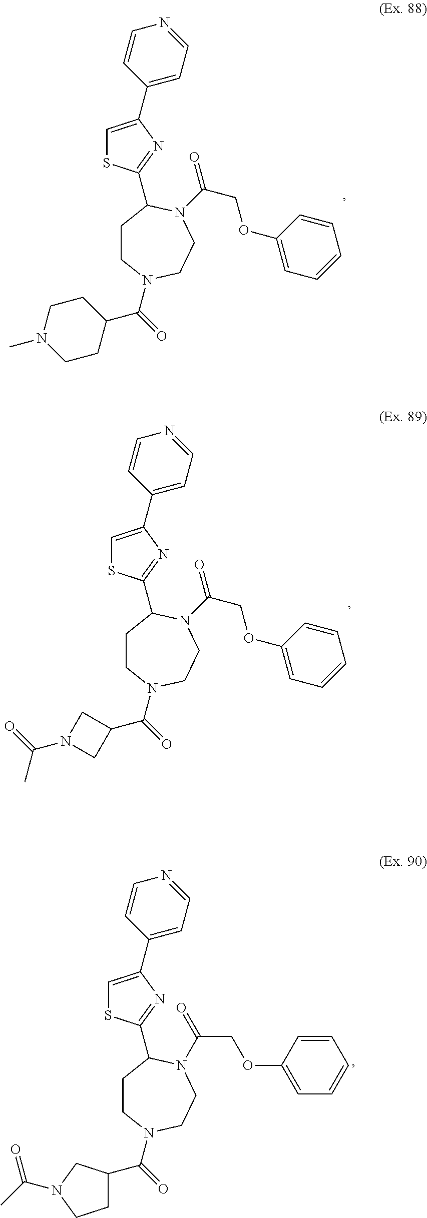 [SMILES: CN1CCC(C(=O)N2CCC(c3nc(-c4ccncc4)cs3)N(C(=O)COc3ccccc3)CC2)CC1 ; CC(=O)N1CC(C(=O)N2CCC(c3nc(-c4ccncc4)cs3)N(C(=O)COc3ccccc3)CC2)C1 ; CC(=O)N1CCC(C(=O)N2CCC(c3nc(-c4ccncc4)cs3)N(C(=O)COc3ccccc3)CC2)C1]

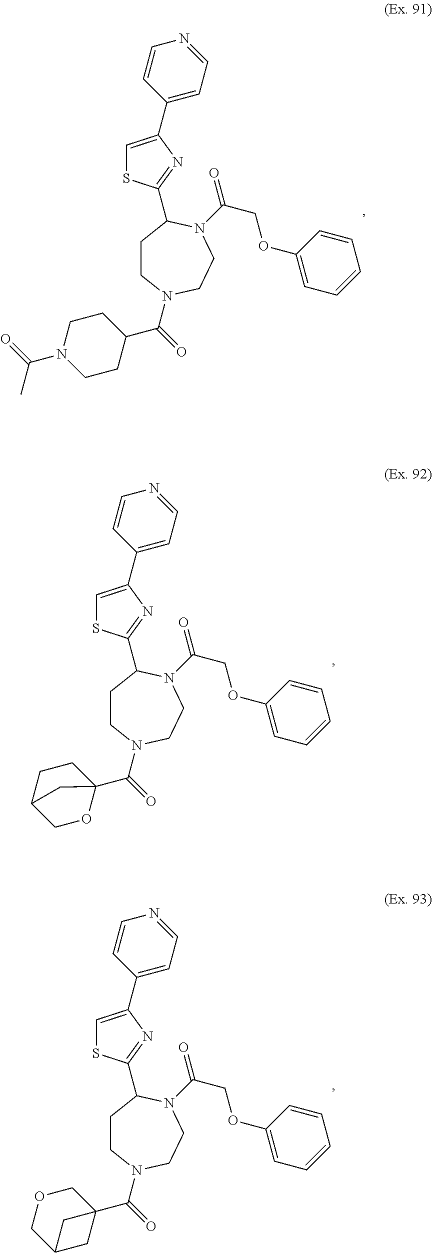 [SMILES: CC(=O)N1CCC(C(=O)N2CCC(c3nc(-c4ccncc4)cs3)N(C(=O)COc3ccccc3)CC2)CC1 ; O=C(COc1ccccc1)N1CCN(C(=O)C23CCC(CO2)C3)CCC1c1nc(-c2ccncc2)cs1 ; O=C(COc1ccccc1)N1CCN(C(=O)C23COCC(C2)C3)CCC1c1nc(-c2ccncc2)cs1]

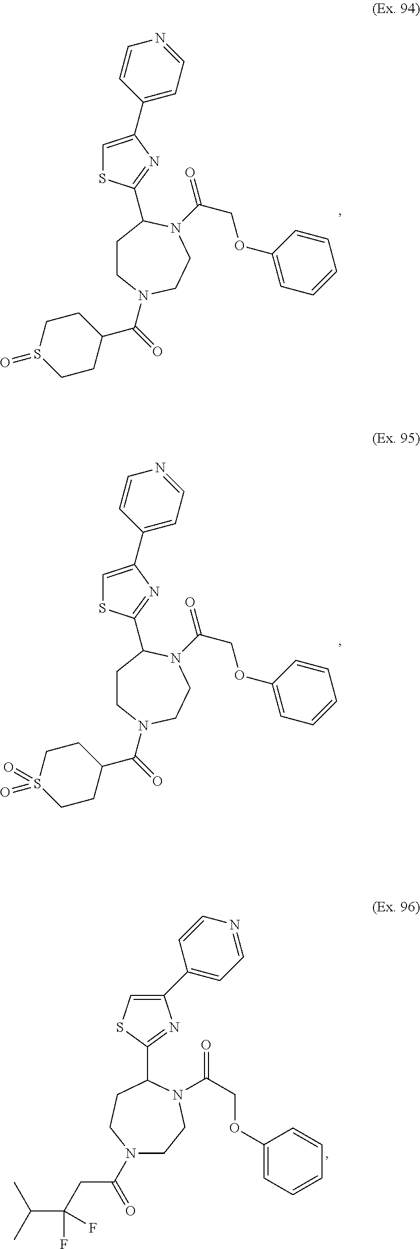 [SMILES: O=C(C1CCS(=O)CC1)N1CCC(c2nc(-c3ccncc3)cs2)N(C(=O)COc2ccccc2)CC1 ; O=C(C1CCS(=O)(=O)CC1)N1CCC(c2nc(-c3ccncc3)cs2)N(C(=O)COc2ccccc2)CC1 ; CC(C)C(F)(F)CC(=O)N1CCC(c2nc(-c3ccncc3)cs2)N(C(=O)COc2ccccc2)CC1]

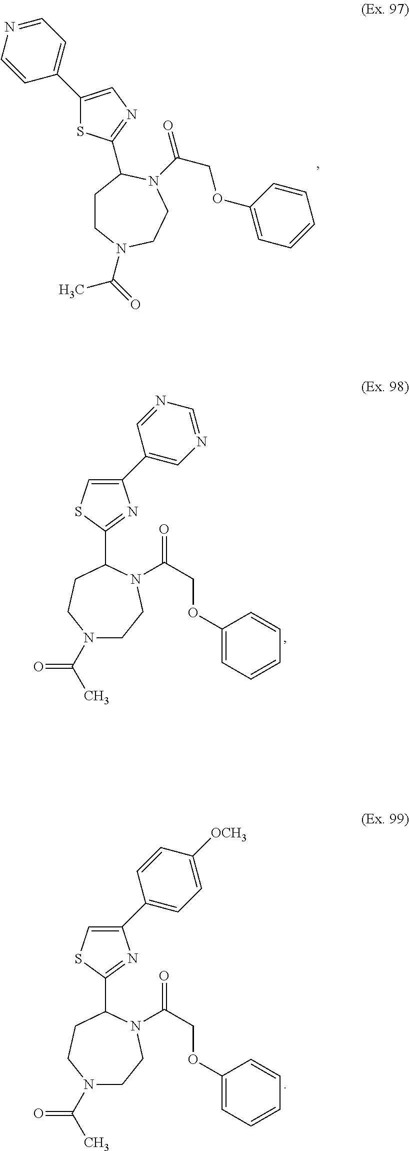 [SMILES: CC(=O)N1CCC(c2ncc(-c3ccncc3)s2)N(C(=O)COc2ccccc2)CC1 ; CC(=O)N1CCC(c2nc(-c3cncnc3)cs2)N(C(=O)COc2ccccc2)CC1 ; COc1ccc(-c2csc(C3CCN(C(C)=O)CCN3C(=O)COc3ccccc3)n2)cc1]

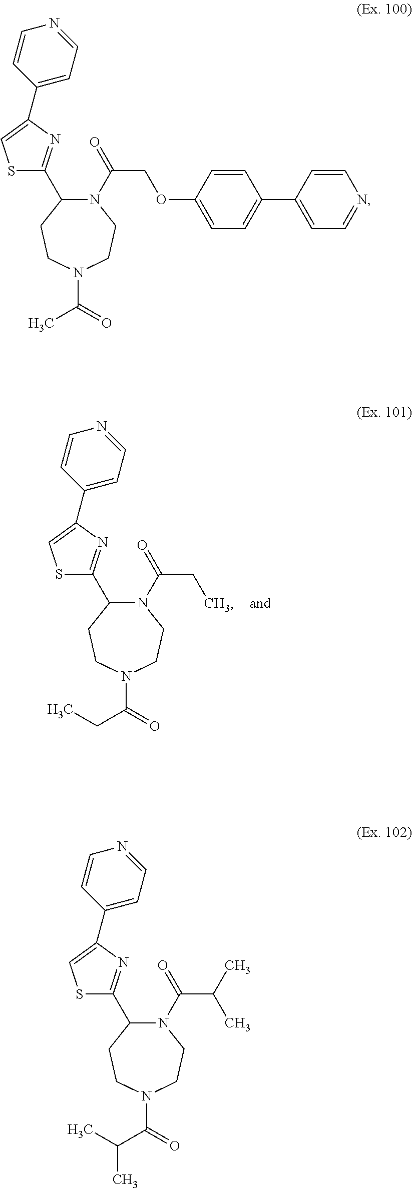 [SMILES: CC(=O)N1CCC(c2nc(-c3ccncc3)cs2)N(C(=O)COc2ccc(-c3ccncc3)cc2)CC1 ; CCC(=O)N1CCC(c2nc(-c3ccncc3)cs2)N(C(=O)CC)CC1 ; CC(C)C(=O)N1CCC(c2nc(-c3ccncc3)cs2)N(C(=O)C(C)C)CC1]

### Processes for the Preparation of Compounds of Formula (I)

In another aspect, the present invention provides novel processes for the preparation of the compounds of formula (I) of the invention.

In one aspect, the present invention relates to a process for the preparation of a compound of the general formula (I) comprising the steps of:
(i) reacting a compound of formula (A)

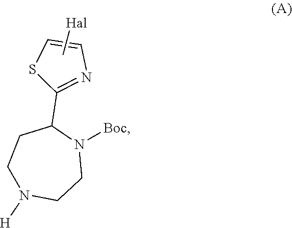

wherein Hal is halogen, preferably Br, and Boc is a tert-butoxycarbonyl protecting group,
with a compound of formula (B)

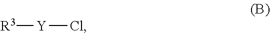

wherein R³ and Y are as defined above,
to obtain a compound of formula (C)

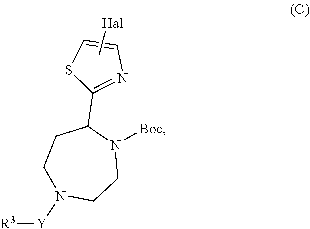

wherein R³, Y, Hal and Boc are as defined above;
(j) reacting the compound of formula (C) with a deprotecting agent to obtain a compound of formula (D)

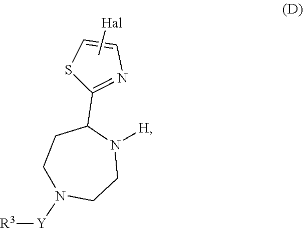

wherein R³, Y and Hal are as defined above;
(k) reacting the compound of formula (D) with a compound of formula (E)

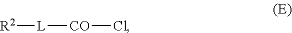

wherein R², and L are as defined above,
to obtain a compound of formula (F)

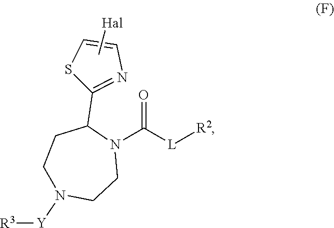

wherein R², R³, L and Y are as defined above;
(l) reacting a compound of formula (F) with a compound of formula (H)

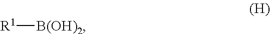

wherein R¹ is as defined above,
if appropriate in the presence of a catalyst to obtain a compound of formula (I)

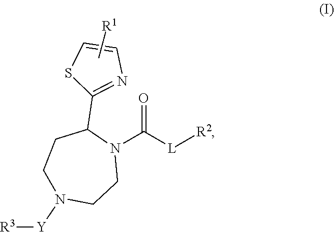

wherein R¹, R², R³, L and Y are as defined above.

An exemplary preparation of preparation of compound of formula (I) is described in Scheme 2 and in the Examples.

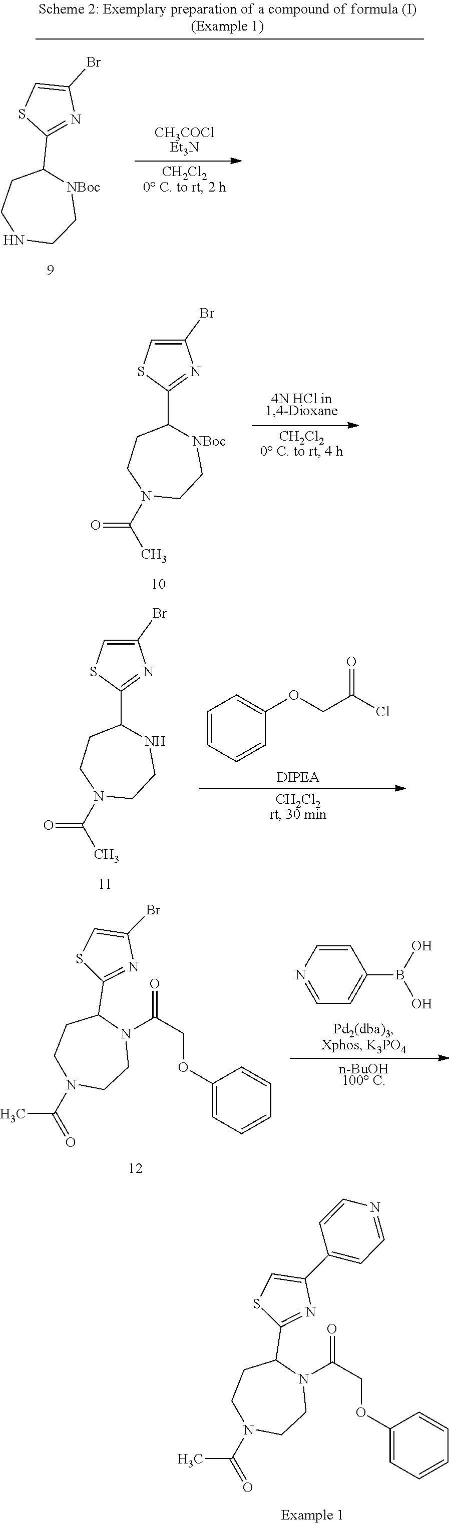

The compounds of formula (A) used as a starting material in process step (i) can be prepared as described in literature procedures or as in the preparation process of the specific Example 9. An exemplary process for the preparation of the compounds of formula (A) is shown in Scheme 1 below:

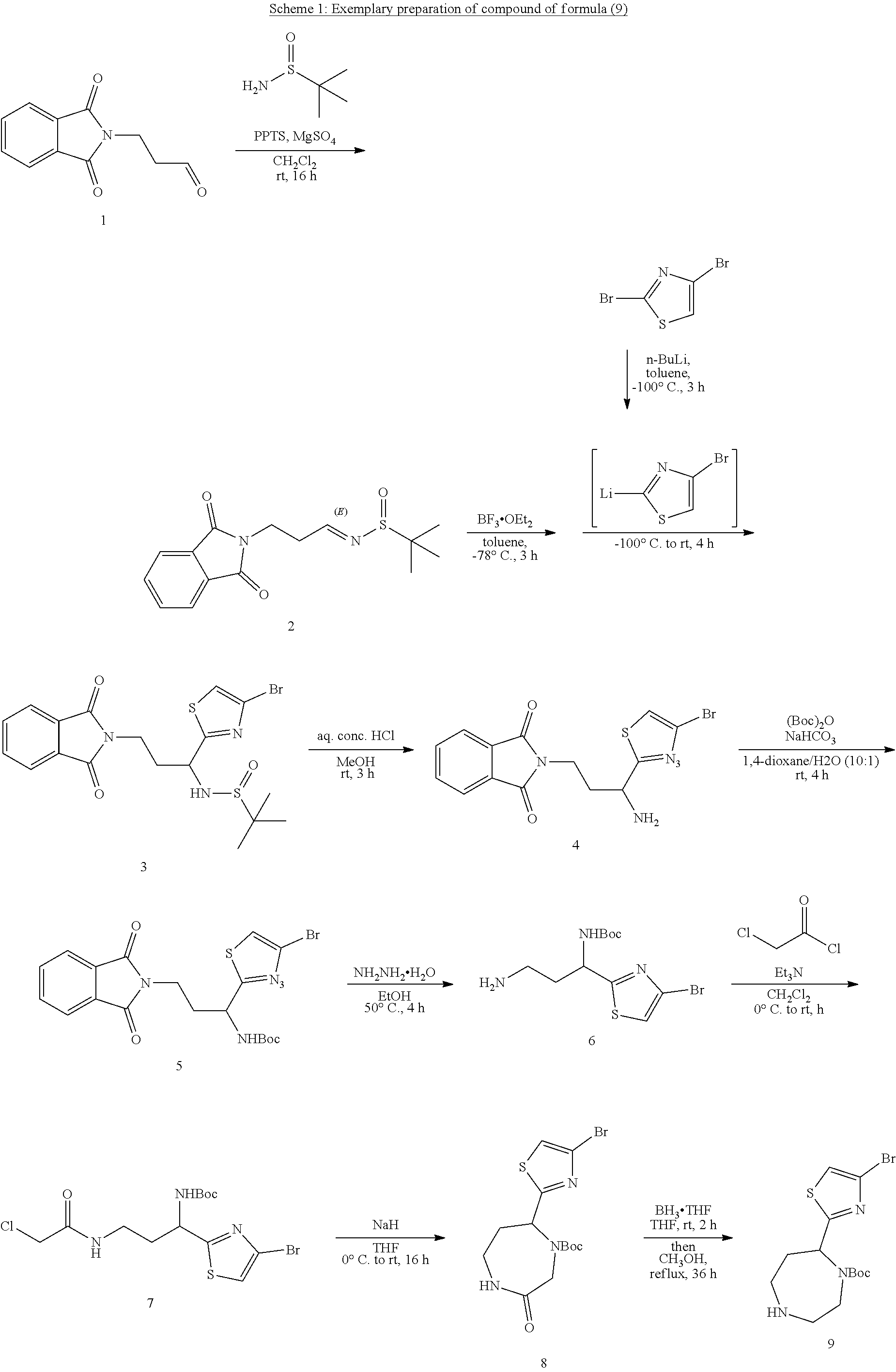

The compounds of formula (B) used as a starting material in process step (i) are commercially available or can be obtained by standard procedures known to the skilled person.

In process step (i), the desired diazepane derivatives of formula (C) may be prepared according to standard N-acetylation procedures known in the art. Further guidance can be found in Scheme 2 and in the Examples disclosed below. Process step (i) is carried out preferably in the present of a solvent and a base, preferably in the presence of dichloromethane and triethylamine.

Process step (j) can be carried out according to standard procedures known in the state of the art for the removal of protecting groups, in particular for removal of the tert-butoxycarbonyl protecting group.

In process step (k), the desired diazepane derivatives of formula (F) may be prepared according to standard N-acetylation procedures known in the art. Further guidance can be found in Scheme 2 and in the Examples disclosed below. Process step (k) is carried out preferably in the present of a solvent and a base, preferably in the presence of dichloromethane and diisopropylethylamine.

The compounds of formula (E) used as a starting material in process step (k) are commercially available or can be obtained by standard procedures known to the skilled person.

Process step (I) can be carried out according to standard procedures known in the state of the art for the C—C-couplings. The compound of formula (I) may be prepared by a standard Suzuki coupling reaction. Further guidance can be found in Scheme 2 and in the Examples disclosed below. Process step (I) is carried out preferably in the present of a solvent and a catalyst.

The compounds of formula (G) used as a starting material in process step (k) are commercially available or can be obtained by standard procedures known to the skilled person.

The compounds of the formulae (C) (D), and (F) are novel. Thus, in another aspect, the invention relates to a compound of the formulae (C), (D) and (F).

In another aspect, the present invention relates to a process for the preparation of a compound of the general formula (I) comprising the steps shown in Scheme 3.

For example, Examples 2, 4, 5, 7, 10, 12, 17, 23, 26, 27, 40, 98 and 99 of Table 1 (see Table 1—Compounds of formula (I) of the Invention) were obtained according to the process of the invention described in Scheme 3. The respective aryl boronic acids Ar—B(OH)₂ used as a starting material are commercially available or can be synthesized according to processes known to the skilled person.

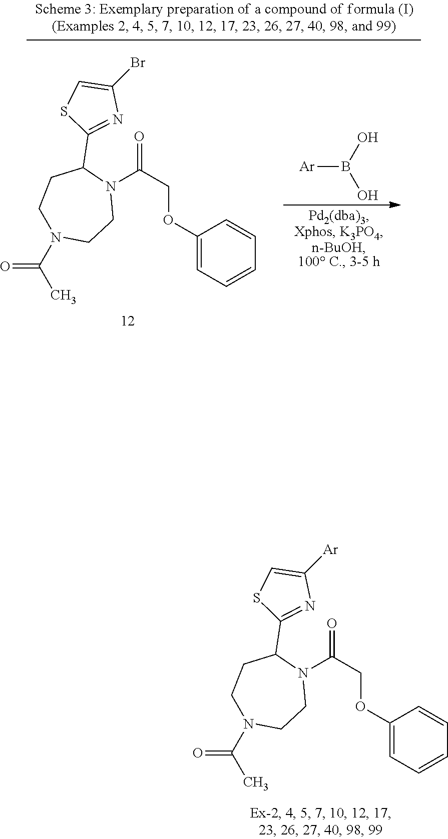

In another aspect, the present invention relates to a process for the preparation of a compound of the general formula (I) comprising the steps shown in Scheme 4.

For example, Examples 41-44, 50, 56, 72, 74, 77, 100-102 of Table 1 (see Table 1—Compounds of formula (I) of the Invention) were obtained according to the process of the invention described in Scheme 4.

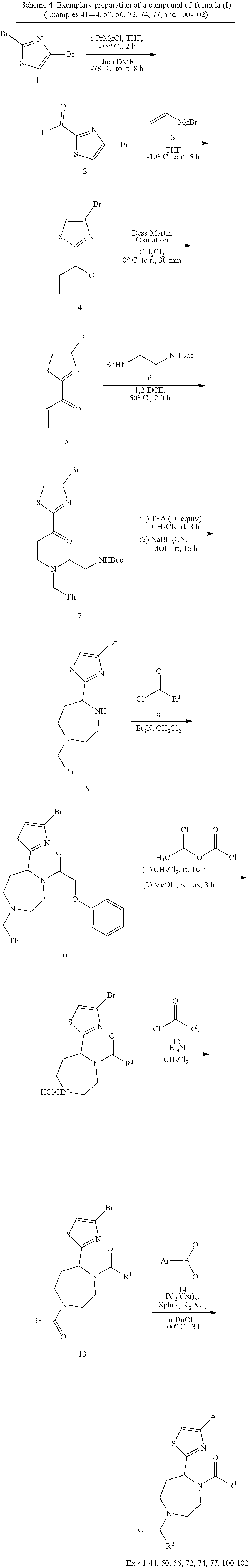

The respective compounds of formulae (9) and (12) and the aryl boronic acids Ar—B(OH)₂ (14) used as a starting material are commercially available or can be synthesized according to processes known to the skilled person.

### Use of the Novel Compounds of Formula (I) as a Medicament

Furthermore, it has been found that the compounds of formula (I) are suitable for use as a medicament. Specifically, it has been found that the compounds of formula (I) can be used in the amelioration, prevention and/or treatment of diseases modulated by CPT-1 inhibitors.

CPT-1 inhibitors previously have been described to have an effect on neurological cells. WO 2009/156479 A1 describes a method to investigate the effect of at least one CPT-I inhibitor in vitro, said method comprising the steps of cultivating cells under conditions essential for cell proliferation, adding of at least one CPT-I inhibitor to the cells, and monitoring the proliferation rate and signal transduction of the cells. Said cells are preferably neuritis/neurons or dendrocytes, more preferred neurons, especially of human origin.

Moreover, WO 2009/156479 A1 describes a method to investigate the effect of at least one CPT-I inhibitor on the neurological status in vivo, said method comprising the steps of administering at least one fatty acid oxidation inhibitor to the neural cells of an affective disorder animal model, and monitoring the neural status, animal functioning. Appropriate animal models are known in the state of the art. Methods for determining the neural status are also known in the art.

The methods of the invention for investigating the effect of the CPT-I inhibitor on the neural status are especially applicable when the concentration and/or amount of the inhibitor in the pharmaceutical composition should be tested

In a further aspect the invention relates to a pharmaceutical composition for treating and/or preventing disorders caused by delipidation of neural tissue, comprising at least one CPT-I inhibitor and at least one excipient and/or auxiliary.

As described in the Examples below, the compounds of formula (I) of the invention were surprisingly and unexpectedly shown to be efficient in the fatty acid uptake assay for activity determination using HEK293 cells in vitro (see Example 4.1), and in vivo in an efficacy study in SOD1 mouse models of ALS (Example 4.2).

Accordingly, the compounds of formula (I) of the invention and their pharmaceutically or veterinarily acceptable salts, hydrates or solvates, exhibit valuable pharmacological properties and are therefore useful as a medicament or as a pharmaceutical. The medicament or pharmaceutical can be further formulated with additional pharmaceutically or veterinary acceptable carriers and/or excipients, e.g. for oral administrations in the form of tablets. Tablets may contain suitable binders, lubricants, disintegrating agents, coloring agents, flavoring agents, flow-inducing agents and/or melting agents, generally known in the art.

Thus, in one aspect, the invention relates to a compound of the general formula (I) as defined herein or a stereoisomer, a pharmaceutically or veterinarily acceptable salt, hydrate or solvate thereof f for use as a medicament.

In another aspect, the invention relates to a pharmaceutical composition comprising a compound of formula (I) as defined herein or a stereoisomer, a pharmaceutically or veterinarily acceptable salt, hydrate or solvate thereof, and a therapeutically inert carrier.

The compounds of formula (I) of the invention exhibit a marked and selective inhibitory effect on the expression and/or activity of the enzyme Carnitine-Palmitoyl-Transferase-1 (CPT-1). This can be determined for example in an vitro fatty acid uptake assay for activity determination and efficacy study (see Example 4.1). The skilled person however may use different assays to determine the direct or indirect inhibition of CPT-1.

Thus, in another aspect, the invention relates to a compound of formula (I) as defined herein for use in the amelioration, prevention or treatment of a disease related to inhibiting the expression and/or activity of the enzyme Carnitine-Palmitoyl-Transferase-1 (CPT-1).

It is already known, that a number of neurological and mental diseases are caused by delipidation of neural tissue and in particular myelin sheets, and that CPT-1 is significantly up-regulated in various tissue from patients suffering from a number of mental an neurological disorders. Disorders, which can be ameliorated, prevented and/or treated with CPT-1 inhibitors are particularly mental and/or neurological disorders.

As mentioned above, it has been found that the compounds of formula (I) of the invention formula (I) or a pharmaceutically or veterinarily acceptable salt, hydrate or solvate thereof, are useful in the amelioration, prevention and/or treatment of diseases caused by or related to delipidation of a neural tissue. Thus, in another aspect, the invention relates to a compound of formula (I) as defined herein for use in the amelioration, prevention and/or treatment of diseases caused by or related to delipidation of a neural tissue.

In one embodiment, the disease is an organic, including symptomatic, mental disorder. This term comprises a range of mental disorders grouped together on the basis of their having in common a demonstrable etiology in cerebral disease, brain injury, or other insult leading to cerebral dysfunction. The dysfunction may be primary, as in diseases, injuries, and insults that affect the brain directly and selectively; or secondary, as in systemic diseases and disorders that attack the brain only as one of the multiple organs or systems of the body that are involved.

In a preferred embodiment, the organic mental disorder may for example be selected from dementia, including dementia in Alzheimer's disease and vascular dementia. In particular preferred embodiments, the disorder is impairment of recent memory and impairment of remote memory.

In another embodiment, the compounds of formula (I) the invention may be used for treating mental and/or behavioral diseases which are due to psychoactive substance use. More specifically diseases resulting from the use of a psychoactive substance such as alcohol, opioids, cannabinoids, cocaine, caffeine, hallucinogens, tobacco, volatile solvents and multiple drug use.

In a further embodiment, the compounds of formula (I) the invention are useful for treating and/or preventing mood disorders, including Manic episode, Bipolar affective disorder, Depression, Depressive episode, Recurrent depressive disorder and Persistent mood disorders such as Cyclothymia and Dysthymia.

In a further embodiment, the diseases are neurotic, stress-related and somatoform diseases, including Phobic anxiety disorders such as Panic disorder, Obsessive-compulsive disorder, Reaction to severe stress and adjustment disorders, Dissociative conversion disorders and Somatoform disorders.

In a further embodiment, the compounds of formula (I) the invention are useful for treating and/or preventing disorders which are behavioral syndromes associated with physiological disturbances and physical factors, including disorders selected from Nonorganic sleep disorders, Sexual dysfunction and Eating disorders such as Anorexia nervosa and Bulimia nervosa.

In a further embodiment, the compounds of formula (I) the invention are useful for treating disorders of adult personality and behavior, such as Paranoid personality disorder, Schizoid personality disorder, Dissocial personality disorder, Emotionally unstable personality disorder, Histrionic personality disorder, Anankastic personality disorder, Anxious personality disorder, Dependent personality disorder, Habit and impulse disorders such as Pathological gambling, Pathological fire-setting, Pathological stealing and Trichotillomania.

In a further embodiment, the compounds of formula (I) the invention are useful for mental retardation, including mild, moderate, severe and profound mental retardation.

In a further embodiment, the compounds of formula (I) the invention are useful for diseases of the nervous system, including the disorders multiple sclerosis and autoimmune neuropathies.

Further disorders which can be treated according to the invention are, for example, Guillian-Barre, encephalomyelitis, Senile plaque, brain tumors i.e. glioblastoma multiforme, Huntingdon disease, Lou Gehrig's disease, pain, chronic pain, myastemia gravis, Sjogren's syndrome, Tourette syndrome, peripheral neuropathy, occipital neuralgia, motor neurone disease, meningitis, Chronic Lyme's disease, Encephalitis, Schilder's disease or diffuse myelinoclastic sclerosis, Chronic Inflammatory Demyelinating Polyneuropathy, Cerebral atrophy, Acute disseminated encephalomyelitis, Attention-deficit hyperactivity disorder, Cataplexy, Fibromyalgia, General anxiety disorder, Hypersexuality, Impulse-control disorders, Narcolepsy, Obsessive-compulsive disorder, Panic disorder, Posttraumatic stress disorder, Premenstrual dysphoric disorder, Social phobia, Chronic pain, Intermittent explosive disorder, Substance abuse and addiction (including alcoholism).

In a further aspect of the invention there is provided a method of preventing and/or treating disorders caused by delipidation of neural tissue, by administering a compound of formula (I) as described herein, to a patient in need thereof in a pharmacologically effective amount.

As used herein, the term “pharmaceutically effective amount” of a CPT-I inhibitor means an amount effective to achieve the desired physiological result, either in cells treated in vitro or in a subject treated in vivo. Specifically, a pharmaceutically effective amount is an amount sufficient to inhibit, for some period of time, one or more clinically defined pathological effects associated with disorders caused by delipidation of neural tissue. The pharmaceutically effective amount may vary depending on the specific CPT-I inhibitor selected, and is also dependent on a variety of factors and conditions related to the subject to be treated and the severity of the disease. For example, if the inhibitor is to be administered in vivo, factors such as age, weight, sex, and general health of the patient as well as dose response curves and toxicity data obtained in pre-clinical animal tests would be among the factors to be considered. If the CPT-I inhibitor is to be contacted with cells in vitro, one would also design a variety of pre-clinical in vitro studies to assess parameters like uptake, half-life, dose, toxicity etc. The determination of a pharmaceutically effective amount for a given agent (inhibitor) is well within the ability of those skilled in the art. Preferably, the inhibitor is present in a concentration of 0.01 to 50% per weight of the pharmaceutical composition, more preferably 1 to 30%.

Administration to an individual or patient may be in a single dose or in repeated doses. Repeated doses are preferred, especially once or twice a day until the symptoms disappear or diminish considerably. The patient to be treated with the methods of the present invention is preferably human. However, also animals, preferably mammals as horses, bovines, dogs or cats and more preferably primates can be treated according to the present invention.

The administration of the compound of formula (I) is not limited to a specific route. Preferred routes of administration to an individual include but are not limited to oral systemic, parenteral, especially dermal, intradermal, intracutaneous, percutaneous, subcutaneous, topical or transdermal application. In this context, a systemic application is an application which results in a distribution of the CPT-I inhibitor throughout the body.

In a preferred embodiment, the invention relates to a compound of formula (I) as defined herein for use in the amelioration, prevention or treatment of a disease related to Morbus Alzheimer, Morbus Parkinson, amyotrophic lateral sclerosis (ALS), inflammatory diseases, acute traumatic events such as surgery or injury, AIDS related wasting due to the toxicity of reverse transcriptase inhibitors, mitochondrial myopathies, senescence and ageing, neuronal ischemia, a polyglutamine disease, dystonia, Leber's heredity optic neuropathy (LHON), schizophrenia, stroke, myodegenerative disorders, Mitochondrial Encephalomyopathy Lactic Acidosis and Strokelike Episodes (MELAS), Myoclonic Epilepsy associated with Ragged-Red Fibers (MERRF), Neuropathy, Ataxia, and Retinitis Pigmentosa (NARP), Progressive External Ophthalmoplegia (PEO), Leigh's disease, Kearns-Sayres Syndromes, muscular dystrophy, myotonic distrophy, chronic fatigue syndrome, Friedreich's Ataxia; developmental delay in cognitive, motor, language, executive function or social skills; epilepsy, peripheral neuropathy, optic neuropathy, autonomic neuropathy, neurogenic bowel dysfunction, sensorineural deafness, neurogenic bladder dysfunction, migraine; renal tubular acidosis, hepatic failure, lactic acidemia, parodontosis, Duchenne muscular dystrophy, Becker's muscular dystrophy, McArdle's disease, abnormities of the testosterone synthesis and/or hypoparathyroidism.

The invention also concerns a method for treating a patient suffering from a mental or neurological disease, preferably form Morbus Alzheimer, Morbus Parkinson, amyotrophic lateral sclerosis (ALS), inflammatory diseases, acute traumatic events such as surgery or injury, AIDS related wasting due to the toxicity of reverse transcriptase inhibitors, mitochondrial myopathies, senescence and ageing, neuronal ischemia, a polyglutamine disease, dystonia, Leber's heredity optic neuropathy (LHON), schizophrenia, stroke, myodegenerative disorders, Mitochondrial Encephalomyopathy Lactic Acidosis and Strokelike Episodes (MELAS), Myoclonic Epilepsy associated with Ragged-Red Fibers (MERRF), Neuropathy, Ataxia, and Retinitis Pigmentosa (NARP), Progressive External Ophthalmoplegia (PEO), Leigh's disease, Kearns-Sayres Syndromes, muscular dystrophy, myotonic distrophy, chronic fatigue syndrome, Friedreich's Ataxia; developmental delay in cognitive, motor, language, executive function or social skills; epilepsy, peripheral neuropathy, optic neuropathy, autonomic neuropathy, neurogenic bowel dysfunction, sensorineural deafness, neurogenic bladder dysfunction, migraine; renal tubular acidosis, hepatic failure, lactic acidemia, parodontosis, Duchenne muscular dystrophy, Becker's muscular dystrophy, McArdle's disease, abnormities of the testosterone synthesis and/or hypoparathyroidism, which comprises the step of administering a therapeutically effective amount of a compound of formula (I) as described herein to a subject, preferably to a human.

In a particular preferred embodiment, the disease caused by or related to delipidation of a neural tissue is amyotrophic lateral sclerosis (ALS).

### EXAMPLES

### Abbreviations and Acronyms

Abbreviations and Acronyms used in the description of the chemistry and in the Examples that follow are:
Boc tert-butoxycarbonyl
CDCl₃ deuterated chloroform
DCM dichloromethane
DIPEA diisopropylethylamine
Ex Example
h hour
¹H-NMR 1H-NMR data of a Compound of formula (I) of the invention
Isolera Flash column chromatography (Make: Isolera)
LCMS LCMS data of a Compound of formula (I) of the invention
Pd₂(dba)₃ tris(dibenzylidenaceton)dipalladium (0)
PPTS pyridinium p-toluene sulfonate
RT room temperature
Structure structure of a Compound of formula (I) of the invention
THF tetrahydrofuran
TLC thin layer chromatography
Xphos 2-dicyclohexylphosphin-2′,4′,6′-triisopropylbiphenyl

### 1. Experimental Procedures

### 1.1 LCMS Method

Ultra-High-Performance Liquid Chromatography (UHPLC) equipped with SQ 6135 (from Agilent)/SQ 2020 (from Shimadzu) Mass spectrometer and

Electro Spray and Atmospheric pressure chemical ionization source (Multimode source with ESI/APCI).
Column: Column Zorbax Eclipse PlusC18 (50×2.1 mm) 1.8 mμ (for Formic Acid Method), or Acquity BEH C18 (2.1×50) mm, 1.7 mμ (for Ammonium Bicarbonate Method)
Flow: 0.800 mL/min or 0.600 mL/min
Eluents: A: H₂O with 0.05% formic acid and B: MeCN or A: H₂O with 10 mM Ammonium Bicarbonate B: MeCN
Gradient: Elution from 5% to 100% B over 2.5 min with an initial hold for 0.5 min and a final hold at 95% B of 1.0 min. Total run time: 4 min.
   The gradient described could be altered in the function of the physico-chemical properties of the compound analyzed and is in no way restrictive.

HPLC-Purity were obtained using Shimadzu Instrument
Column: X-Select C₁₈ (4.6×150 mm, 5 μm) or
X-Bridge column C₈ (4.6×150 mm, 5 μm)
Flow: 0.800 mL/min or 0.600 mL/min
Eluents: A: H₂O with 0.05% formic acid and B: MeCN or
   A: H₂O with 0.05% Ammonium Bicarbonate B: MeCN
Gradient: Elution from 5% to 100% B over 8 min then hold at 5% B of 2 min. Total run time: 10 min.
   The gradient described could be altered in function of the physico-chemical properties of the compound analyzed and is in no way restrictive.

### 1.2 NMR Methods

Proton (¹H) nuclear magnetic resonance (NMR) spectras are measured with an Avance Neo Nanobay (400 MHZ) spectrometer with residual protonated solvent (CDCl₃ δ 7.28; CD₃OD δ 3.31 and DMSO δ 2.50) as standard. The NMR data of the synthesized examples are in agreement with their corresponding structural assignments.

### 2. Process for the Preparation of the Compounds of Formula (I)

An exemplary synthesis of a compound of formula (I) is described below in Schemes 1 and 2. The compounds of formula (I) of the invention can be obtained according to the process described in Schemes 1 and 2.

The starting materials are either commercially available or are prepared in similar manners as described in literature procedures or in the specific example.

It should be apparent to those skilled in the art that the sequence of the synthetic steps is dependent on starting materials availability and functional group compatibility and could vary from compound to compound.

### 3. Examples of the Compounds of Formula (I) of the Invention

The following Examples are merely specific embodiments of the present invention and are intended to illustrate but not to limit the invention.

### 3.1 Preparation of Intermediates for the Preparation of Compounds of Formula (I)

### 3.1.1 Preparation of tert-butyl 7-(4-bromothiazol-2-yl)-1,4-diazepane-1-carboxylate (9)

Tert-butyl 7-(4-bromothiazol-2-yl)-1,4-diazepane-1-carboxylate (9) was obtained according to the process described in Scheme 1.

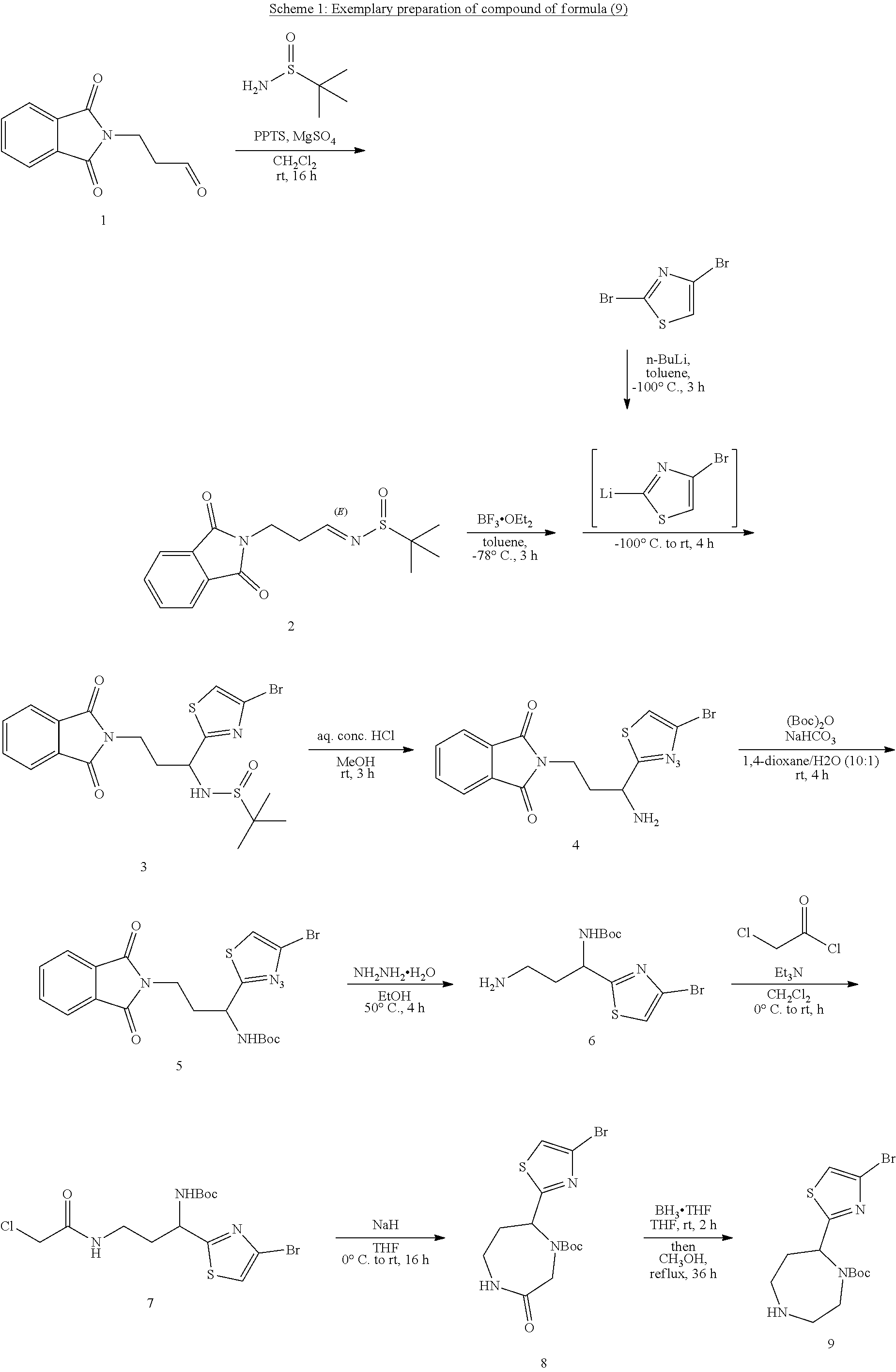

### Step (a): Preparation of (E)-N-(3-(1,3-dioxoisoindolin-2-yl)propylidene)-2-methylpropane-2-sulfinamide (2)

To a stirred solution of 3-(1,3-dioxoisoindolin-2-yl)propanal (1) (14 g, 68.9 mmol) in anhydrous DCM (200 mL) 2-methylpropane-2-sulfinamide (9.1 g, 75.8 mmol) was added at RT. PPTS (0.86 g, 3.4 mmol) and anhydrous magnesium sulfate (41 g, 344 mmol) were added and the mixture was stirred at RT for 16 h. The reaction was monitored by TLC. The mixture was filtered through celite. The filtrate was concentrated to get the crude which was purified by silica column chromatography using Isolera by eluting with 30% ethyl acetate in pet. ether to afford (E)-N-(3-(1,3-dioxoisoindolin-2-yl)propylidene)-2-methylpropane-2-sulfinamide (2) (15 g, yield: 71%). 1H-NMR (400 MHZ, CDCl₃): δ 8.14 (t, J=3.60 Hz, 1H), 7.89-7.87 (m, 2H), 7.77-7.74 (m, 2H), 4.11-4.02 (m, 2H), 3.01-2.96 (m, 2H), 1.19 (s, 9H), LCMS: 307 (M+1).

### Step (b): Preparation of: 2-(3-amino-3-(4-bromothiazol-2-yl) propyl) isoindoline-1,3-dione (3)

To a stirred solution of 2,4-dibromothiazole (23.7 g, 97.97 mmol) in anhydrous toluene (150 mL) n-BuLi (1.6 M in THF, 61.2 mL, 97.97 mmol) was added dropwise at −100° C. The mixture was stirred with a mechanical stirrer at the same temperature for 3 h.

In an another set up, to a solution of (E)-N-(3-(1,3-dioxoisoindolin-2-yl) propylidene)-2-methylpropane-2-sulfinamide (2) (15 g, 48.98 mmol) in anhydrous toluene (100 mL) boron trifluoride diethyl etherate (13.8 mL) was added dropwise at −78° C. and stirred for 3 h. After 3 h, this mixture was cannulated dropwise into the above mixture containing 2,4-dibromothiazole and n-butyl lithium at −100° C. The resulting mixture was allowed to slowly warm to RT and stirred for 4 h. The reaction was monitored by TLC. The reaction was cooled to 0° C. then quenched by the slow addition of ice-cooled water (100 mL). The mixture was extracted with ethyl acetate (2×500 mL). The combined organic layer was dried over anhydrous sodium sulphate, filtered and concentrated. The crude was purified by silica column chromatography using Isolera by eluting with 40% ethyl acetate in pet. ether to afford 8 g of (67% pure by LCMS) N-(1-(4-bromothiazol-2-yl)-3-(1,3-dioxoisoindolin-2-yl)propyl)-2-methylpropane-2-sulfinamide (3). 1H-NMR (400 MHZ, CDCl₃): δ 7.77-7.75 (m, 2H), 7.70-7.68 (m, 2H), 6.85 (s, 1H), 5.18 (d, J=9.60 Hz, 1H), 4.88-4.84 (m, 1H), 4.07-3.99 (m, 1H), 3.96-3.90 (m, 1H), 2.98-2.90 (m, 1H), 2.52-2.45 (m, 1H), 1.41 (s, 9H). LCMS: 470 & 472 (M+1).

### Step (c): Preparation of 2-(3-amino-3-(4-bromothiazol-2-yl)propyl)isoindoline-1,3-dione (4)

To a solution N-(1-(4-bromothiazol-2-yl)-3-(1,3-dioxoisoindolin-2-yl)propyl)-2-methylpropane-2-sulfinamide (3) (8 g) in MeOH (50 mL) conc. HCl (8 mL) was added, and the mixture was stirred at RT for 3 h. The reaction was monitored by TLC. The mixture was concentrated under reduced pressure to afford 7 g of 2-(3-amino-3-(4-bromothiazol-2-yl)propyl)isoindoline-1,3-dione (4).

### Step (d): Preparation of tert-butyl (1-(4-bromothiazol-2-yl)-3-(1,3-dioxoisoindolin-2-yl)propyl)carbamate (5)

To a stirred solution of 2-(3-amino-3-(4-bromothiazol-2-yl)propyl)isoindoline-1,3-dione (4) (5 g, 13.66 mmol) in dioxane/water (55 mL, 10:1) sodium bicarbonate (7.1 g, 68.3 mmol) was added at RT. Di-tert-butyl dicarbonate (5.9 mL, 27.3 mmol) was added and the mixture was stirred at RT for 4 h. The reaction was monitored by TLC. The mixture was diluted with water (100 mL) and extracted with ethyl acetate (3×100 mL). The combined organic layer was dried over anhydrous sodium sulphate, filtered and concentrated. The crude was purified by silica column chromatography using Isolera by eluting with 15% to 20% ethyl acetate in pet. ether to afford tert-butyl (1-(4-bromothiazol-2-yl)-3-(1,3-dioxoisoindolin-2-yl)propyl)carbamate (5) (2.4 g, yield: 38%). 1H-NMR (400 MHZ, CDCl₃): δ 7.84-7.82 (m, 2H), 7.75-7.71 (m, 2H), 7.04 (s, 1H), 5.60-5.58 (m, 1H), 5.15-5.13 (m, 1H), 3.90-3.85 (m, 2H), 2.46-2.45 (m, 2H), 1.48 (s, 9H), LCMS: 466 & 468 (M+1).

### Step (e): Preparation of tert-butyl (3-amino-1-(4-bromothiazol-2-yl)propyl)carbamate (6)

To a stirred solution of tert-butyl (1-(4-bromothiazol-2-yl)-3-(1,3-dioxoisoindolin-2-yl)propyl)carbamate (5) (2.4 g, 5.15 mmol) in ethanol (20 mL) hydrazine hydrate (0.5 mL, 10.3 mmol) was added. The mixture was heated at 50° C. for 4 h. The reaction was monitored by TLC and the solid was filtered through celite and the filtrate was concentrated. The crude was purified by silica column chromatography using Isolera by eluting with 10% MeOH in DCM to afford tert-butyl (3-amino-1-(4-bromothiazol-2-yl)propyl)carbamate (6) (1.5 g, 81% pure). LCMS: 336 & 338 (M+1).

### Step (f): Preparation of tert-butyl (1-(4-bromothiazol-2-yl)-3-(2-chloroacetamido)propyl)carbamate (7)

To a stirred solution of tert-butyl (3-amino-1-(4-bromothiazol-2-yl)propyl)carbamate (6) (1.5 g, 4.46 mmol) in anhydrous DCM (20 mL) triethylamine (0.9 mL, 6.69 mmol) was added at 0° C. Chloroacetyl chloride (0.53 mL, 6.69 mmol) was added dropwise to the mixture and stirred for 4 h. The reaction was monitored by TLC and the mixture was diluted water (50 mL) and DCM (100 mL). The organic layer was separated and washed with 10% aqueous solution of sodium bicarbonate (50 mL×2) and brine (50 mL). The organic layer was dried over anhydrous sodium sulphate, filtered and concentrated to afford tert-butyl (1-(4-bromothiazol-2-yl)-3-(2-chloroacetamido)propyl)carbamate (7) (1.7 g crude). LCMS: 412 & 414 (M+1).

### Step (g): Preparation of tert-butyl 7-(4-bromothiazol-2-yl)-3-oxo-1,4-diazepane-1-carboxylate (8)

To an ice-cooled solution of tert-butyl (1-(4-bromothiazol-2-yl)-3-(2-chloroacetamido)propyl)carbamate (7) (1.7 g, 4.12 mmol) in anhydrous THF (200 mL) sodium hydride (60% dispersion in mineral oil, 0.95 g, 23.8 mmol) was added in three portions and the mixture was stirred at RT for 16 h. The reaction was quenched with ice-cooled water (200 mL) and extracted with ethyl acetate (3×100 mL). The combined organic layer was dried over anhydrous sodium sulphate, filtered and concentrated. The crude was purified by silica column chromatography using Isolera by eluting with 65% ethyl acetate in pet. ether to afford tert-butyl 7-(4-bromothiazol-2-yl)-3-oxo-1,4-diazepane-1-carboxylate (8) (0.2 g, yield: 13%). LCMS: 322 (M+1, t-butyl cleaved).

### Step (h): Preparation of tert-butyl 7-(4-bromothiazol-2-yl)-1,4-diazepane-1-carboxylate (9)

To an ice-cooled solution of tert-butyl 7-(4-bromothiazol-2-yl)-3-oxo-1,4-diazepane-1-carboxylate (8) (0.2 g, 0.531 mmol) in anhydrous THF (5 mL) a 1M solution of BH₃. THF (1.59 mL, 1.59 mmol) was added. The mixture was stirred at RT for 2 h. The reaction was quenched with water (5 mL) and methanol (5 mL). The mixture was heated at 70° C. for 36 h for cleaving the borane complex. The mixture was diluted ice-cooled water (10 mL) and extracted with DCM (2×10 mL). The combined organic layer was dried over anhydrous sodium sulphate, filtered and concentrated to afford tert-butyl 7-(4-bromothiazol-2-yl)-1,4-diazepane-1-carboxylate (9) (0.18 g, yield: 93%). LCMS: 362 & 364 (M+1).

### 3.2 Preparation of Compounds of Formula (I)

Example 1 was obtained according to the process described in Scheme 2.

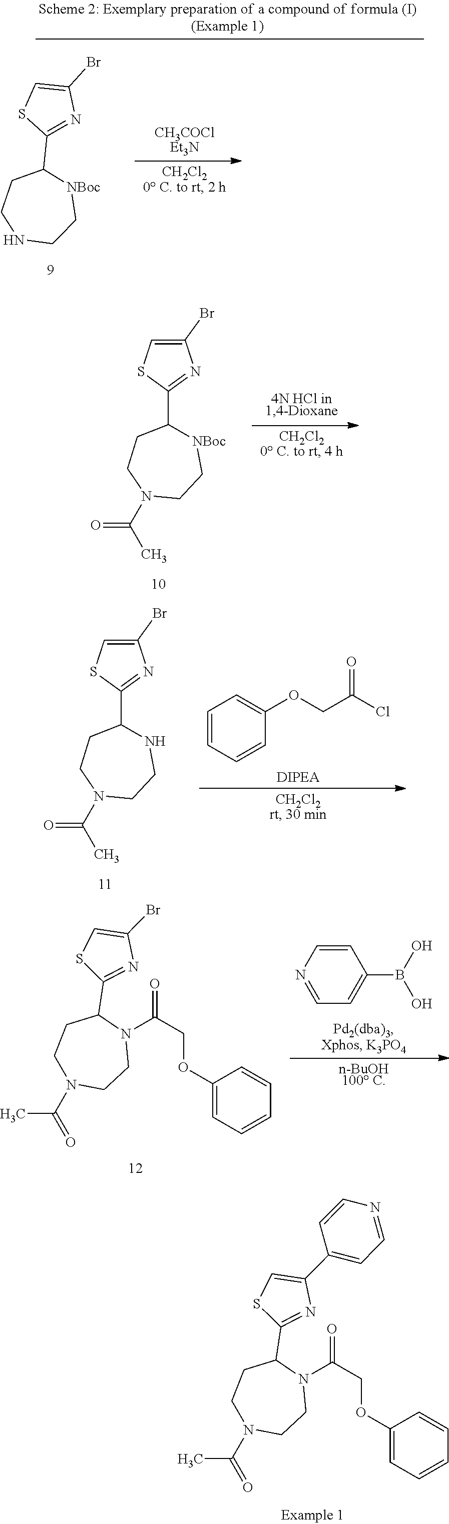

### Step (i): Preparation of tert-butyl 4-acetyl-7-(4-bromothiazol-2-yl)-1,4-diazepane-1-carboxylate (10)

To a stirred solution of tert-butyl 7-(4-bromothiazol-2-yl)-1,4-diazepane-1-carboxylate (9) (0.18 g, 0.497 mmol) in DCM (5 mL) triethylamine (0.2 mL, 1.49 mmol) was added. Acetyl chloride (0.047 g, 0.596 mmol) was added at 0° C. and the mixture was stirred at RT for 2 h. The reaction was monitored by TLC and then quenched with water (10 mL) and extracted with DCM (2×10 mL). The combined organic layer was dried over anhydrous sodium sulphate, filtered and concentrated. The crude was purified by neutral alumina column chromatography using Isolera by eluting with 40% ethyl acetate in pet. ether to afford tert-butyl 4-acetyl-7-(4-bromothiazol-2-yl)-1,4-diazepane-1-carboxylate (10) (0.11 g, 63% pure). LCMS: 404 & 406 (M+1).

### Step (j): Preparation of 1-(5-(4-bromothiazol-2-yl)-1,4-diazepan-1-yl)ethan-1-one (11)

To a solution of tert-butyl 4-acetyl-7-(4-bromothiazol-2-yl)-1,4-diazepane-1-carboxylate (10) (0.11 g, 0.272 mmol) in anhydrous DCM (5 mL) 4N HCl in dioxane (0.14 mL, 0.54 mmol) was added at 0° C. The mixture was stirred at RT for 4 h and then concentrated under reduced pressure to afford 1-(5-(4-bromothiazol-2-yl)-1,4-diazepan-1-yl)ethan-1-one (11) (0.09 g, 75% pure). LCMS: 304 & 306 (M+1).

### Step (k): Preparation of 1-(4-acetyl-7-(4-bromothiazol-2-yl)-1,4-diazepan-1-yl)-2-phenoxyethan-1-one (12)

To an ice-cooled solution of 1-(5-(4-bromothiazol-2-yl)-1,4-diazepan-1-yl)ethan-1-one (11) (0.09 g, 0.264 mmol) in DCM (5 mL) DIPEA (0.13 mL, 0.738 mmol) was added. Phenoxyacetyl chloride (46 mg, 0.27 mmol) was added and the mixture was stirred at RT for 30 min. The reaction was monitored by TLC. The mixture was diluted with DCM (15 mL), washed with water (2×10 mL), brine (10 mL) then dried over anhydrous sodium sulphate, filtered and concentrated. The crude was purified by silica column chromatography using Isolera by eluting with 7% MeOH in DCM to afford 1-(4-acetyl-7-(4-bromothiazol-2-yl)-1,4-diazepan-1-yl)-2-phenoxyethan-1-one (12) (0.09 g, yield: 75%). LCMS: 438 & 440 (M+1).

### Step (I): Preparation of 1-(4-acetyl-7-(4-(pyridin-4-yl)thiazol-2-yl)-1,4-diazepan-1-yl)-2-phenoxyethan-1-one (Example 1)

To a solution of 1-(4-acetyl-7-(4-bromothiazol-2-yl)-1,4-diazepan-1-yl)-2-phenoxyethan-1-one (12) (90 mg, 0.205 mmol) in n-butanol (10 mL) potassium phosphate (87 mg, 0.410 mmol) was added. The mixture was degassed with nitrogen for 10 min. Pyridin-4-ylboronic acid (38 mg, 0.308 mmol), Xphos (10 mg, 0.0205 mmol) and Pd₂(dba)₃ (10 mg, 0.01 mmol) were added and the mixture was degassed with nitrogen for another 10 min then heated to 100° C. for 3 h. The reaction was monitored by TLC and the mixture was filtered through celite and the filtrate was concentrated. The crude was purified by silica column chromatography using Isolera by eluting with 6% MeOH in DCM to afford 1-(4-acetyl-7-(4-(pyridin-4-yl)thiazol-2-yl)-1,4-diazepan-1-yl)-2-phenoxyethan-1-one (Example 1) (0.08 g, yield: 89%). 1H-NMR (400 MHZ, MeOH-d₄): δ 8.64 (d, J=4.40 Hz, 2H), 8.32 (br s, 1H), 7.87 (d, J=4.80 Hz, 2H), 7.28-7.26 (m, 2H), 6.98-6.94 (m, 3H), 6.10-5.68 (m, 1H), 5.02-4.94 (m, 2H), 4.40-4.05 (m, 3H), 3.80-3.60 (m, 2H), 3.40-3.20 (m, 1H), 2.85-2.70 (m, 2H), 2.35-2.10 (m, 3H). LCMS: 437 (M+1).

Examples 2, 4, 5, 7, 10, 12, 17, 23, 26, 27, 40, 98 and 99 (see Table 1—Compounds of formula (I) of the Invention) were synthesized according to the general process of the invention shown in Scheme 3 and as described below.

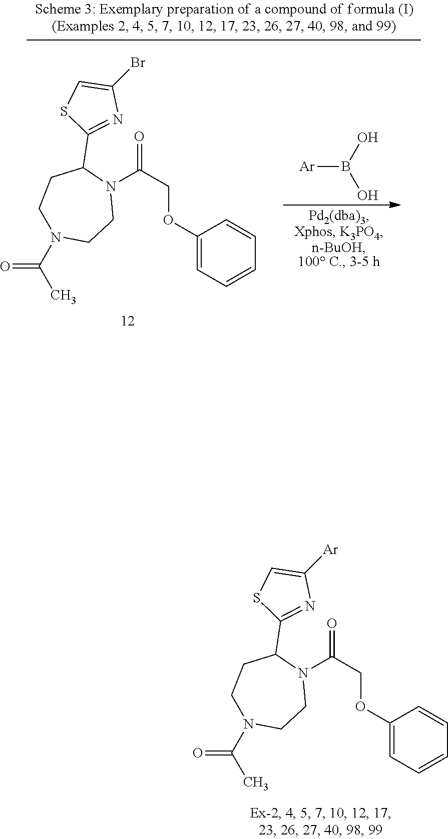

To a stirred solution of bromothiazole compound 12 (1.0 eq) obtained according to step (k) in n-butanol under nitrogen atmosphere were added potassium phosphate tribasic (3.0 eq), the respective aryl boronic acid (1.2 eq), Xphos (10 mol %). The reaction mixture was degassed with nitrogen for 10 min. To this solution, Pd₂(dba)₃ (10 mol %) was added and heated at 100° C. for 3-5 h. The reaction progress was monitored by TLC. Upon completion of the reaction, reaction mixture was diluted with water (20 ml) and extracted with CH₂Cl₂ (50 ml×3). The combined organic extract was dried over anhydrous Na₂SO₄, filtered and concentrated to get crude. The crude was purified by flash column chromatography (silica-gel, 100-200 mesh) using MeOH/CH₂Cl₂ (0-3%) as an eluent to obtain the compounds of Examples 2, 4, 5, 7, 10, 12, 17, 23, 26, 27, 40, 98, and 99. In some cases, both the enantiomers were separated on chiral SFC using LUX-C₄ column where the mobile phase used was isopropyl alcohol and liquid carbon dioxide.

Examples 41-44, 50, 56, 72, 74, 77, 100-102 (see Table 1—Compounds of formula (I) of the Invention) were synthesized according to the general process of the invention shown in Scheme 4 and as described below.

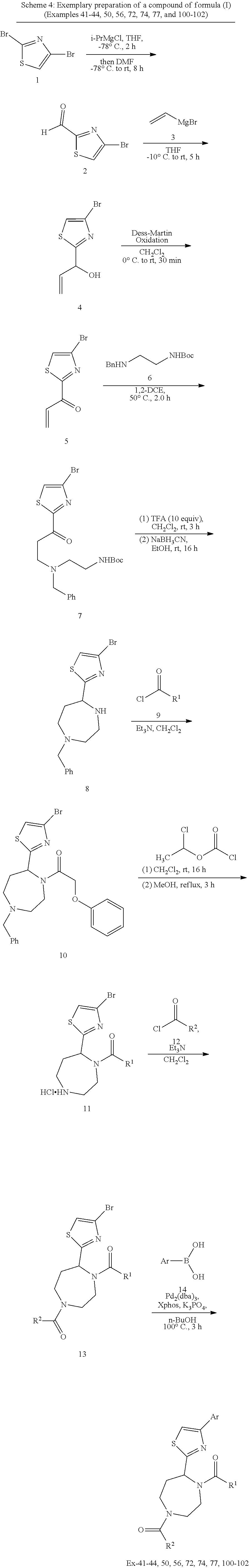

### Step (a): Preparation of 4-bromothiazole-2-carbaldehyde (2)

To solution of 2,4-dibromothiazole (1) (20 g, 82 mmol) in THF (200 ml) was added isopropylmagnesium chloride (49.4 ml, 99 mmol) dropwise at −78° C. and the reaction mixture was stirred for 2 h at the same temperature. To this reaction mixture at −78° C., DMF (37.6 g, 515 mmol) was added dropwise and then the reaction mixture slowly warmed to room temperature and stirred for 8 h. The reaction mixture was quenched with aq. NH₄Cl and extracted in CH₂Cl₂ (500 mL×3). The combined organic extract was washed with water (100 mL), brine solution (100 mL), dried over anhydrous Na₂SO₄, filtered and evaporated under vacuum to get crude. The crude was purified by flash column chromatography (silica-gel, 100-200 mesh) using ethyl acetate/hexanes (0-20%) as an eluent to afford 4-bromothiazole-2-carbaldehyde (2) (9.6 g, 50.0 mmol, 60% yield) as a light-yellow solid. 1H NMR (400 MHZ, CDCl₃): 7.69 (d, J=1.2 Hz, 1H), 9.96 (d, J=1.2 Hz, 1H).

### Step (b): Preparation of 1-(4-bromothiazol-2-yl)prop-2-en-1-ol (4)

Vinylmagnesium bromide (21.87 ml, 1.0 M in THF, 21.87 mmol) was added drop-wise to the stirred solution of 4-bromothiazole-2-carbaldehyde (2) (3.5 g, 18.23 mmol) in THF (30 ml) at −10° C., and the reaction mixture was slowly brought to room temperature and stirred for 5 h. The progress of the reaction was monitored by TLC. After completion of the reaction as indicated by TLC, the reaction mixture was quenched with aq. NH₄Cl. The reaction mixture was diluted with EtOAc and layers were separated. The aqueous layer was extracted with EtOAc (50 mL×2). The combined organic extract was washed with water (50 mL), brine solution (50 mL), dried over anhydrous Na₂SO₄. filtered and evaporated under vacuum to get crude. The crude was purified by flash column chromatography (silica-gel, 230-400 mesh) using ethyl acetate/hexanes (0-20%) as an eluent to afford 1-(4-bromothiazol-2-yl)prop-2-en-1-ol (4) (2 g, 9.09 mmol, 50% yield) as a yellow gummy liquid. 1H-NMR (400 MHZ, CDCl₃): δ 7.24 (s, 1H), 6.15-6.13 (m, 1H), 5.51-5.49 (m, 2H), 4.94 (d, J=6.40 Hz, 1H), 3.22 (s, 1H).

### Step (c): Preparation of 1-(4-bromothiazol-2-yl)prop-2-en-1-one (5)

To a stirred solution of 1-(4-bromothiazol-2-yl)prop-2-en-1-ol (4) (2 g, 9.09 mmol) in CH₂Cl₂ (20 ml) was added Dess-Martin Periodinane (4.63 g, 10.90 mmol) at 0° C. under inert atmosphere. The reaction mixture was allowed to warm to room temperature and stirred for 30 min. The progress of the reaction was monitored by TLC. After completion of the reaction, the reaction mixture was quenched with saturated sodium thiosulfate solution. The reaction mixture was diluted with EtOAc and layers were separated. The aqueous layer was extracted again in EtOAc (50 mL×2). The combined organic extract was washed with water (50 mL), brine solution (50 mL), dried over anhydrous Na₂SO₄ and evaporated under vacuum to get crude. The crude was purified by flash column chromatography (silica-gel, 230-400 mesh) using ethyl acetate/hexanes (0-10%) as an eluent to afford 1-(4-bromothiazol-2-yl)prop-2-en-1-one (5) (1.2 g, 5.50 mmol, 60% yield) as an off white solid. 1H-NMR (400 MHZ, CDCl₃): δ 7.63 (s, 1H), 7.52 (dd, J=10.40 Hz, J=17.60 Hz, 1H), 6.76 (dd, J=17.60 Hz, J=1.60 Hz, 1H), 6.07 (dd, J=10.40 Hz, J=1.60 Hz, 1H).

### Step (d): Preparation of tert-butyl (2-(benzyl(3-(4-bromothiazol-2-yl)-3-oxopropyl)amino)ethyl)carbamate (7)

To a stirred solution of 1-(4-bromothiazol-2-yl)prop-2-en-1-one (5) (3.0 g, 13.76 mmol) in 1,2-DCE (3 mL) was added tert-butyl (2-(benzylamino)ethyl)carbamate (3.44 g, 13.76 mmol) and stirred at 50° C. for 2 h. The solvent was evaporated to give tert-butyl (2-(benzyl(3-(4-bromothiazol-2-yl)-3-oxopropyl)amino)ethyl)-carbamate (7) in quantitative yield. 1H-NMR (400 MHZ, CDCl₃): δ 7.57 (s, 1H), 7.40-7.20 (m, 5H), 4.91 (s, 1H), 3.65 (s, 2H), 3.30 (t, J=6.8 Hz, 2H), 3.20 (s, 2H), 3.02 (t, J=6.8, 2H), 2.62-2.59 (m, 2H), 1.45 (s, 9H).

### Step (e): Preparation of 2-(1-benzyl-1,4-diazepan-5-yl)-4-bromothiazole (8)

To a stirred solution of tert-butyl (2-(benzyl(3-(4-bromothiazol-2-yl)-3-oxopropyl)amino)ethyl)carbamate (7) (8 g, 17.08 mmol) in CH₂Cl₂ (80 ml) was added 2,2,2-trifluoroacetic acid (19.60 ml, 256 mmol) and the resulting reaction mixture was stirred at room temperature for 3 h. Then, the reaction mixture was concentrated under reduced pressure to get the residue. To this residue, EtOH (80 ml) was added and stirred for 2 h. To this stirring solution, NaCNBH₃ (4.29 g, 68.3 mmol) was added portion wise and the resulting mixture was stirred at room temperature for 16 h. The solvents were evaporated and the residue was dissolved in CH₂Cl₂ (100 mL). To this solution, 2 M NaOH_{aq} (30 mL) was added and stirred for 20 min. The layers were separated, aqueous layer was extracted with CH₂Cl₂ (50 mL×2), then, the combined organic extract was dried over anhydrous Na₂SO₄, filtered and concentrated under reduced pressure to give the crude. The crude was purified by flash column chromatography (silica-gel, 100-200 mesh) using CH₂Cl₂/MeOH (0-5%) as an eluent to afford 2-(1-benzyl-1,4-diazepan-5-yl)-4-bromothiazole (8) (3.1 g, 8.80 mmol, 51% yield) as an off-white solid. LCMS (ESI, +ve) m/z 352.20 [M+H]⁺.

### Step (f): Synthesis of Compound 10 of Scheme 4

To a stirred solution of tert-butyl (2-(benzyl(3-(4-bromothiazol-2-yl)-3-oxopropyl)amino)ethyl) carbamate (8) (1.0 eq.) in CH₂Cl₂ was added triethyamine (3.0 eq.) and the reaction mixture cooled to 0° C. To this solution acid chloride 9 (1.2 eq.) was added drop wise. The resulting reaction mixture was allowed to warm to room temperature and stirred for 30 min. The completion of the reaction was monitored by TLC. Upon completion of the reaction, the reaction mixture was diluted with CH₂Cl₂ (50 mL×2), washed with water (20 mL), brine (20 mL) then dried over anhydrous Na₂SO₄, filtered and concentrated to get crude. The crude was purified by flash column chromatography (silica-gel, 100-200 mesh) using CH₂Cl₂/MeOH (0-5%) as an eluent to obtain N-benzyl-homopiperazine derivative (10).

### Step (g): Synthesis of Compound 11 of Scheme 4

To a stirred solution of N-benzyl-homopiperazine derivative (10) (1.0 eq.) in CH₂Cl₂ was added 1-chloroethyl carbonochloridate (3.0 eq.) at room temperature and stirred for 16 h. After this time, volatiles were removed under rotary evaporator and to this crude material was added MeOH and refluxed for 3 h. Solvents were evaporated and the residue was re-dissolved in CH₂Cl₂ and evaporated. Again added 5-10 mL of CH₂Cl₂ and sonicated for 5 min, at this point, a white precipitate was formed, which was filtered, washed with CH₂Cl₂ to obtain crude of homopiperazine hydrochloride derivative 11 as a solid, which was as such used in the next step without further purification.

### Step (h): Synthesis of Compound 13 of Scheme 4

To a stirred solution of homopiperazine hydrochloride derivative 11 (1.0 eq.) in CH₂Cl₂ was added triethylamine (5.0 eq.) and the reaction mixture was cooled to 0° C. To this reaction mixture, the respective acetyl chloride 12 (1.2 eq.) was added and the resulting reaction mixture was allowed to warm to room temperature and stirred for 1 h. After completion of the reaction, reaction mixture was diluted with water (10 ml) and extracted with CH₂Cl₂ (20 ml×3). The combined organic layer was dried over anhydrous Na₂SO₄, filtered and concentrated to get crude product. The crude was purified by flash column chromatography (silica-gel, 100-200 mesh) using MeOH/CH₂Cl₂ (0-3%) as an eluent to afford compound 13.

### Step (i): Synthesis of a Compound of the Invention, i.e. of Examples 41-44, 50, 56, 72, 74, 77, and 100-102

To a stirred solution of compound 13 (1.0 eq) in n-butanol under nitrogen atmosphere were added potassium phosphate tribasic (3.0 eq), the respective aryl boronic acid (14) (1.2 eq), Xphos (10 mol %). The reaction mixture was degassed with nitrogen for 10 min. To this solution, Pd₂(dba)₃ (10 mol %) was added and heated at 100° ° C. for 3-5 h. The reaction progress was monitored by TLC. Upon completion of the reaction, reaction mixture was diluted with water (20 ml) and extracted with CH₂Cl₂ (50 ml×3). The combined organic extract was dried over anhydrous Na₂SO₄, filtered and concentrated to get crude. The crude was purified by flash column chromatography (silica-gel, 100-200 mesh) using MeOH/CH₂Cl₂ (0-3%) as an eluent to provide corresponding products.

### 3.2 Examples 2 to 102

The compounds exemplifying the invention are described in Table 1.

The compounds described in Table 1 can be obtained according to the processes described in Schemes 1, 2, 3 and 4.

TABLE 1
Compounds of formula (I) of the invention

| Ex | Structure | ¹H-NMR | MS |
|---|---|---|---|
| 1 | 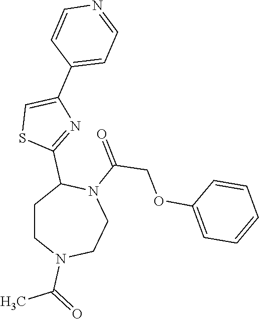 [SMILES: CC(=O)N1CCC(c2nc(-c3ccncc3)cs2)N(C(=O)COc2ccccc2)CC1] | ¹H NMR (400 MHz, MeOH-d₄) δ 2.09 (s, 0.80H), 2.14 (s, 0.52H), 2.16 (s, 0.65H), 2.20 (s, 0.95H), 2.41-2.83 (m, 2H), 3.12-3.27 (m, 0.5H), 3.48-3.77 (m, 2.5H), 3.92-4.43 (m, 3H), 4.93-5.06 (m, 2H), 5.70-5.84 (m, 0.5H), 5.94-6.07 (m, 0.5H), 6.87-7.05 (m, 3H), 7.21-7.34 (m, 2H), 7.95- 8.00 (m, 2H), 8.19 (s, 0.23H), 8.20 (s, 0.30H), 8.27 (s, 0.15H), 8.27 (s, 0.25H), 8.56-8.63 (m, 2H). Enantiomer 1: SFC-Chiral purity = 99.3%, RT = 7.91 min; Enantiomer 2: SFC-Chiral purity = 99.3%, RT = 10.52 min | 437.2 (M+1) |
| 2 | 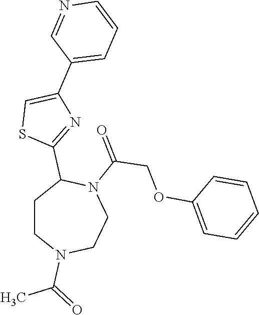 [SMILES: CC(=O)N1CCC(c2nc(-c3cccnc3)cs2)N(C(=O)COc2ccccc2)CC1] | ¹H NMR (400 MHz, MeOH-d₄) δ 2.08 (s, 0.85H), 2.13 (s, 0.56H), 2.14 (s, 0.60H), 2.18 (s, 0.97H), 2.41-2.76 (m, 2H), 3.12-3.28 (m, 0.5H), 3.45-3.79 (m, 2.5H), 3.87-4.41 (m, 3H), 4.90-5.06 (m, 2H), 5.67-5.79 (m, 0.5H), 5.90-6.06 (m, 0.5H), 6.89-7.03 (m, 3H), 7.19-7.31 (m, 2H), 7.50 (dd, J = 8.1, 5.0 Hz, 1H), 7.99 (s, 0.20H), 8.00 (s, 0.30H), 8.07 (s, 0.22H), 8.08 (s, 0.27H), 8.33- 8.39 (m, 1H), 8.48-8.54 (m, 1H), 9.09-9.15 (m, 1H). Enantiomer 1: SFC-Chiral purity = >99.9%, RT = 5.51 min; | 437.2 (M + 1) |
| 3 | 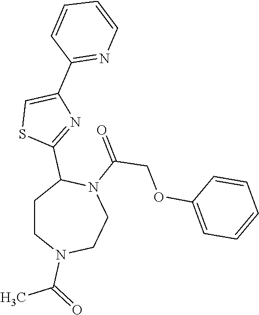 [SMILES: CC(=O)N1CCC(c2nc(-c3ccccn3)cs2)N(C(=O)COc2ccccc2)CC1] | | |
| 4 | 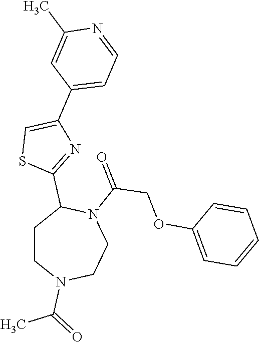 [SMILES: CC(=O)N1CCC(c2nc(-c3ccnc(C)c3)cs2)N(C(=O)COc2ccccc2)CC1] | ¹H NMR (400 MHz, MeOH-d₄) δ 2.10 (s, 0.86H), 2.15 (s, 0.56H), 2.16 (s, 0.65H), 2.20 (s, 0.95H), 2.42-2.79 (m, 5H), 3.13-3.26 (m, 0.5H), 3.49-3.79 (m, 2.5H), 3.90-4.42 (m, 3H), 4.91-5.06 (m, 2H), 5.71-5.81 (m, 0.5H), 5.93-6.06 (m, 0.5H), 6.90-7.04 (m, 3H), 7.21-7.34 (m, 2H), 7.77 (dd, J = 5.4, 1.6 Hz, 1H), 7.86 (d, J = 6.8 Hz, 1H), 8.14-8.25 (m, 1H), 8.43-8.47 (m, 1H). Enantiomer 1: SFC-Chiral purity = >99.9%, RT = 5.06 min; Enantiomer 2: SFC-Chiral purity = >99.9%, RT = 7.95 min | 451.2 (M + 1) |
| 5 | 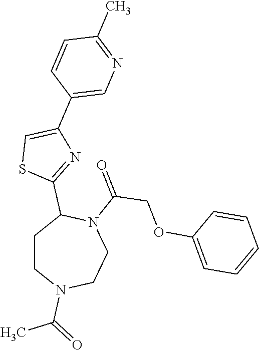 [SMILES: CC(=O)N1CCC(c2nc(-c3ccc(C)nc3)cs2)N(C(=O)COc2ccccc2)CC1] | ¹H NMR (400 MHz, MeOH-d₄) δ 2.09 (s, 0.90H), 2.14 (s, 0.56H), 2.16 (s, 0.66H), 2.19 (s, 0.97H), 2.41-2.76 (m, 5H), 3.13-3.27 (m, 0.5H), 3.49-3.79 (m, 2.5H), 3.89-4.42 (m, 3H), 4.93-5.05 (m, 2H), 5.70-5.79 (m, 0.5H), 5.95-6.05 (m, 0.5H), 6.91-7.04 (m, 3H), 7.21-7.33 (m, 2H), 7.39 (d, J = 8.0 Hz, 1H), 7.92 (s, 0.21H), 7.93 (s, 0.30H), 8.01 (s, 0.18H), 8.01 (s, 0.25H), 8.25 (dd, J = 8.0, 2.0 Hz, 1H), 8.97-9.01 (m, 1H). Enantiomer 1: SFC-Chiral purity = >99.9%, RT = 4.61 min; Enantiomer 2: SFC-Chiral purity = >99.9%, RT = 7.53 min | 451.1 (M + 1) |
| 6 | 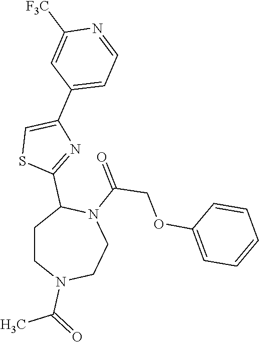 [SMILES: CC(=O)N1CCC(c2nc(-c3ccnc(C(F)(F)F)c3)cs2)N(C(=O)COc2ccccc2)CC1] | | |
| 7 | 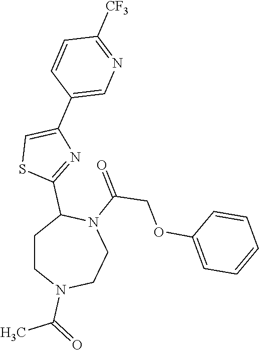 [SMILES: CC(=O)N1CCC(c2nc(-c3ccc(C(F)(F)F)nc3)cs2)N(C(=O)COc2ccccc2)CC1] | ¹H NMR (400 MHz, MeOH-d₄) δ 2.09 (s, 0.85H), 2.14 (s, 0.53H), 2.16 (s, 0.68H), 2.20 (s, 0.97H), 2.43-2.79 (m, 2H), 3.13-3.31 (m, 0.5H), 3.48-3.79 (m, 2.5H), 3.91-4.43 (m, 3H), 4.93-5.06 (m, 2H), 5.73-5.83 (m, 0.5H), 5.96-6.07 (m, 0.5H), 6.89-7.06 (m, 3H), 7.20-7.35 (m, 2H), 7.89 (dd, J = 8.2, 0.8 Hz, 1H), 8.17 (s, 0.22H), 8.18 (s, 0.30H), 8.25 (s, 0.16H), 8.26 (s, 0.25H), 8.51- 8.59 (m, 1H), 9.25-9.32 (m, 1H). | 505.2 (M + 1) |
| 8 | 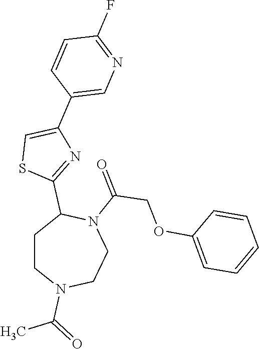 [SMILES: CC(=O)N1CCC(c2nc(-c3ccc(F)nc3)cs2)N(C(=O)COc2ccccc2)CC1] | | |
| 9 | 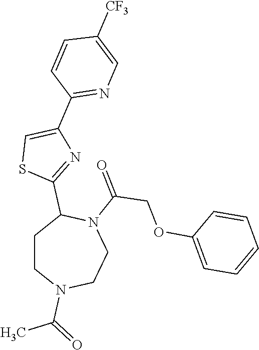 [SMILES: CC(=O)N1CCC(c2nc(-c3ccc(C(F)(F)F)cn3)cs2)N(C(=O)COc2ccccc2)CC1] | | |
| 10 | 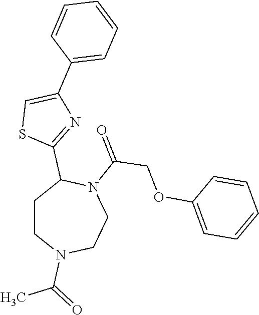 [SMILES: CC(=O)N1CCC(c2nc(-c3ccccc3)cs2)N(C(=O)COc2ccccc2)CC1] | ¹H NMR (400 MHz, MeOH-d₄) δ 2.10 (s, 0.94H), 2.14 (s, 0.56H), 2.15 (s, 0.55H), 2.19 (s, 0.95H), 2.43-2.78 (m, 2H), 3.13-3.30 (m, 0.5H), 3.49-3.77 (m, 2.5H), 3.89-4.42 (m, 3H), 4.93-5.07 (m, 2H), 5.66-5.76 (m, 0.5H), 5.96-6.10 (m, 0.5H), 6.92-7.05 (m, 3H), 7.22-7.38 (m, 3H), 7.39-7.47 (m, 2H), 7.76 (s, 0.21H), 7.77 (s, 0.28H), 7.85 (s, 0.19H), 7.86 (s, 0.25H), 7.91- 7.98 (m, 2H). | 436.2 (M + 1) |
| 11 | 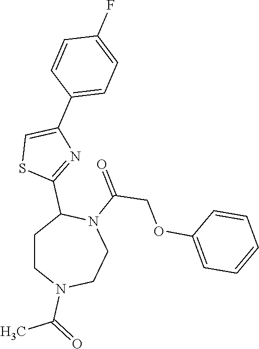 [SMILES: CC(=O)N1CCC(c2nc(-c3ccc(F)cc3)cs2)N(C(=O)COc2ccccc2)CC1] | | |
| 12 | 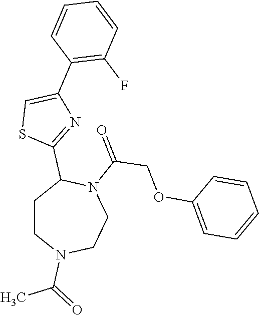 [SMILES: CC(=O)N1CCC(c2nc(-c3ccccc3F)cs2)N(C(=O)COc2ccccc2)CC1] | ¹H NMR (400 MHz, MeOH-d₄) δ 2.09 (s, 0.93H), 2.14 (s, 0.59H), 2.15 (s, 0.64H), 2.19 (s, 0.96H), 2.45-2.78 (m, 2H), 3.12-3.27 (m, 0.5H), 3.49-3.76 (m, 2.5H), 3.96-4.44 (m, 3H), 4.93-5.07 (m, 2H), 5.69-5.79 (m, 0.5H), 5.99-6.10 (m, 0.5H), 6.91-7.06 (m, 3H), 7.18-7.42 (m, 5H), 7.81-7.94 (m, 1H), 8.13-8.22 (m, 1H). Enantiomer 1: SFC-Chiral purity = >99.9%, RT = 3.55 min; Enantiomer 2: SFC-Chiral purity = >99.9%, RT = 6.71 min | 454.2 (M + 1) |
| 13 | 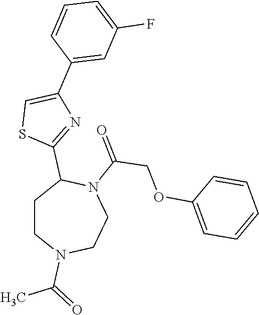 [SMILES: CC(=O)N1CCC(c2nc(-c3cccc(F)c3)cs2)N(C(=O)COc2ccccc2)CC1] | | |
| 14 | 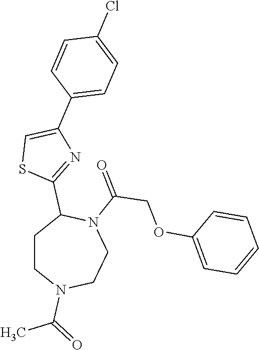 [SMILES: CC(=O)N1CCC(c2nc(-c3ccc(Cl)cc3)cs2)N(C(=O)COc2ccccc2)CC1] | | |
| 15 | 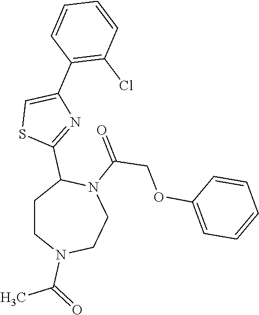 [SMILES: CC(=O)N1CCC(c2nc(-c3ccccc3Cl)cs2)N(C(=O)COc2ccccc2)CC1] | | |
| 16 | 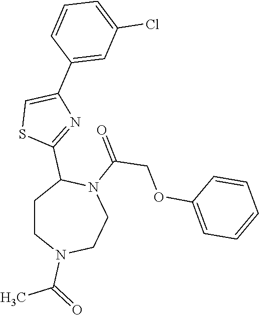 [SMILES: CC(=O)N1CCC(c2nc(-c3cccc(Cl)c3)cs2)N(C(=O)COc2ccccc2)CC1] | | |
| 17 | 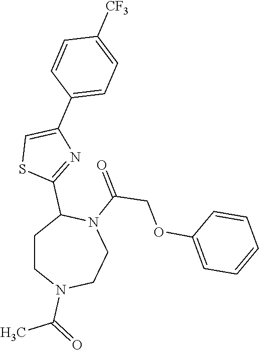 [SMILES: CC(=O)N1CCC(c2nc(-c3ccc(C(F)(F)F)cc3)cs2)N(C(=O)COc2ccccc2)CC1] | ¹H NMR (400 MHz, MeOH-d₄) δ 2.09 (s, 0.84H), 2.14 (s, 0.56H), 2.16 (s, 0.66H), 2.19 (s, 0.90H), 2.45-2.78 (m, 2H), 3.13-3.27 (m, 0.5H), 3.49-3.77 (m, 2.5H), 3.91-4.42 (m, 3H), 4.93-5.06 (m, 2H), 5.70-5.80 (m, 0.5H), 5.97-6.08 (m, 0.5H), 6.91-7.04 (m, 3H), 7.21-7.33 (m, 2H), 7.74 (d, J = 8.4 Hz, 2H), 7.98 (s, 0.21H), 7.99 (s, 0.30H), 8.07 (s, 0.17H), 8.07 (s, 0.26H), 8.15 (d, J = 8.0 Hz, 2H). | 504.2 (M + 1) |
| 18 | 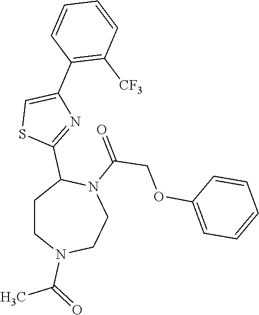 [SMILES: CC(=O)N1CCC(c2nc(-c3ccccc3C(F)(F)F)cs2)N(C(=O)COc2ccccc2)CC1] | | |
| 19 | 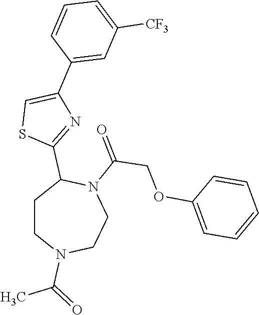 [SMILES: CC(=O)N1CCC(c2nc(-c3cccc(C(F)(F)F)c3)cs2)N(C(=O)COc2ccccc2)CC1] | | |
| 20 | 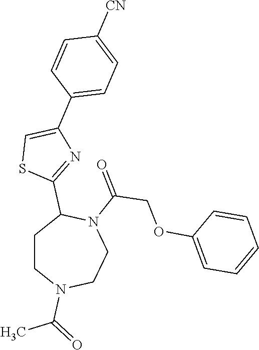 [SMILES: CC(=O)N1CCC(c2nc(-c3ccc(C#N)cc3)cs2)N(C(=O)COc2ccccc2)CC1] | | |
| 21 | 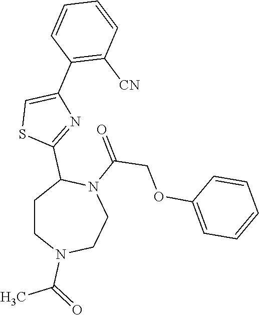 [SMILES: CC(=O)N1CCC(c2nc(-c3ccccc3C#N)cs2)N(C(=O)COc2ccccc2)CC1] | | |
| 22 | 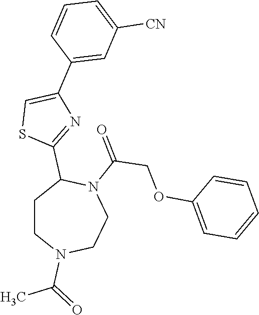 [SMILES: CC(=O)N1CCC(c2nc(-c3cccc(C#N)c3)cs2)N(C(=O)COc2ccccc2)CC1] | | |
| 23 | 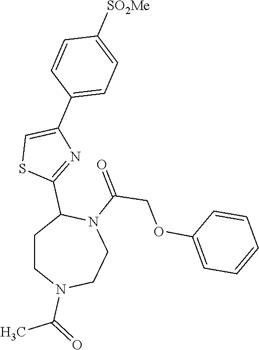 [SMILES: CC(=O)N1CCC(c2nc(-c3ccc(S(C)(=O)=O)cc3)cs2)N(C(=O)COc2ccccc2)CC1] | ¹H NMR (400 MHz, MeOH-d₄) δ 2.09 (s, 0.82H), 2.15 (s, 0.54H), 2.16 (s, 0.60H), 2.19 (s, 0.91H), 2.48-2.79 (m, 2H), 3.17 (s, 3H), 3.20-3.31 (m, 0.5H), 3.47-3.81 (m, 2.5H), 3.89-4.43 (m, 3H), 4.92-5.07 (m, 2H), 5.72-5.81 (m, 0.5H), 5.96-6.08 (m, 0.5H), 6.89-7.06 (m, 3H), 7.20-7.35 (m, 2H), 7.98-8.06 (m, 2H), 8.06 (s, 0.20H), 8.08 (s, 0.29H), 8.15 (s, 0.15H), 8.15 (s, 0.25H), 8.18- 8.26 (m, 2H). | 513.9 (M + 1) |
| 24 | 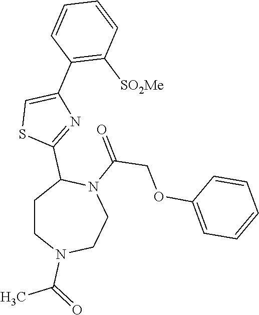 [SMILES: CC(=O)N1CCC(c2nc(-c3ccccc3S(C)(=O)=O)cs2)N(C(=O)COc2ccccc2)CC1] | | |
| 25 | 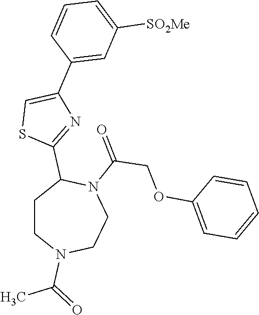 [SMILES: CC(=O)N1CCC(c2nc(-c3cccc(S(C)(=O)=O)c3)cs2)N(C(=O)COc2ccccc2)CC1] | | |
| 26 | 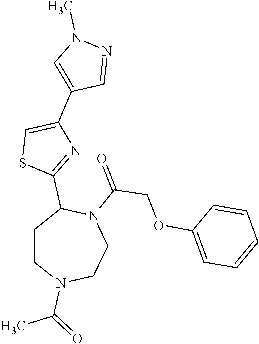 [SMILES: CC(=O)N1CCC(c2nc(-c3cnn(C)c3)cs2)N(C(=O)COc2ccccc2)CC1] | ¹H NMR (400 MHz, MeOH-d₄) δ 2.09 (s, 0.90H), 2.14 (s, 0.59H), 2.15 (s, 0.61H), 2.18 (s, 0.91H), 2.38-2.76 (m, 2H), 3.15-3.29 (m, 0.5H), 3.45-3.77 (m, 2.5H), 3.94 (s, 3H), 3.94-4.41 (m, 3H), 4.91-5.06 (m, 2H), 5.62-5.72 (m, 0.5H), 5.91-6.04 (m, 0.5H), 6.89-7.05 (m, 3H), 7.21-7.35 (m, 2H), 7.45 (s, 0.20H), 7.46 (s, 0.29H), 7.53 (s, 0.18H), 7.54 (s, 0.27H), 7.84-7.90 (m, 1H), 7.97- 8.03 (m, 1H). | 440.2 (M + 1) |
| 27 | 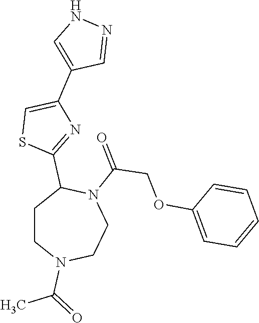 [SMILES: CC(=O)N1CCC(c2nc(-c3cn[nH]c3)cs2)N(C(=O)COc2ccccc2)CC1] | ¹H NMR (400 MHz, MeOH-d₄) δ 2.09 (s, 0.82H), 2.14 (s, 0.51H), 2.15 (s, 0.73H), 2.19 (s, 1.02H), 2.41-2.78 (m, 2H), 3.13-3.29 (m, 0.5H), 3.47-3.76 (m, 2.5H), 3.92-4.41 (m, 3H), 4.93-5.04 (m, 2H), 5.68-5.77 (m, 0.5H), 5.91-5.98 (m, 0.5H), 6.49-6.53 (m, 1H), 6.90-7.04 (m, 3H), 7.22-7.33 (m, 2H), 7.46 (s, 0.22H), 7.47 (s, 0.31H), 7.54 (s, 0.19H), 7.55 (s, 0.23H), 7.71- 7.76 (m, 1H), 8.28-8.36 (m, 1H). | 426.2 |
| 28 | 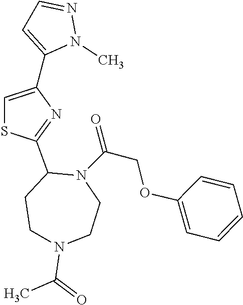 [SMILES: CC(=O)N1CCC(c2nc(-c3ccnn3C)cs2)N(C(=O)COc2ccccc2)CC1] | | |
| 29 | 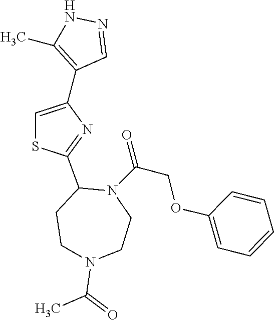 [SMILES: CC(=O)N1CCC(c2nc(-c3cn[nH]c3C)cs2)N(C(=O)COc2ccccc2)CC1] | | |
| 30 | 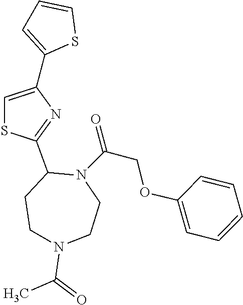 [SMILES: CC(=O)N1CCC(c2nc(-c3cccs3)cs2)N(C(=O)COc2ccccc2)CC1] | | |
| 31 | 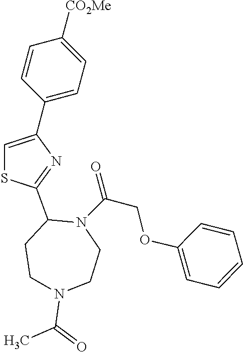 [SMILES: COC(=O)c1ccc(-c2csc(C3CCN(C(C)=O)CCN3C(=O)COc3ccccc3)n2)cc1] | | |
| 32 | 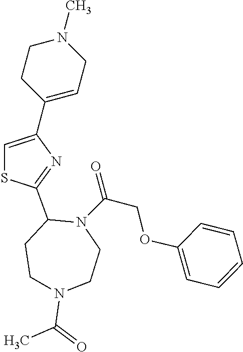 [SMILES: CC(=O)N1CCC(c2nc(C3=CCN(C)CC3)cs2)N(C(=O)COc2ccccc2)CC1] | | |
| 33 | 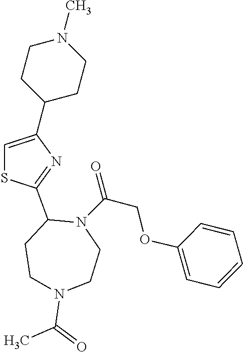 [SMILES: CC(=O)N1CCC(c2nc(C3CCN(C)CC3)cs2)N(C(=O)COc2ccccc2)CC1] | | |
| 34 | 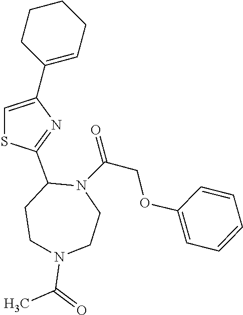 [SMILES: CC(=O)N1CCC(c2nc(C3=CCCCC3)cs2)N(C(=O)COc2ccccc2)CC1] | | |
| 35 | 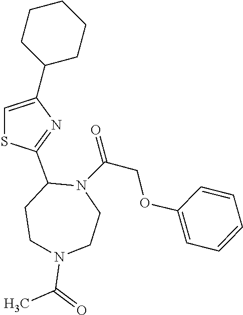 [SMILES: CC(=O)N1CCC(c2nc(C3CCCCC3)cs2)N(C(=O)COc2ccccc2)CC1] | | |
| 36 | 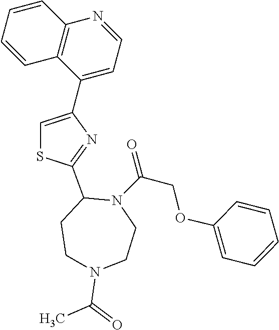 [SMILES: CC(=O)N1CCC(c2nc(-c3ccnc4ccccc34)cs2)N(C(=O)COc2ccccc2)CC1] | | |
| 37 | 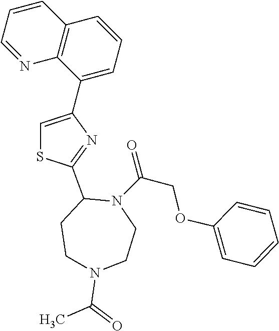 [SMILES: CC(=O)N1CCC(c2nc(-c3cccc4cccnc34)cs2)N(C(=O)COc2ccccc2)CC1] | | |
| 38 | 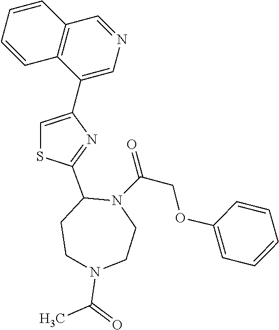 [SMILES: CC(=O)N1CCC(c2nc(-c3cncc4ccccc34)cs2)N(C(=O)COc2ccccc2)CC1] | | |
| 39 | 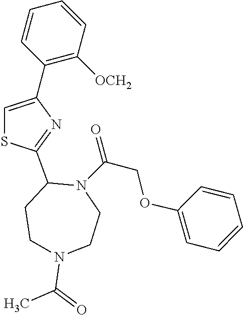 | | |
| 40 | 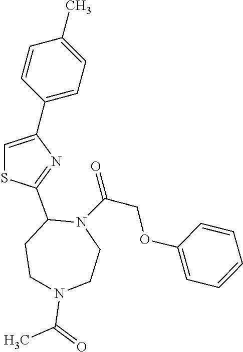 [SMILES: CC(=O)N1CCC(c2nc(-c3ccc(C)cc3)cs2)N(C(=O)COc2ccccc2)CC1] | ¹H NMR (400 MHz, MeOH-d₄) δ 2.10 (s, 0.95H), 2.14 (s, 0.62H), 2.15 (s, 0.56H), 2.19 (s, 0.91H), 2.39 (s, 3H), 2.43-2.77 (m, 2H), 3.14-3.26 (m, 0.5H), 3.48-3.77 (m, 2.5H), 3.88-4.43 (m, 3H), 4.93-5.07 (m, 2H), 5.64-5.76 (m, 0.5H), 5.96-6.09 (m, 0.5H), 6.91-7.05 (m, 3H), 7.22-7.33 (m, 4H), 7.68 (s, 0.18H), 7.70 (s, 0.27H), 7.77 (s, 0.23H), 7.78 (s, 0.26H), 7.79-7.86 (m, 2H). | 450.3 (M + 1) |
| 41 | 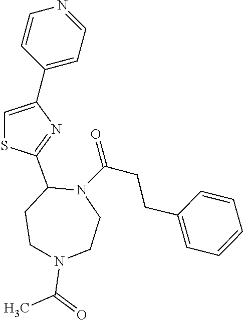 [SMILES: CC(=O)N1CCC(c2nc(-c3ccncc3)cs2)N(C(=O)CCc2ccccc2)CC1] | ¹H NMR (400 MHz, MeOH-d₄) δ 2.09 (s, 0.79H), 2.10 (s, 0.69H), 2.15 (s, 0.46H), 2.17 (s, 0.99H), 2.32-2.64 (m, 2H), 2.74-2.93 (m, 2H), 2.95-3.21 (m, 4H), 3.39-3.59 (m, 1H), 3.69-4.39 (m, 3H), 5.58 (q, J = 6.8 Hz, 0.5H), 5.95-6.06 (m, 0.5H), 7.13-7.21 (m, 1H), 7.21-7.29 (m, 4H), 7.93-8.01 (m, 2H), 8.18 (s, 0.26H), 8.19 (s, 0.31H), 8.22 (s, 0.15H), 8.23 (s, 0.21H), 8.55- 8.63 (m, 2H). | 435.2 (M + 1) |
| 42 | 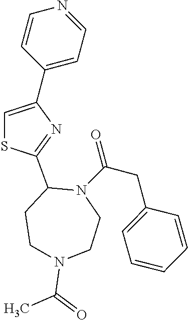 [SMILES: CC(=O)N1CCC(c2nc(-c3ccncc3)cs2)N(C(=O)Cc2ccccc2)CC1] | ¹H NMR (400 MHz, MeOH-d₄) δ 2.07 (s, 0.82H), 2.08 (s, 0.54H), 2.10 (s, 0.74H), 2.18 (s, 0.93H), 2.21-2.68 (m, 2H), 3.03-3.20 (m, 0.5H), 3.35-3.68 (m, 2.5H), 3.76-4.40 (m, 5H), 5.69 (t, J = 6.4 Hz, 0.5H), 5.99-6.09 (m, 0.5H), 7.24-7.31 (m, 1H), 7.31- 7.37 (m, 4H), 7.93-8.00 (m, 2H), 8.18 (s, 0.25H), 8.18 (s, 0.28H), 8.22 (s, 0.23H), 8.23 (s, 0.17H), 8.55-8.63 (m, 2H). | 421.2 (M + 1) |
| 43 | 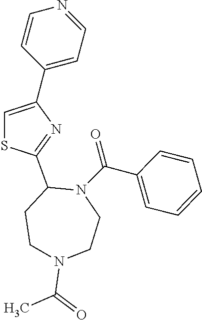 [SMILES: CC(=O)N1CCC(c2nc(-c3ccncc3)cs2)N(C(=O)c2ccccc2)CC1] | ¹H NMR (400 MHz, MeOH-d₄) δ 2.06 (s, 0.64H), 2.20 (s, 2.36H), 2.38-2.87 (m, 2H), 3.46-4.10 (m, 5H), 4.15-4.61 (m, 1H), 5.30-5.42 (m, 0.5H), 6.04-6.19 (m, 0.5H), 7.50 (s, 5H), 8.01 (s, 2H), 8.23-8.32 (m, 1H), 8.57- 8.64 (m, 2H). Enantiomer 1: SFC-Chiral purity = 99.1%, RT = 7.90 min; Enantiomer 2: SFC-Chiral purity = 98.9%, RT = 11.95 min | 407.2 (M+1) |
| 44 | 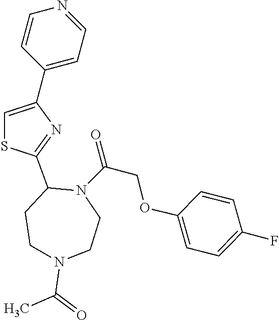 [SMILES: CC(=O)N1CCC(c2nc(-c3ccncc3)cs2)N(C(=O)COc2ccc(F)cc2)CC1] | ¹H NMR (400 MHz, MeOH-d₄) δ 2.10 (s, 0.81H), 2.16 (s, 0.45H), 2.17 (s, 0.64H), 2.19 (s, 0.96H), 2.43-2.80 (m, 2H), 3.13-3.29 (m, 0.5H), 3.50-3.78 (m, 2.5H), 3.92-4.44 (m, 3H), 4.91-5.04 (m, 2H), 5.68-5.78 (m, 0.5H), 5.94-6.06 (m, 0.5H), 6.83-7.07 (m, 4H), 7.93-8.01 (m, 2H), 8.19 (s, 0.21H), 8.20 (s, 0.30H), 8.27 (s, 0.19H), 8.27 (s, 0.21H), 8.52- 8.64 (m, 2H). | 455.2 (M + 1) |
| 45 | 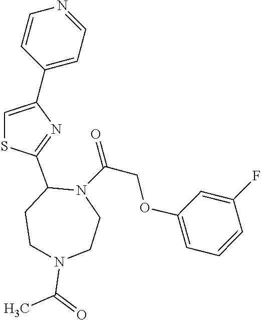 [SMILES: CC(=O)N1CCC(c2nc(-c3ccncc3)cs2)N(C(=O)COc2cccc(F)c2)CC1] | | |
| 46 | 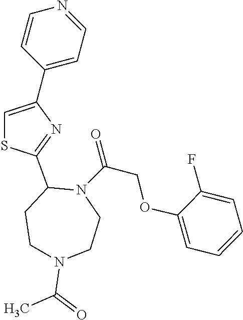 [SMILES: CC(=O)N1CCC(c2nc(-c3ccncc3)cs2)N(C(=O)COc2ccccc2F)CC1] | | |
| 47 | 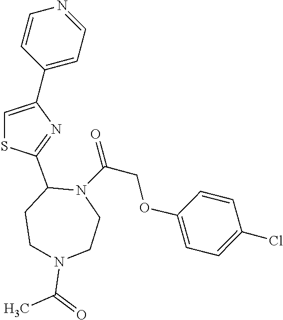 [SMILES: CC(=O)N1CCC(c2nc(-c3ccncc3)cs2)N(C(=O)COc2ccc(Cl)cc2)CC1] | | |
| 48 | 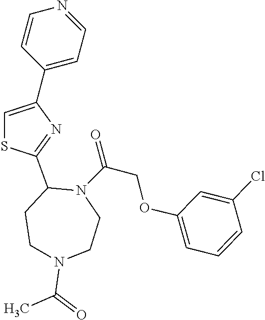 [SMILES: CC(=O)N1CCC(c2nc(-c3ccncc3)cs2)N(C(=O)COc2cccc(Cl)c2)CC1] | | |
| 49 | 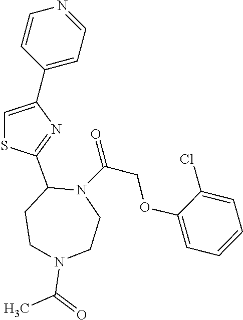 [SMILES: CC(=O)N1CCC(c2nc(-c3ccncc3)cs2)N(C(=O)COc2ccccc2Cl)CC1] | | |
| 50 | 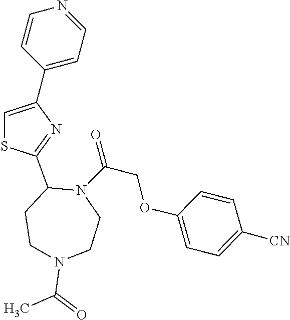 [SMILES: CC(=O)N1CCC(c2nc(-c3ccncc3)cs2)N(C(=O)COc2ccc(C#N)cc2)CC1] | ¹H NMR (400 MHz, MeOH-d₄) δ 2.10 (s, 0.77H), 2.17 (s, 0.47H), 2.18 (s, 0.63H), 2.20 (s, 1.03H), 2.42-2.83 (m, 2H), 3.14-3.28 (m, 0.5H), 3.47-3.83 (m, 2.5H), 3.92-4.44 (m, 3H), 5.03-5.21 (m, 2H), 5.65-5.74 (m, 0.5H), 5.92-6.04 (m, 0.5H), 7.03-7.19 (m, 2H), 7.58-7.71 (m, 2H), 7.94-8.02 (m, 2H), 8.19 (s, 0.20H), 8.21 (s, 0.33H), 8.28 (s, 0.16H), 8.28 (s, 0.19H), 8.58- 8.65 (m, 2H). | 462.2 (M + 1) |
| 51 | 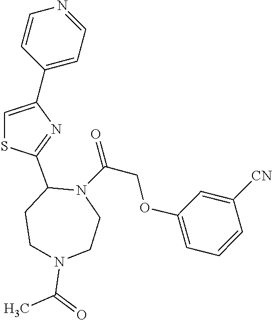 [SMILES: CC(=O)N1CCC(c2nc(-c3ccncc3)cs2)N(C(=O)COc2cccc(C#N)c2)CC1] | | |
| 52 | 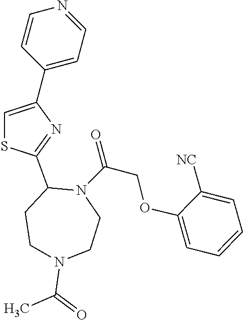 [SMILES: CC(=O)N1CCC(c2nc(-c3ccncc3)cs2)N(C(=O)COc2ccccc2C#N)CC1] | | |
| 53 | 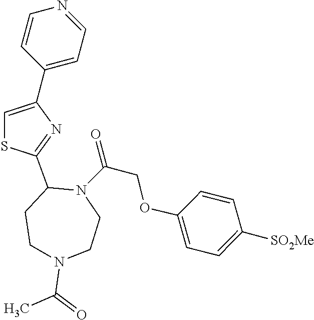 [SMILES: CC(=O)N1CCC(c2nc(-c3ccncc3)cs2)N(C(=O)COc2ccc(S(C)(=O)=O)cc2)CC1] | | |
| 54 | 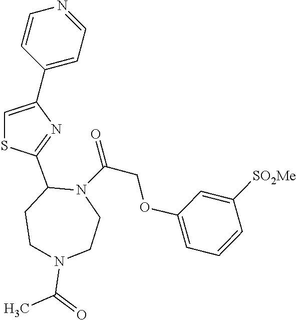 [SMILES: CC(=O)N1CCC(c2nc(-c3ccncc3)cs2)N(C(=O)COc2cccc(S(C)(=O)=O)c2)CC1] | | |
| 55 | 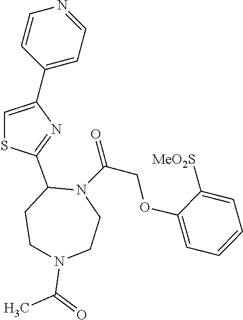 [SMILES: CC(=O)N1CCC(c2nc(-c3ccncc3)cs2)N(C(=O)COc2ccccc2S(C)(=O)=O)CC1] | | |
| 56 | 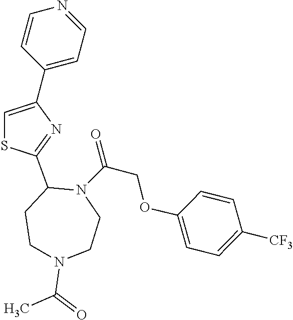 [SMILES: CC(=O)N1CCC(c2nc(-c3ccncc3)cs2)N(C(=O)COc2ccc(C(F)(F)F)cc2)CC1] | ¹H NMR (400 MHz, MeOH-d₄) δ 2.10 (s, 0.77H), 2.17 (s, 0.48H), 2.18 (s, 0.63H), 2.20 (s, 0.99H), 2.42-2.84 (m, 2H), 3.14-3.30 (m, 0.5H), 3.48-3.78 (m, 2.5H), 3.94-4.44 (m, 3H), 4.98-5.17 (m, 2H), 5.69-5.77 (m, 0.5H), 5.94-6.04 (m, 0.5H), 7.03-7.19 (m, 2H), 7.50-7.63 (m, 2H), 7.94-8.01 (m, 2H), 8.19 (s, 0.21H), 8.21 (s, 0.33H), 8.28 (s, 0.15H), 8.28 (s, 0.23H), 8.56- 8.62 (m, 2H). | 505.2 (M + 1) |
| 57 | 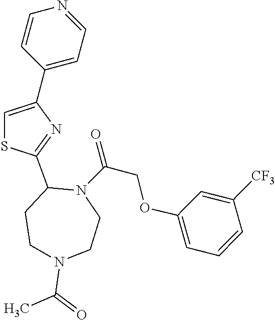 [SMILES: CC(=O)N1CCC(c2nc(-c3ccncc3)cs2)N(C(=O)COc2cccc(C(F)(F)F)c2)CC1] | | |
| 58 | 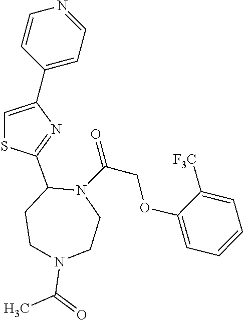 [SMILES: CC(=O)N1CCC(c2nc(-c3ccncc3)cs2)N(C(=O)COc2ccccc2C(F)(F)F)CC1] | | |
| 59 | 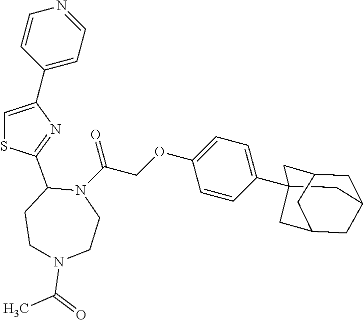 [SMILES: CC(=O)N1CCC(c2nc(-c3ccncc3)cs2)N(C(=O)COc2ccc(C34CC5CC(CC(C5)C3)C4)cc2)CC1] | | |
| 60 | 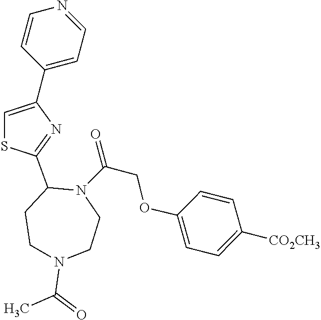 [SMILES: COC(=O)c1ccc(OCC(=O)N2CCN(C(C)=O)CCC2c2nc(-c3ccncc3)cs2)cc1] | | |
| 61 | 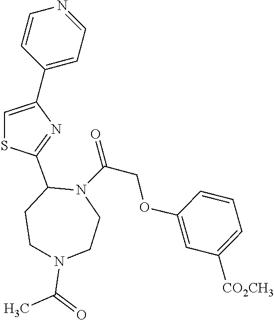 [SMILES: COC(=O)c1cccc(OCC(=O)N2CCN(C(C)=O)CCC2c2nc(-c3ccncc3)cs2)c1] | | |
| 62 | 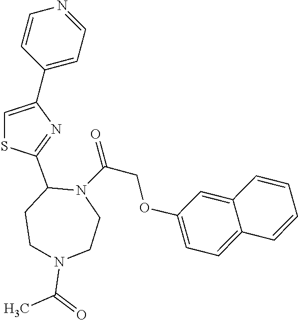 [SMILES: CC(=O)N1CCC(c2nc(-c3ccncc3)cs2)N(C(=O)COc2ccc3ccccc3c2)CC1] | | |
| 63 | 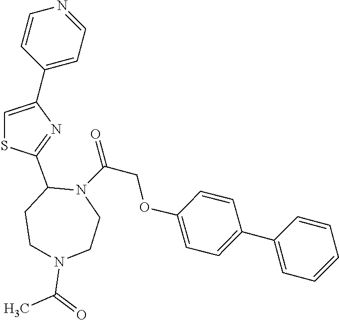 [SMILES: CC(=O)N1CCC(c2nc(-c3ccncc3)cs2)N(C(=O)COc2ccc(-c3ccccc3)cc2)CC1] | | |
| 64 | 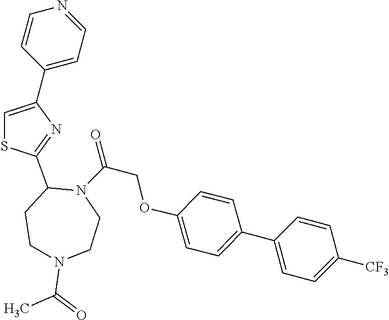 [SMILES: CC(=O)N1CCC(c2nc(-c3ccncc3)cs2)N(C(=O)COc2ccc(-c3ccc(C(F)(F)F)cc3)cc2)CC1] | | |
| 65 | 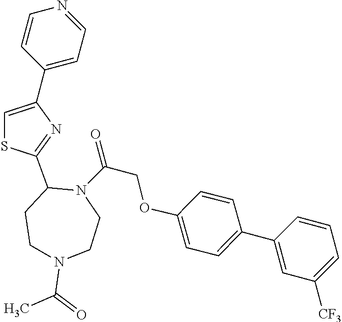 [SMILES: CC(=O)N1CCC(c2nc(-c3ccncc3)cs2)N(C(=O)COc2ccc(-c3cccc(C(F)(F)F)c3)cc2)CC1] | | |
| 66 | 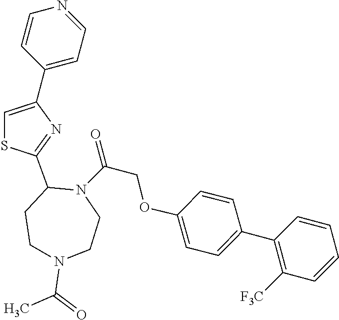 [SMILES: CC(=O)N1CCC(c2nc(-c3ccncc3)cs2)N(C(=O)COc2ccc(-c3ccccc3C(F)(F)F)cc2)CC1] | | |
| 67 | 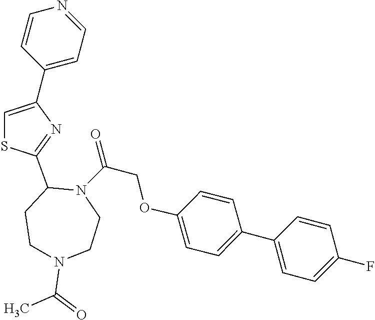 [SMILES: CC(=O)N1CCC(c2nc(-c3ccncc3)cs2)N(C(=O)COc2ccc(-c3ccc(F)cc3)cc2)CC1] | | |
| 68 | 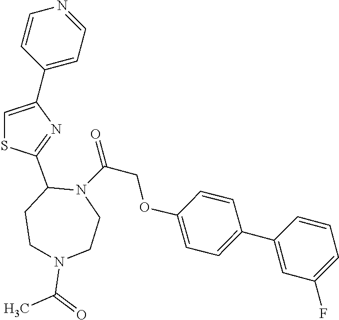 [SMILES: CC(=O)N1CCC(c2nc(-c3ccncc3)cs2)N(C(=O)COc2ccc(-c3cccc(F)c3)cc2)CC1] | | |
| 69 | 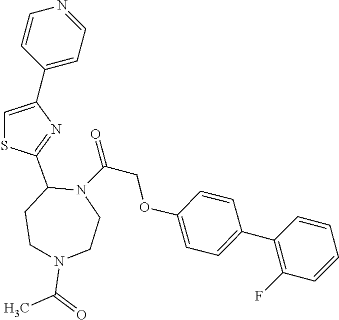 [SMILES: CC(=O)N1CCC(c2nc(-c3ccncc3)cs2)N(C(=O)COc2ccc(-c3ccccc3F)cc2)CC1] | | |
| 70 | 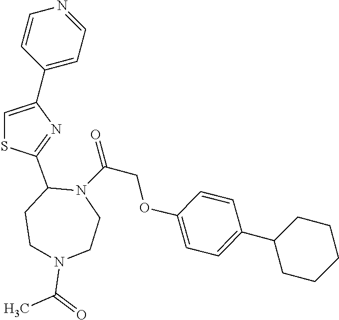 [SMILES: CC(=O)N1CCC(c2nc(-c3ccncc3)cs2)N(C(=O)COc2ccc(C3CCCCC3)cc2)CC1] | | |
| 71 | 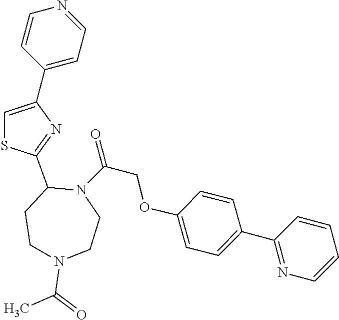 [SMILES: CC(=O)N1CCC(c2nc(-c3ccncc3)cs2)N(C(=O)COc2ccc(-c3ccccn3)cc2)CC1] | | |
| 72 | 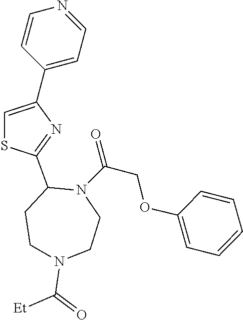 [SMILES: CCC(=O)N1CCC(c2nc(-c3ccncc3)cs2)N(C(=O)COc2ccccc2)CC1] | ¹H NMR (400 MHz, MeOH-d₄) δ 1.05-1.18 (m, 3H), 2.34-2.78 (m, 4H), 3.13-3.27 (m, 0.5H), 3.50-3.77 (m, 3.5H), 3.92-4.44 (m, 3H), 4.93-5.05 (m, 2H), 5.72-5.81 (m, 0.5H), 5.94-6.06 (m, 0.5H), 6.89-7.05 (m, 2H), 7.21-7.33 (m, 2H), 7.94-8.00 (m, 2H), 8.18 (s, 0.21H), 8.19 (s, 0.30H), 8.26 (s, 0.16H), 8.27 (s, 0.24H), 8.56-8.62 (m, 2H). | 451.2 (M + 1) |
| 73 | 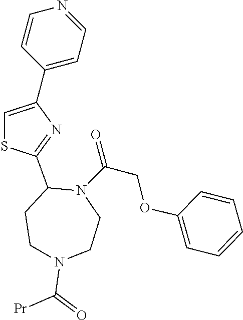 | | |
| 74 | 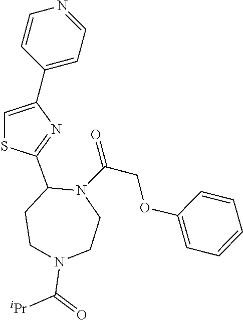 [SMILES: CC(C)C(=O)N1CCC(c2nc(-c3ccncc3)cs2)N(C(=O)COc2ccccc2)CC1] | ¹H NMR (400 MHz, MeOH-d₄) δ 1.05-1.17 (m, 6H), 2.44-2.81 (m, 2H), 2.89-3.08 (m, 1H), 3.13-3.28 (m, 0.5H), 3.51-3.85 (m, 2.5H), 3.89-4.44 (m, 3H), 4.91-5.05 (m, 2H), 5.71-5.81 (m, 0.5H), 5.95-6.05 (m, 0.5H), 6.88-7.05 (m, 3H), 7.20-7.34 (m, 2H), 7.95-8.01 (m, 2H), 8.18 (s, 0.23H), 8.20 (s, 0.33H), 8.27 (s, 0.16H), 8.27 (s, 0.27H), 8.55- 8.63 (m, 2H). | 463.2 (M + 1) |
| 75 | 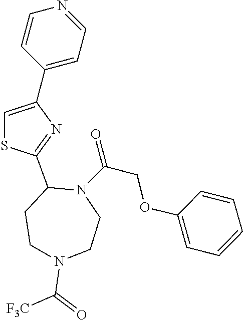 [SMILES: O=C(COc1ccccc1)N1CCN(C(=O)C(F)(F)F)CCC1c1nc(-c2ccncc2)cs1] | | |
| 76 | 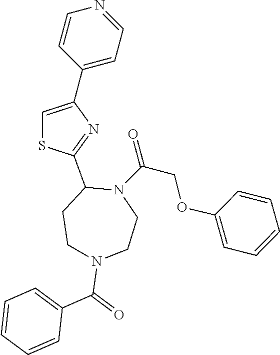 [SMILES: O=C(c1ccccc1)N1CCC(c2nc(-c3ccncc3)cs2)N(C(=O)COc2ccccc2)CC1] | | |
| 77 | 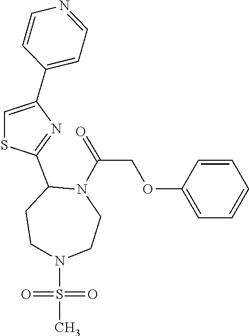 [SMILES: CS(=O)(=O)N1CCC(c2nc(-c3ccncc3)cs2)N(C(=O)COc2ccccc2)CC1] | ¹H NMR (400 MHz, MeOH-d₄) δ 2.45-2.56 (m, 0.5H), 2.61-2.78 (m, 1H), 2.88 (s, 1.35H), 2.93 (s, 1.76H), 3.10-3.24 (m, 1.5H), 3.37-3.53 (m, 1H), 3.74-4.04 (m, 3H), 4.11-4.50 (m, 1H), 4.93-5.07 (m, 2H), 5.66-5.73 (m, 0.5H), 5.90-6.00 (m, 0.5H), 6.87-7.05 (m, 3H), 7.18-7.33 (m, 2H), 7.95-8.02 (m, 2H), 8.18 (s, 0.56H), 8.25 (s, 0.40H), 8.55- 8.63 (m, 2H). | 473.2 (M + 1) |
| 78 | 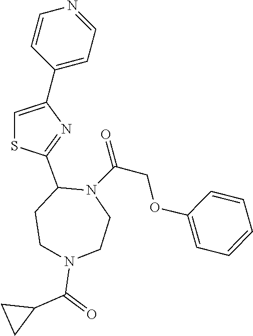 [SMILES: O=C(C1CC1)N1CCC(c2nc(-c3ccncc3)cs2)N(C(=O)COc2ccccc2)CC1] | | |
| 79 | 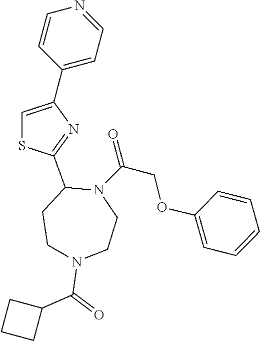 [SMILES: O=C(C1CCC1)N1CCC(c2nc(-c3ccncc3)cs2)N(C(=O)COc2ccccc2)CC1] | | |
| 80 | 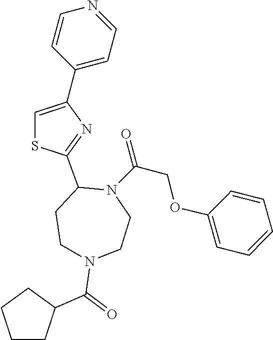 [SMILES: O=C(C1CCCC1)N1CCC(c2nc(-c3ccncc3)cs2)N(C(=O)COc2ccccc2)CC1] | | |
| 81 | 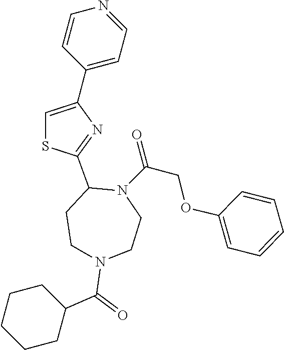 [SMILES: O=C(C1CCCCC1)N1CCC(c2nc(-c3ccncc3)cs2)N(C(=O)COc2ccccc2)CC1] | | |
| 82 | 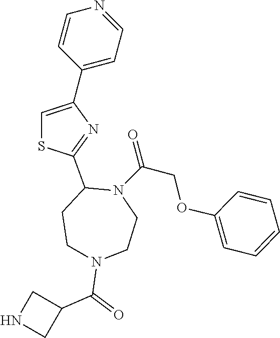 [SMILES: O=C(C1CNC1)N1CCC(c2nc(-c3ccncc3)cs2)N(C(=O)COc2ccccc2)CC1] | | |
| 83 | 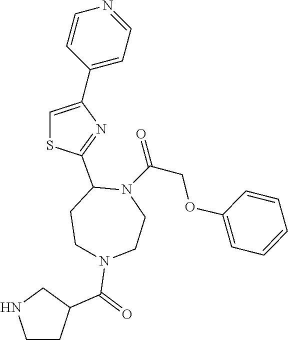 [SMILES: O=C(C1CCNC1)N1CCC(c2nc(-c3ccncc3)cs2)N(C(=O)COc2ccccc2)CC1] | | |
| 84 | 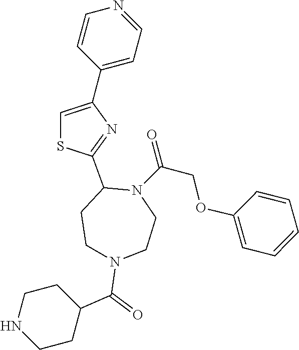 [SMILES: O=C(C1CCNCC1)N1CCC(c2nc(-c3ccncc3)cs2)N(C(=O)COc2ccccc2)CC1] | | |
| 85 | 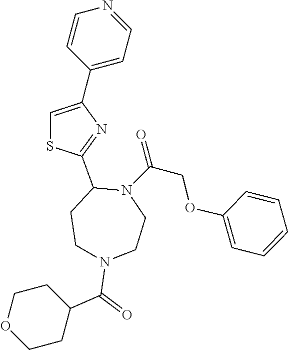 [SMILES: O=C(C1CCOCC1)N1CCC(c2nc(-c3ccncc3)cs2)N(C(=O)COc2ccccc2)CC1] | | |
| 86 | 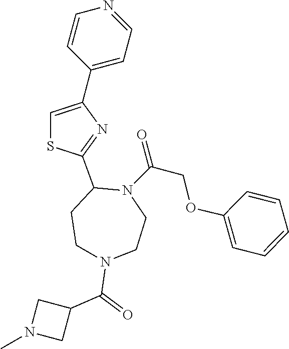 [SMILES: CN1CC(C(=O)N2CCC(c3nc(-c4ccncc4)cs3)N(C(=O)COc3ccccc3)CC2)C1] | | |
| 87 | 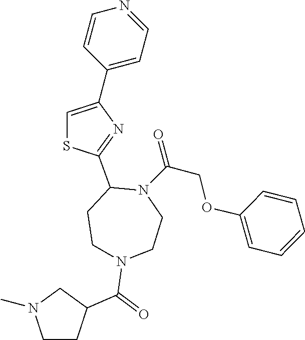 [SMILES: CN1CCC(C(=O)N2CCC(c3nc(-c4ccncc4)cs3)N(C(=O)COc3ccccc3)CC2)C1] | | |
| 88 | 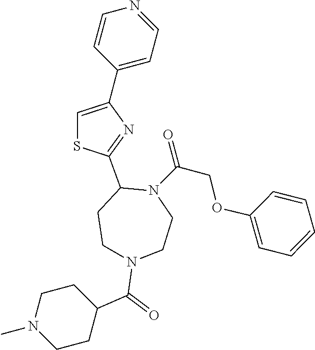 [SMILES: CN1CCC(C(=O)N2CCC(c3nc(-c4ccncc4)cs3)N(C(=O)COc3ccccc3)CC2)CC1] | | |
| 89 | 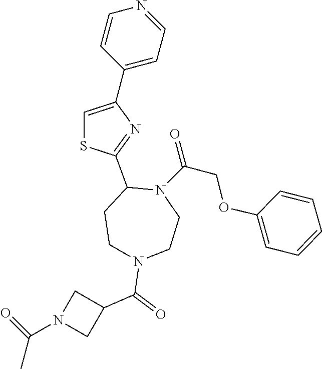 [SMILES: CC(=O)N1CC(C(=O)N2CCC(c3nc(-c4ccncc4)cs3)N(C(=O)COc3ccccc3)CC2)C1] | | |
| 90 | 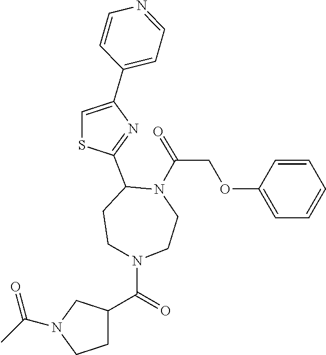 [SMILES: CC(=O)N1CCC(C(=O)N2CCC(c3nc(-c4ccncc4)cs3)N(C(=O)COc3ccccc3)CC2)C1] | | |
| 91 | 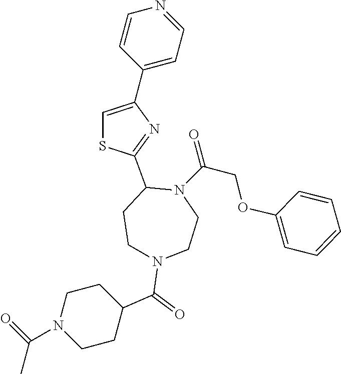 [SMILES: CC(=O)N1CCC(C(=O)N2CCC(c3nc(-c4ccncc4)cs3)N(C(=O)COc3ccccc3)CC2)CC1] | | |
| 92 | 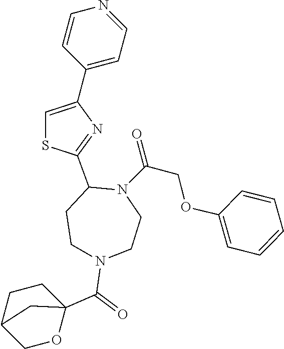 [SMILES: O=C(COc1ccccc1)N1CCN(C(=O)C23CCC(CO2)C3)CCC1c1nc(-c2ccncc2)cs1] | | |
| 93 | 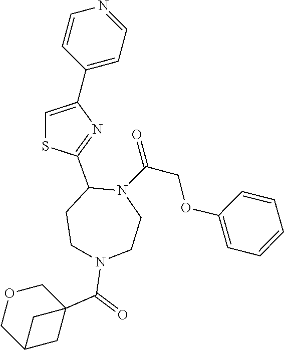 [SMILES: O=C(COc1ccccc1)N1CCN(C(=O)C23COCC(C2)C3)CCC1c1nc(-c2ccncc2)cs1] | | |
| 94 | 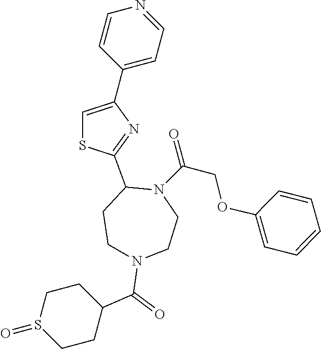 [SMILES: O=C(C1CCS(=O)CC1)N1CCC(c2nc(-c3ccncc3)cs2)N(C(=O)COc2ccccc2)CC1] | | |
| 95 | 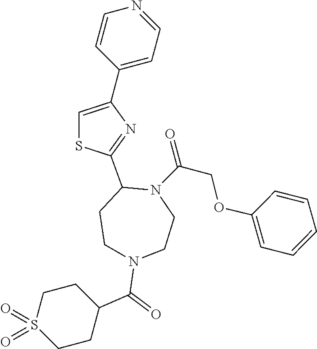 [SMILES: O=C(C1CCS(=O)(=O)CC1)N1CCC(c2nc(-c3ccncc3)cs2)N(C(=O)COc2ccccc2)CC1] | | |
| 96 | 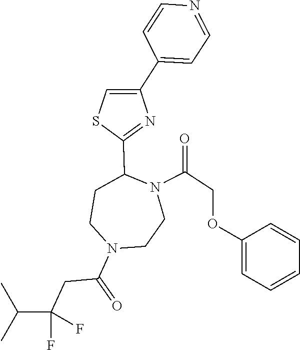 [SMILES: CC(C)C(F)(F)CC(=O)N1CCC(c2nc(-c3ccncc3)cs2)N(C(=O)COc2ccccc2)CC1] | | |
| 97 | 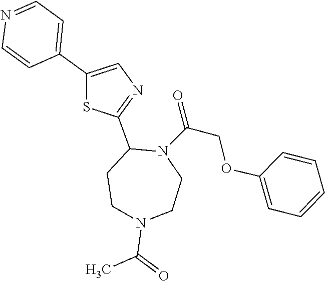 [SMILES: CC(=O)N1CCC(c2ncc(-c3ccncc3)s2)N(C(=O)COc2ccccc2)CC1] | | |
| 98 | 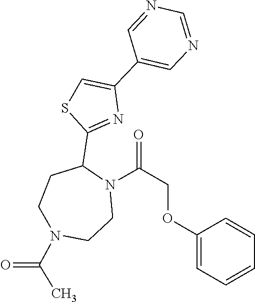 [SMILES: CC(=O)N1CCC(c2nc(-c3cncnc3)cs2)N(C(=O)COc2ccccc2)CC1] | ¹H NMR (400 MHz, MeOH-d₄) δ 2.10 (s, 0.81H), 2.15 (s, 0.52H), 2.16 (s, 0.69H), 2.20 (s, 0.94H), 2.42-2.80 (m, 2H), 3.13-3.29 (m, 0.5H), 3.47-3.82 (m, 2.5H), 3.91-4.43 (m, 3H), 4.93-5.06 (m, 2H), 5.73-5.82 (m, 0.5H), 5.95-6.04 (m, 0.5H), 6.89-7.04 (m, 3H), 7.22-7.34 (m, 2H), 8.16 (s, 0.21H), 8.17 (s, 0.29H), 8.24 (s, 0.17H), 8.24 (s, 0.23H), 9.11- 9.16 (m, 1H), 9.30-9.36 (m, 2H). | 438.0 (M + 1) |
| 99 | 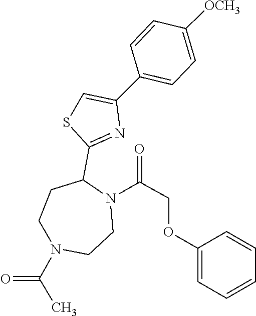 [SMILES: COc1ccc(-c2csc(C3CCN(C(C)=O)CCN3C(=O)COc3ccccc3)n2)cc1] | ¹H NMR (400 MHz, MeOH-d₄) δ 2.09 (s, 0.95H), 2.14 (s, 0.55H), 2.15 (s, 0.57H), 2.19 (s, 0.92H), 2.42-2.78 (m, 2H), 3.13-3.29 (m, 0.5H), 3.47-3.77 (m, 2.5H), 3.85 (s, 3H), 3.97-4.41 (m, 3H), 4.93-5.07 (m, 2H), 5.64-5.74 (m, 0.5H), 5.96-6.07 (m, 0.5H), 6.92-7.04 (m, 5H), 7.22-7.33 (m, 2H), 7.59 (s, 0.20H), 7.61 (s, 0.27H), 7.68 (s, 0.18H), 7.69 (s, 0.27H), 7.83-7.91 (m, 2H). | 466.2 (M + 1) |
| 100 | 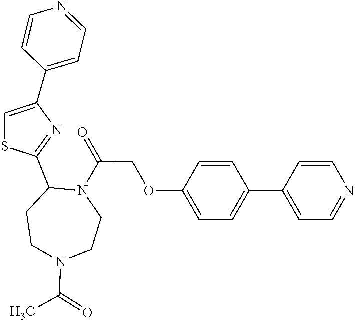 [SMILES: CC(=O)N1CCC(c2nc(-c3ccncc3)cs2)N(C(=O)COc2ccc(-c3ccncc3)cc2)CC1] | ¹H NMR (400 MHz, MeOH-d₄) δ 2.09 (s, 0.78H), 2.15 (s, 0.56H), 2.18 (s, 0.64H), 2.21 (s, 0.92H), 2.42-2.83 (m, 2H), 3.13-3.29 (m, 0.5H), 3.49-3.80 (m, 2.5H), 3.90-4.47 (m, 3H), 5.02-5.15 (m, 2H), 5.73-5.82 (m, 0.5H), 5.95-6.06 (m, 0.5H), 7.03-7.19 (m, 2H), 7.61-7.76 (m, 4H), 7.93-8.00 (m, 2H), 8.18 (s, 0.21H), 8.20 (s, 0.30H), 8.28 (s, 0.16H), 8.28 (s, 0.24H), 8.50- 8.60 (m, 4H). | 514.0 (M + 1) |
| 101 | 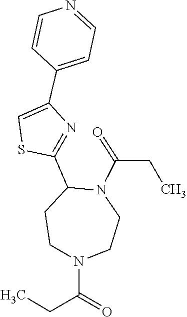 [SMILES: CCC(=O)N1CCC(c2nc(-c3ccncc3)cs2)N(C(=O)CC)CC1] | ¹H NMR (400 MHz, MeOH-d₄) δ 1.06-1.26 (m, 6H), 2.38-2.71 (m, 6H), 3.09-3.30 (m, 0.5H), 3.43-3.72 (m, 2.5H), 3.88-4.44 (m, 3H), 5.61-5.70 (m, 0.5H), 5.95-6.08 (m, 0.5H), 7.93-8.03 (m, 2H), 8.17 (s, 0.25H), 8.19 (s, 0.34H), 8.25 (s, 0.14H), 8.26 (s, 0.21H), 8.53-8.64 (m, 2H). | 373.0 (M + 1) |
| 102 | 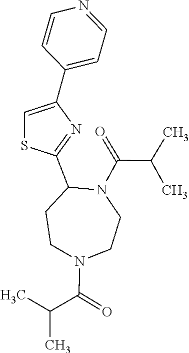 [SMILES: CC(C)C(=O)N1CCC(c2nc(-c3ccncc3)cs2)N(C(=O)C(C)C)CC1] | ¹H NMR (400 MHz, MeOH-d₄) δ 1.06-1.24 (m, 12H), 2.42-2.81 (m, 2H), 2.86-3.21 (m, 2.5H), 3.47-3.79 (m, 2.5H), 3.83-4.41 (m, 3H), 5.72-5.79 (m, 0.5H), 5.96-6.07 (m, 0.5H), 7.93-8.03 (m, 2H), 8.19 (s, 0.27H), 8.20 (s, 0.35H), 8.27 (s, 0.12H), 8.28 (s, 0.24H), 8.53-8.65 (m, 2H). | 401.4 (M + 1) |

Note:
¹H NMR data indicates these compounds exists as rotamers due to the presence of two tertiary amides. In some cases, enantiomers are separated but absolute stereochemistry for each enantiomer is not yet assigned.

### 4. Fatty Acid Uptake Assay for Activity Determination and Efficacy Study in SOD1 G93A Mouse Model of ALS

### 4.1 In Vitro—Fatty Acid Uptake Assay

### 4.1.1 Description

HEK293 cells were thawed and centrifuged at 400 g in 1 min, whereafter the supernatant was discarded. The cells were resuspended in DMEM+GlutaMax medium (cat #10566016, Invitrogen) containing 10% fetal calf serum (cat #10270-106, Invitrogen) and 1% penicillin/streptomycin (cat #15140-122, Life Technologies). HEK293 cells were seeded into a T25 flask and incubated at 37 degrees Celsius until 80% confluency. When the HEK293 cells reached confluency, cells were counted and seeded in a 96-well plate and with a volume of 10.000 cells/well in medium (as described above) and incubated overnight. Three wells were without cells serving as controls.

The cells were divided into two groups: 1) Example 1, and 2) Untreated. The fatty acid uptake assay (cat #408-100, BioVision) was carried out according to the protocol from BioVision. The only exception to the protocol was that the measurements in the PerkinElmer Multimode Plate Reader Enspire instrument was running overnight.

### 4.1.2 Statistics

Statistics was performed in GraphPad Prism version 8.0. Repeated measure two-way ANOVA were conducted as the groups were tested multiple times followed by a multiple comparison Bonferroni post hoc test.

### 4.1.3 Results of Fatty Acid Uptake Assay

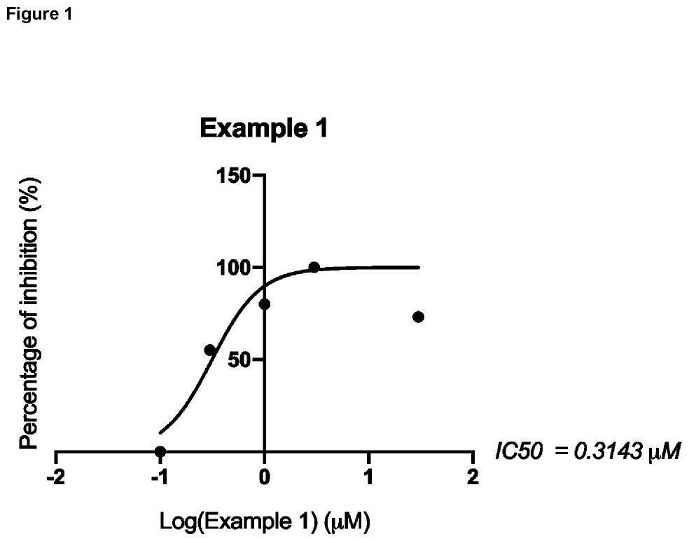

FIG. 1 shows the efficacy of the CPT1 inhibitors, Example 1 (Ex. 1, racemic mixture), tested in the fatty acid uptake assay using HEK293 cells with IC₅₀ of 0.3 μM.

### 4.2 In Vivo—SOD1 G93A Mouse Model of ALS

### 4.2.1 Description

### Animals

All experiments were approved by the Danish Animal Experiment Inspectorate (2017-15-0202-00088) and followed the National and European guidelines for conducting animal experiments. Animal experiments was conducted according to the ARRIVE guidelines. Mice were housed in IVC cages in a high barrier facility at Aalborg University, Denmark with a room temperature of 21° C. The mice were kept at a 12-hour light/dark cycle and had ad libitum access to food and water.

### SOD1 G93A Mouse Model

B6.Cg-Tg(SOD1*G93A)1Gur/J mice (Stock #004435) (SOD1) were purchased from Jackson Laboratory (Bar Harbor, USA). The congenic SOD1 mice were maintained in our animal facility by crossing hemizygote SOD1 male mice with female C₅₇BI/6J mice. Litters were genotyped according to established protocol using DNA extracted from ear tissue punches. Male SOD1 mice were used to maintain the colony, and SOD1 female and their wildtype littermates were used for experiments. All animals were assessed for human endpoints daily, weight twice a week, and neurological score once a week. SOD1 females (n=4) were evaluated at baseline and day 128 for cylinder, dark-light and y-maze tests, and at baseline and day 142 by neurological score, grip strength, hangwire test and rotarod. C57BI/6J female mice was used as placebo (n=8) receiving PBS was tested correspondingly. In addition, survival was evaluated as described under neurological score, onset of disease and survival analysis.

### Clinical Behavioral Tests

All clinical behavioral tests were performed between 9 am and 2 μm. All tests were performed in the same laboratory and mice were placed in the room one hour before test sessions to allow time for acclimatization. Test equipment was cleaned with 70% ethanol between each animal.

### Neurological Score, Onset of Disease and Survival Analysis

Mice were evaluated by the same experimenter weekly. The experimenter was blinded to treatment group and genotype. Mice were given a neurological score between zero to five as previously described.
Zero=no tremor in hindlimbs and full extension of hindlimbs when suspended by its tail.
One=tremor in hindlimbs and full extension of hindlimbs when suspended by its tail.
Two=tremor in hindlimbs and unable to extend hindlimbs when suspended by its tail.
Three=tremor in hindlimbs, unable to extend hindlimbs when suspended by its tail and wobbling gait.
Four=tremor in hindlimbs, unable to extend hindlimbs when suspended by its tail difficulty walking with paralysis of one of both hindlimbs.
Five=tremor in hindlimbs, unable to extend hindlimbs and unable to get up within 30 s when placed on its side.

Onset of disease was defined as the time point when tremor in the hind legs was present as previously described. Due to ethical reasons and based on the guidelines in the Animal Facility, mice were terminated if they reached a neurological score of four or latest at day 160. Based on this, survival was defined as a neurological score below 4 at the final day of experimentation. Due to the fact that mice had to be terminated at day 160, some mice did not reach a neurological score of 4 and therefore some of the groups have censored data in the survival analysis.

### Hangwire Test

Mice were gently placed on a wire grid lid and turn upside down. The latency to fall was noted. The maximum cut-off time was set to 180 s. Each mouse received three trials per sessions. The highest latency to fall was used for subsequent statistics.

### Rotarod Test

Rotarod test (Rotamex-5 RotaRod, Columbus Instruments, Columbus, Ohio, USA) was used with an acceleration from 4 to 40 RPM over 5 minutes. Mice were acclimatized to the rotarod over three consecutive days before the first test session. Each mouse was tested three times per test session to obtain a mean latency to fall (s).

### Grip Strength Test

Grip strength was evaluated using Grip strength meter (Bioseb, France). Briefly, the mouse was placed on a wire grid at pulled by its tail. The maximum tension was measured in grams Each mouse received 4 trials at each session and a mean grip strength was calculated. The mean grip strength was normalized to weight as previously described.

### Cylinder Test

The cylinder test was used to evaluate the sensorimotor function and spontaneous activity. Mice were transferred into a quiet room with low illumination and placed in the glass cylinders. Test was recorded for 3 minutes using a video camera. The number of rears were counted by four blinded raters.

### Y-Maze Test

The y-maze test was constructed according to Maze Engineers (USA). The mice were placed in the y-maze for 5 minutes to freely explore the three arms. The y-maze test was recorded by video and the number of entries and triplets were noted by blinded raters. The mean spontaneous alternation percentage was calculated.

### Dark Light Test

The dark-light test is used to measure the anxiety-like behaviour in mice. The mice were placed on the light-side in the box and freely explored this box for 5 minutes. The dark-light test was recorded by video, and the time to enter the dark as well as the time spent in the dark were noted by blinded raters.

### 4.2.2 Statistics

Statistics was performed in GraphPad Prism version 8.0. Repeated measure two-way ANOVA were conducted as the groups were tested multiple times followed by a multiple comparison Bonferroni post hoc test.

### 4.2.3 Results of the Efficacy Study in the SOD1 G93A Mouse Model of ALS

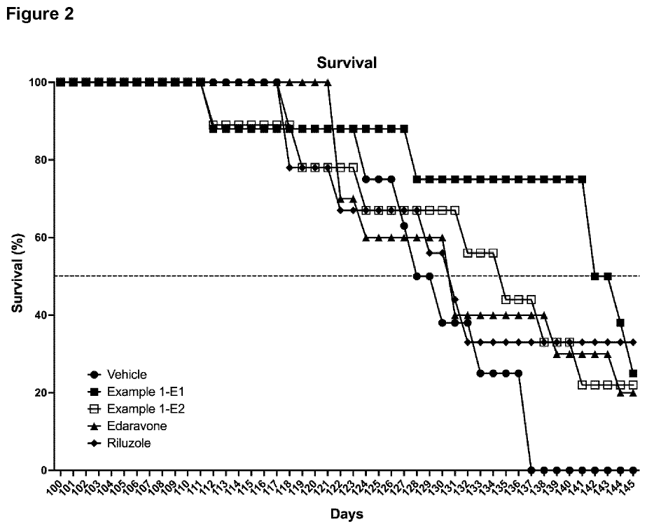

FIG. 2 shows the efficacy of the CPT1 inhibitor by improved survival of Example 1-E1 (Ex. 1) (n=10) and Example 1-E2 (Exp. 1) (n=10) in SOD1 G93A mice compared to SOD1 G93A mice receiving either vehicle (n=9), Edaravone (n=10) and Riluzole (n=10).

1. A compound of the formula (I) 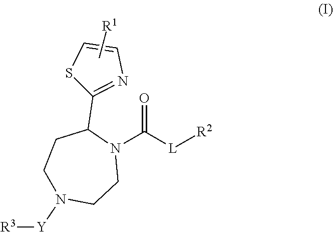
wherein
R¹ is unsubstituted or substituted aryl, preferably unsubstituted or substituted phenyl, naphthyl, tetrahydronaphthyl, indenyl, indanyl, pentalenyl, or fluorenyl,
unsubstituted or substituted heteroaryl, preferably unsubstituted or substituted pyridyl, pyrazinyl, pyrimidinyl, pyridazinyl, 2-oxo-1,2-dihydropyridinyl, oxazolyl, oxydiazolyl, isoxazolyl, thiadiazolyl, tetrazolyl, pyrazolyl, imidazolyl, thiazolyl and thienyl, quinolinyl, isoquinolinyl, cinnolinyl, pyrazolo[1,5-a]pyridyl, imidazo[1,2-a]pyridyl, quinoxalinyl, benzothiazolyl, benzotriazolyl, indolyl, or indazolyl,
unsubstituted or substituted 5- or 6-membered saturated or partially unsaturated heterocyclyl, or
unsubstituted or substituted C₃-C₈-cycloalkyl, or cyclohexenyl;
L is a single bond, a difunctional linker, preferably *—O—, *—OCH₂—, *—CH₂O—, *—CH₂—, *—CH₂—CH₂—, *—CH₂—CH₂—CH₂—, or *—CH₂—C(CH₃)₂—, or a trifunctional linker, preferably, *—CH═, wherein the * indicates the point of attachment to the carbonyl (C═O) group;
R² is unsubstituted or substituted phenyl, naphthyl, or pyridyl, or C₁-C₄-alkyl;
R³ is H, C₁-C₆-alkyl, halogen-C₁-C₄-alkyl, or C₃-C₈-cycloalkyl,
unsubstituted or substituted 4, 5- or 6-membered saturated or partially unsaturated heterocyclyl, or
unsubstituted or substituted phenyl;
Y is —(C═O)—, —(SO₂)— or a single bond;
or a stereoisomer, a pharmaceutically or veterinarily acceptable salt, hydrate or solvate thereof.

2. The compound of claim 1, wherein
R¹ is unsubstituted or substituted phenyl, naphthyl, or tetrahydronaphthyl,
unsubstituted or substituted pyridyl, pyrazinyl, pyrimidinyl, oxazolyl, oxydiazolyl, isoxazolyl, thiadiazolyl, tetrazolyl, pyrazolyl, imidazolyl, thiazolyl, thienyl, or quinolinyl,
unsubstituted or substituted pyrrolidinyl, piperidinyl, tetrahydropiperidinyl, or piperazinyl, or
unsubstituted or substituted cyclopentyl; cyclohexyl or cyclohexenyl;
L is a single bond, or a difunctional linker, preferably *—O—, *—OCH₂—, *—CH₂O—, *—CH₂—, or *—CH₂—CH₂—, wherein the * indicates the point of attachment to the carbonyl (C═O) group;
R² is unsubstituted or substituted phenyl, naphthyl, or pyridyl;
R³ is H, C₁-C₄-alkyl, halogen-C₁-C₄-alkyl, cyclopropyl, cyclobutyl, cyclopentyl or cyclohexyl,
unsubstituted or substituted azetidinyl, pyrrolidinyl, piperidinyl, or oxetanyl, 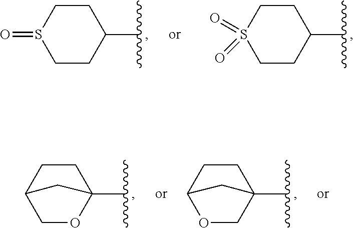 [SMILES: *C1CCS(=O)CC1 ; *C1CCS(=O)(=O)CC1 ; *C12CCC(CO1)C2 ; *C12CCC(C1)OC2]
unsubstituted or substituted phenyl; and
Y is —(C═O)—, —(SO₂)— or a single bond, preferably —(C═O)—.

3. The compound of claim 1, wherein
R¹ is unsubstituted or substituted phenyl, naphthyl, or tetrahydronaphthyl,
unsubstituted or substituted pyridyl, pyrazinyl, pyrimidinyl, oxazolyl, oxydiazolyl, isoxazolyl, thiadiazolyl, tetrazolyl, pyrazolyl, imidazolyl, thiazolyl, thienyl, or quinolinyl,
unsubstituted or substituted pyrrolidinyl, piperidinyl, tetrahydropiperidinyl, or piperazinyl, or
unsubstituted or substituted cyclopentyl, cyclohexyl or cyclohexenyl,
each R¹ being optionally and independently substituted with one or more, preferably with one of the following residues:
—CN,
halogen, preferably —F or —Cl,
C₁-C₄-alkyl, preferably methyl,
halogen-C₁-C₄-alkyl, preferably difluoromethyl, or trifluoromethyl,
SO₂Me, or
CO₂C₁-C₄-alkyl, preferably CO₂Me.

4. The compound of claim 1, wherein
R¹ is unsubstituted or substituted phenyl,
unsubstituted or substituted pyridyl, pyrazolyl, thienyl, or quinolinyl,
unsubstituted or substituted piperidinyl, or tetrahydropiperidinyl, or
unsubstituted or substituted cyclohexyl or cyclohexenyl;
each R¹ being optionally and independently substituted with one or more, preferably with one of the following residues:
—CN,
—F or —Cl,
C₁-C₄-alkyl, preferably methyl,
halogen-C₁-C₄-alkyl, preferably trifluoromethyl,
SO₂Me, or
CO₂C₁-C₄-alkyl, preferably CO₂Me.

5. The compound of claim 1, wherein
R¹ is unsubstituted or substituted phenyl or pyridyl,
being optionally and independently substituted with one or more, preferably with one of the following residues:
—F or —Cl,
C₁-C₄-alkyl, preferably methyl,
trifluoromethyl,
SO₂Me, or
CO₂C₁-C₄-alkyl, preferably CO₂Me.

6. The compound of claim 1, wherein
L is a single bond, *—CH₂O—, or *—CH₂—, preferably *—CH₂O—, wherein the * indicates the point of attachment to the carbonyl (C═O) group.

7. The compound of claim 1, wherein
R² is unsubstituted or substituted phenyl, naphthyl, or pyridyl; preferably phenyl,
each R² being optionally and independently substituted with one or more, preferably with one of the following residues:
—CN,
halogen, preferably —F or —Cl,
C₁-C₄-alkyl, preferably methyl,
halogen-C₁-C₄-alkyl, preferably difluoromethyl, or trifluoromethyl,
SO₂Me,
CO₂C₁-C₄-alkyl, preferably CO₂Me,
adamantyl,
unsubstituted or substituted phenyl, being optionally substituted with one or more, preferably with one substituent selected from
halogen, preferably —F or —Cl,
halogen-C₁-C₄-alkyl, preferably trifluoromethyl,
C₃-C₈-cycloalkyl, preferably cyclohexyl, or
pyridyl.

8. The compound of claim 1, wherein
R³ is H, C₁-C₄-alkyl, halogen-C₁-C₄-alkyl, cyclopropyl, cyclobutyl, cyclopentyl or cyclohexyl,
unsubstituted or substituted azetidinyl, pyrrolidinyl, piperidinyl, or oxetanyl,
unsubstituted or substituted phenyl.
each azetidinyl, pyrrolidinyl, piperidinyl, oxetanyl, or phenyl being optionally and independently substituted with one or more, preferably with one of the following residues:
—CN,
halogen, preferably —F or —Cl;
C₁-C₄-alkyl, preferably methyl;
halogen-C₁-C₄-alkyl, preferably chloromethyl, dichloromethyl, trichloromethyl, fluoromethyl, difluoromethyl, trifluoromethyl, chlorofluoromethyl, dichlorofluoromethyl, chlorodifluoromethyl, 1-fluoroethyl, 2-fluoroethyl, 2,2-difluoroethyl, 2,2,2-trifluoroethyl, 2-chloro-2fluoroethyl, 2-chloro-2,2-difluoroethyl, 2,2-dichloro-2-fluoroethyl, 2,2,2-trichloroethyl, pentafluoroethyl, or 2,2-difluor-3-methyl-butyl, particularly preferred difluoromethyl, or trifluoromethyl,
SO₂Me,
CO₂C₁-C₄-alkyl, preferably CO₂Me, or
CO—C₁-C₄-alky, preferably CO-Me.

9. The compound of claim 1, wherein
R³ is C₁-C₄-alkyl, preferably methyl.

10. The compound of claim 1, selected from:

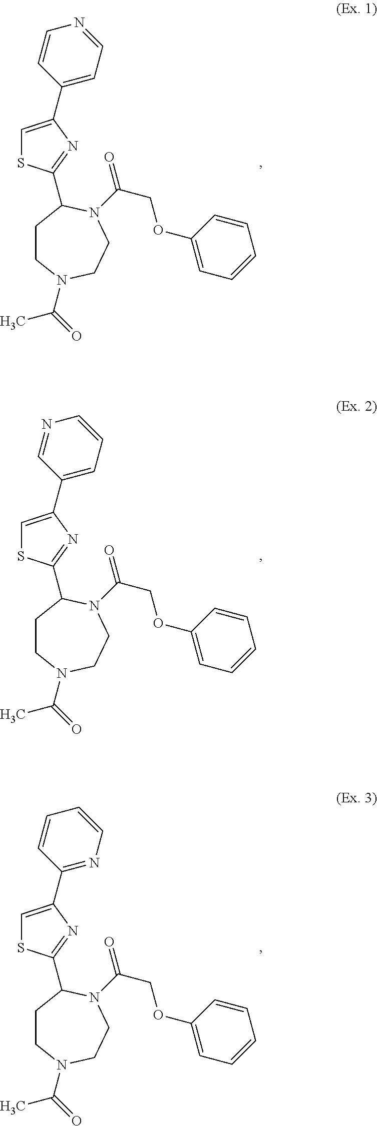 [SMILES: CC(=O)N1CCC(c2nc(-c3ccncc3)cs2)N(C(=O)COc2ccccc2)CC1 ; CC(=O)N1CCC(c2nc(-c3cccnc3)cs2)N(C(=O)COc2ccccc2)CC1 ; CC(=O)N1CCC(c2nc(-c3ccccn3)cs2)N(C(=O)COc2ccccc2)CC1]

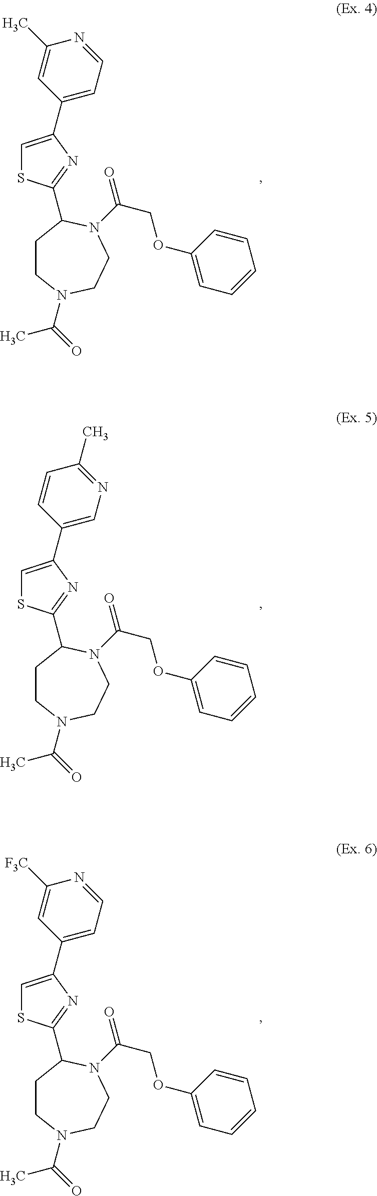 [SMILES: CC(=O)N1CCC(c2nc(-c3ccnc(C)c3)cs2)N(C(=O)COc2ccccc2)CC1 ; CC(=O)N1CCC(c2nc(-c3ccc(C)nc3)cs2)N(C(=O)COc2ccccc2)CC1 ; CC(=O)N1CCC(c2nc(-c3ccnc(C(F)(F)F)c3)cs2)N(C(=O)COc2ccccc2)CC1]

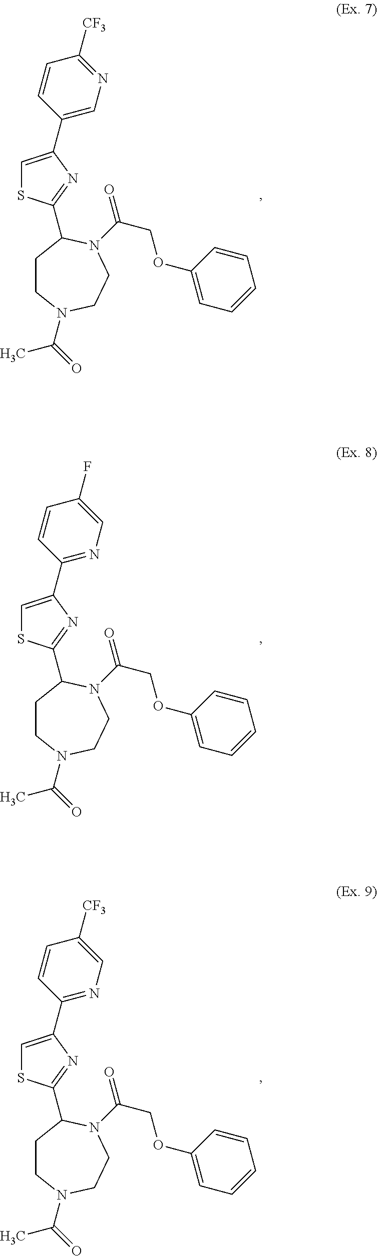 [SMILES: CC(=O)N1CCC(c2nc(-c3ccc(C(F)(F)F)nc3)cs2)N(C(=O)COc2ccccc2)CC1 ; CC(=O)N1CCC(c2nc(-c3ccc(F)cn3)cs2)N(C(=O)COc2ccccc2)CC1 ; CC(=O)N1CCC(c2nc(-c3ccc(C(F)(F)F)cn3)cs2)N(C(=O)COc2ccccc2)CC1]

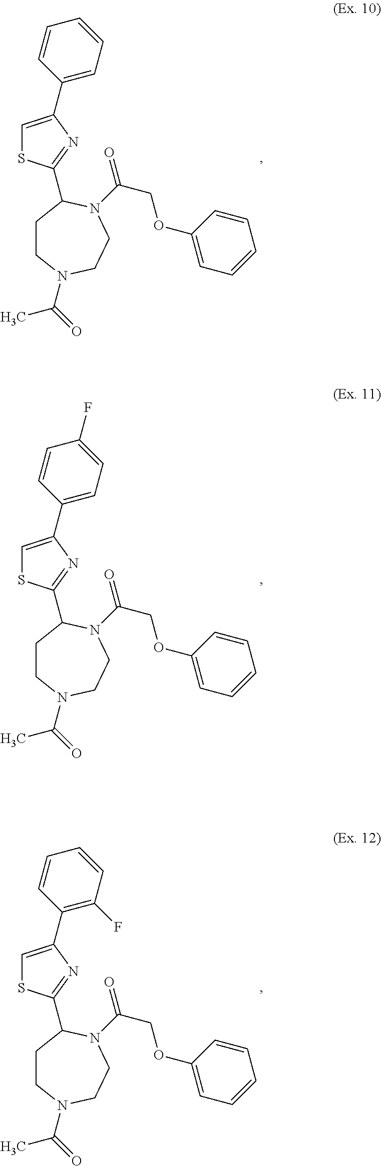 [SMILES: CC(=O)N1CCC(c2nc(-c3ccccc3)cs2)N(C(=O)COc2ccccc2)CC1 ; CC(=O)N1CCC(c2nc(-c3ccc(F)cc3)cs2)N(C(=O)COc2ccccc2)CC1 ; CC(=O)N1CCC(c2nc(-c3ccccc3F)cs2)N(C(=O)COc2ccccc2)CC1]

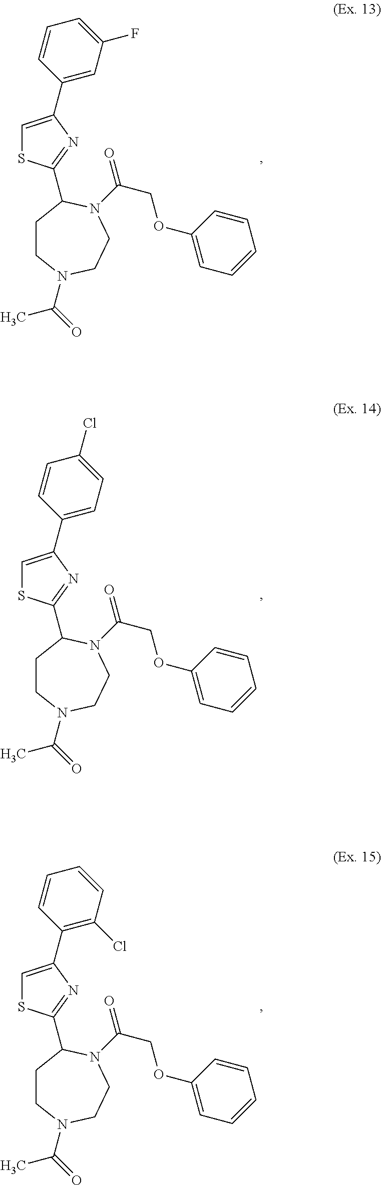 [SMILES: CC(=O)N1CCC(c2nc(-c3cccc(F)c3)cs2)N(C(=O)COc2ccccc2)CC1 ; CC(=O)N1CCC(c2nc(-c3ccc(Cl)cc3)cs2)N(C(=O)COc2ccccc2)CC1 ; CC(=O)N1CCC(c2nc(-c3ccccc3Cl)cs2)N(C(=O)COc2ccccc2)CC1]

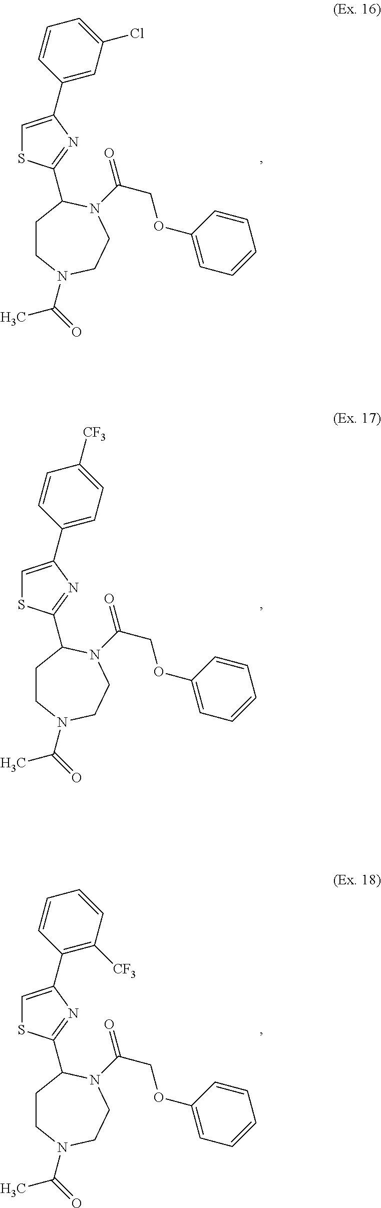 [SMILES: CC(=O)N1CCC(c2nc(-c3cccc(Cl)c3)cs2)N(C(=O)COc2ccccc2)CC1 ; CC(=O)N1CCC(c2nc(-c3ccc(C(F)(F)F)cc3)cs2)N(C(=O)COc2ccccc2)CC1 ; CC(=O)N1CCC(c2nc(-c3ccccc3C(F)(F)F)cs2)N(C(=O)COc2ccccc2)CC1]

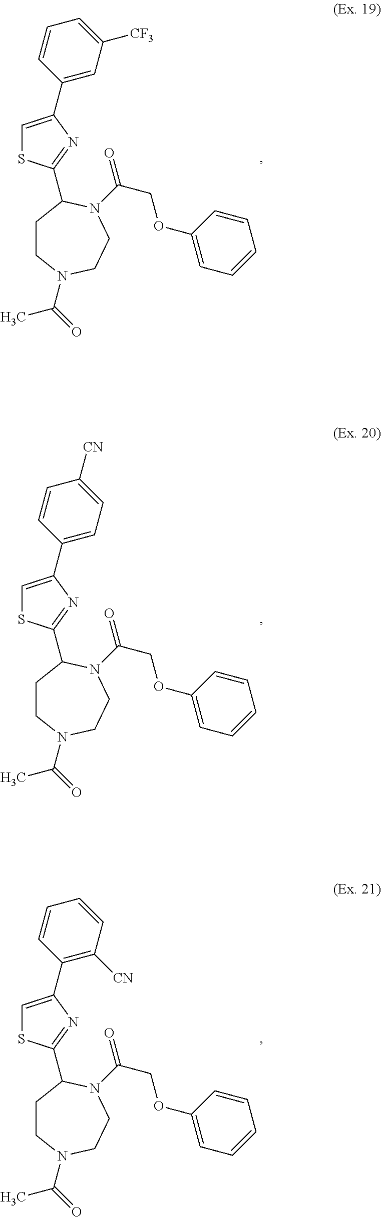 [SMILES: CC(=O)N1CCC(c2nc(-c3cccc(C(F)(F)F)c3)cs2)N(C(=O)COc2ccccc2)CC1 ; CC(=O)N1CCC(c2nc(-c3ccc(C#N)cc3)cs2)N(C(=O)COc2ccccc2)CC1 ; CC(=O)N1CCC(c2nc(-c3ccccc3C#N)cs2)N(C(=O)COc2ccccc2)CC1]

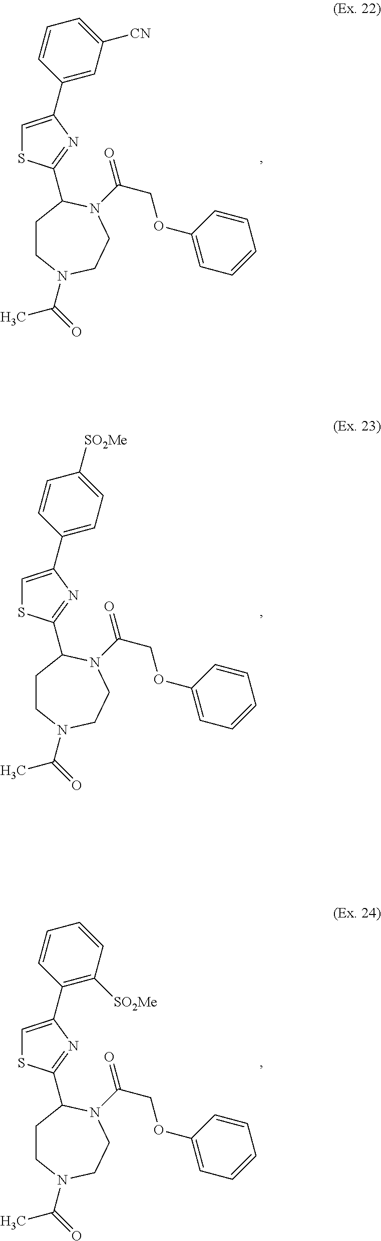 [SMILES: CC(=O)N1CCC(c2nc(-c3cccc(C#N)c3)cs2)N(C(=O)COc2ccccc2)CC1 ; CC(=O)N1CCC(c2nc(-c3ccc(S(C)(=O)=O)cc3)cs2)N(C(=O)COc2ccccc2)CC1 ; CC(=O)N1CCC(c2nc(-c3ccccc3S(C)(=O)=O)cs2)N(C(=O)COc2ccccc2)CC1]

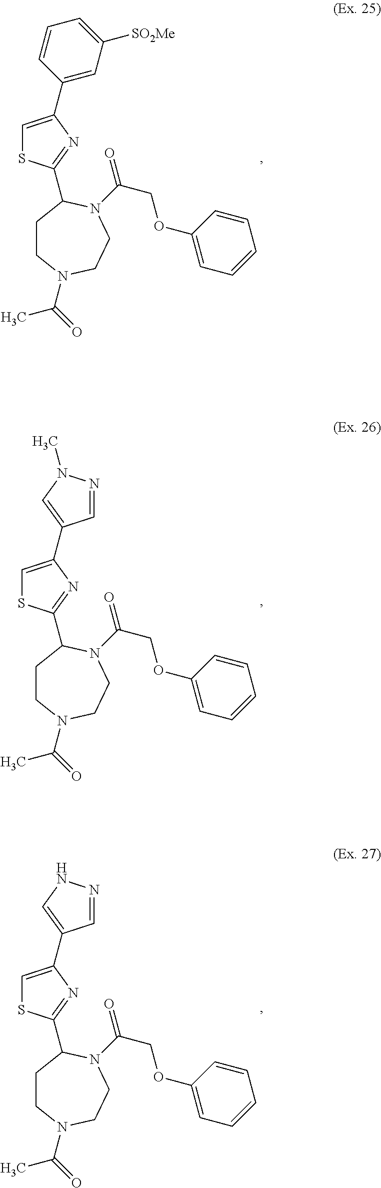 [SMILES: CC(=O)N1CCC(c2nc(-c3cccc(S(C)(=O)=O)c3)cs2)N(C(=O)COc2ccccc2)CC1 ; CC(=O)N1CCC(c2nc(-c3cnn(C)c3)cs2)N(C(=O)COc2ccccc2)CC1 ; CC(=O)N1CCC(c2nc(-c3cn[nH]c3)cs2)N(C(=O)COc2ccccc2)CC1]

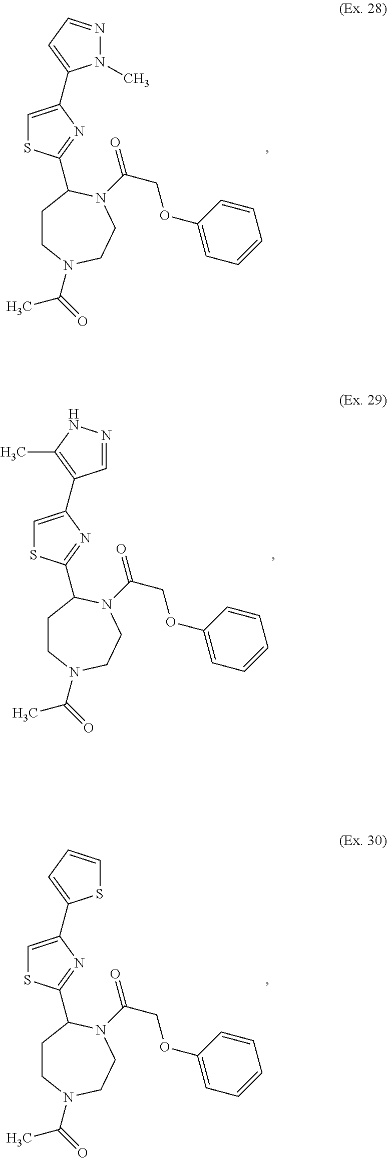 [SMILES: CC(=O)N1CCC(c2nc(-c3ccnn3C)cs2)N(C(=O)COc2ccccc2)CC1 ; CC(=O)N1CCC(c2nc(-c3cn[nH]c3C)cs2)N(C(=O)COc2ccccc2)CC1 ; CC(=O)N1CCC(c2nc(-c3cccs3)cs2)N(C(=O)COc2ccccc2)CC1]

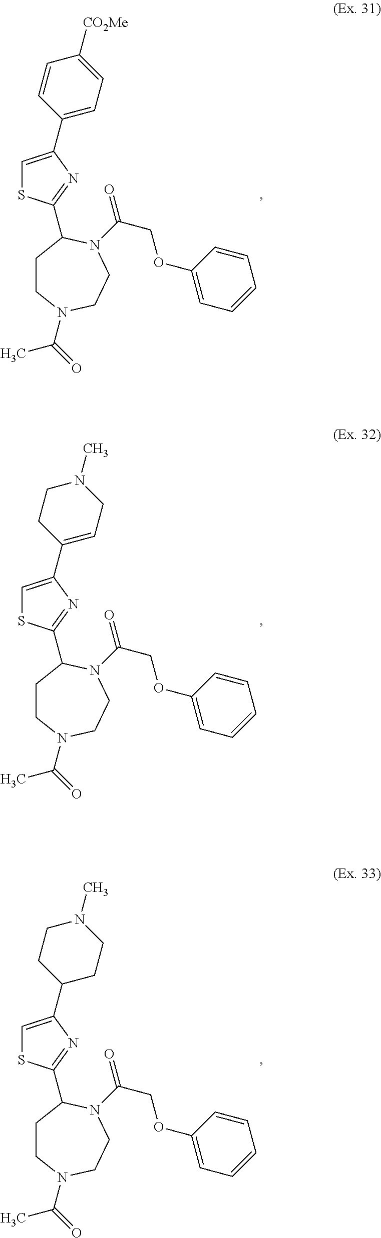 [SMILES: COC(=O)c1ccc(-c2csc(C3CCN(C(C)=O)CCN3C(=O)COc3ccccc3)n2)cc1 ; CC(=O)N1CCC(c2nc(C3=CCN(C)CC3)cs2)N(C(=O)COc2ccccc2)CC1 ; CC(=O)N1CCC(c2nc(C3CCN(C)CC3)cs2)N(C(=O)COc2ccccc2)CC1]

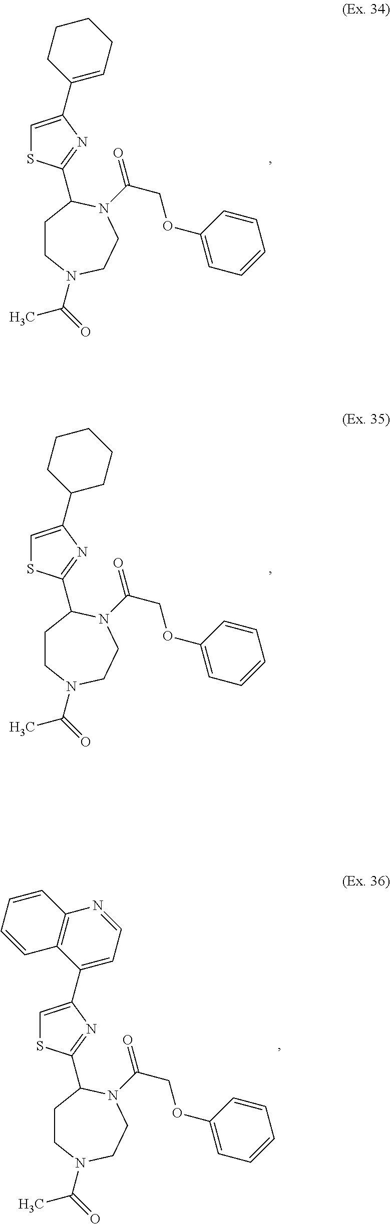 [SMILES: CC(=O)N1CCC(c2nc(C3=CCCCC3)cs2)N(C(=O)COc2ccccc2)CC1 ; CC(=O)N1CCC(c2nc(C3CCCCC3)cs2)N(C(=O)COc2ccccc2)CC1 ; CC(=O)N1CCC(c2nc(-c3ccnc4ccccc34)cs2)N(C(=O)COc2ccccc2)CC1]

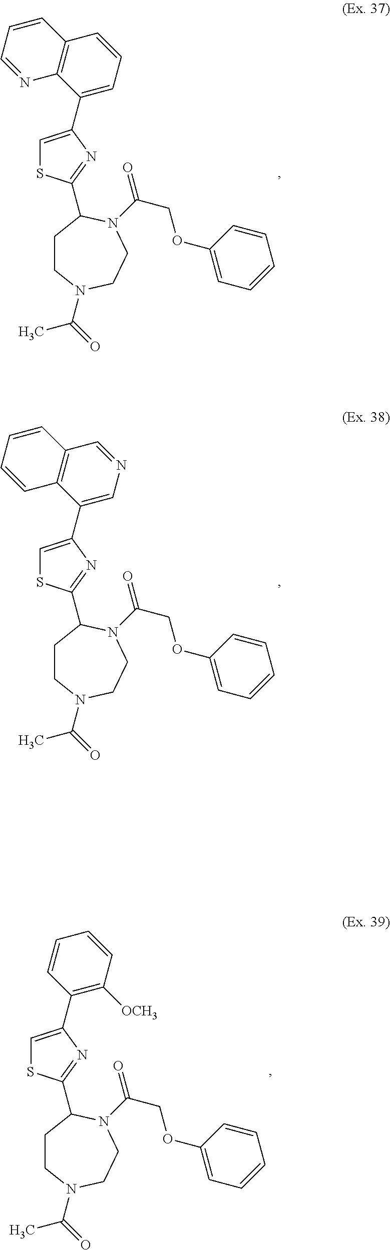 [SMILES: CC(=O)N1CCC(c2nc(-c3cccc4cccnc34)cs2)N(C(=O)COc2ccccc2)CC1 ; CC(=O)N1CCC(c2nc(-c3cncc4ccccc34)cs2)N(C(=O)COc2ccccc2)CC1 ; COc1ccccc1-c1csc(C2CCN(C(C)=O)CCN2C(=O)COc2ccccc2)n1]

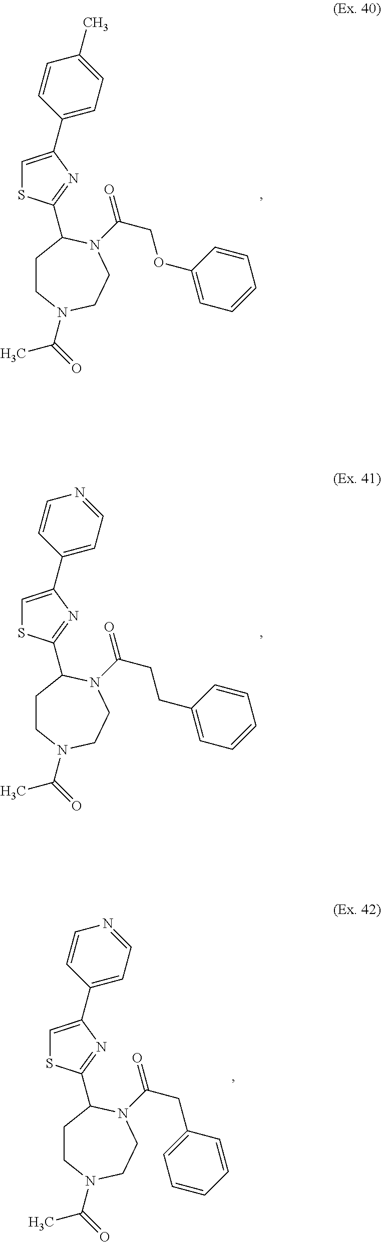 [SMILES: CC(=O)N1CCC(c2nc(-c3ccc(C)cc3)cs2)N(C(=O)COc2ccccc2)CC1 ; CC(=O)N1CCC(c2nc(-c3ccncc3)cs2)N(C(=O)CCc2ccccc2)CC1 ; CC(=O)N1CCC(c2nc(-c3ccncc3)cs2)N(C(=O)Cc2ccccc2)CC1]

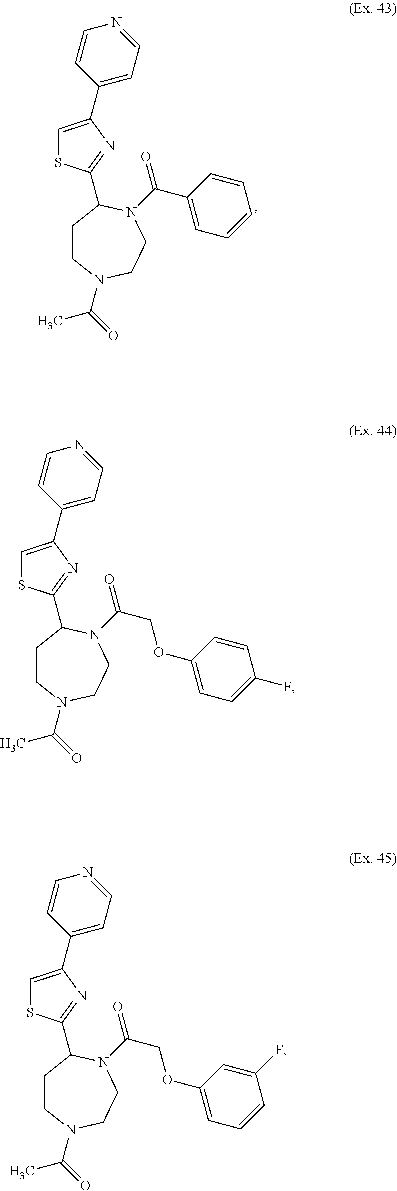 [SMILES: CC(=O)N1CCC(c2nc(-c3ccncc3)cs2)N(C(=O)c2ccccc2)CC1 ; CC(=O)N1CCC(c2nc(-c3ccncc3)cs2)N(C(=O)COc2ccc(F)cc2)CC1 ; CC(=O)N1CCC(c2nc(-c3ccncc3)cs2)N(C(=O)COc2cccc(F)c2)CC1]

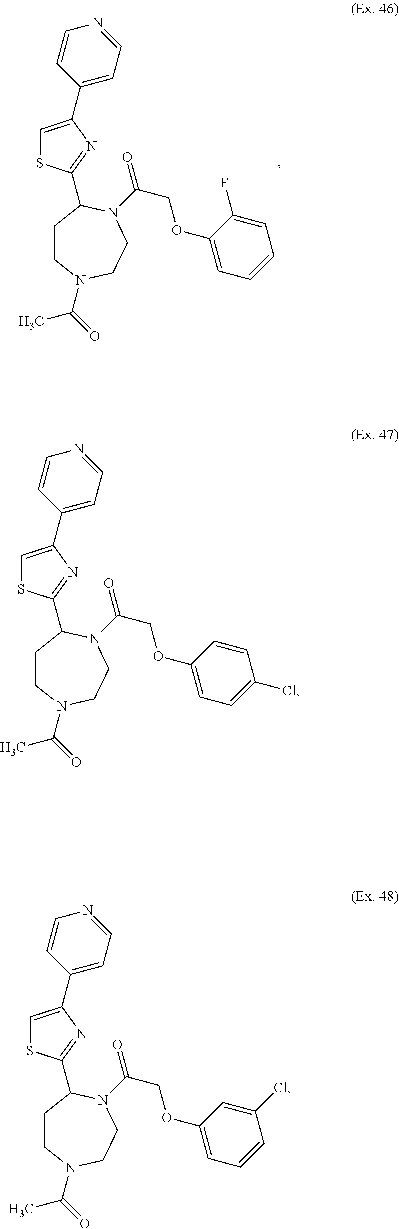 [SMILES: CC(=O)N1CCC(c2nc(-c3ccncc3)cs2)N(C(=O)COc2ccccc2F)CC1 ; CC(=O)N1CCC(c2nc(-c3ccncc3)cs2)N(C(=O)COc2ccc(Cl)cc2)CC1 ; CC(=O)N1CCC(c2nc(-c3ccncc3)cs2)N(C(=O)COc2cccc(Cl)c2)CC1]

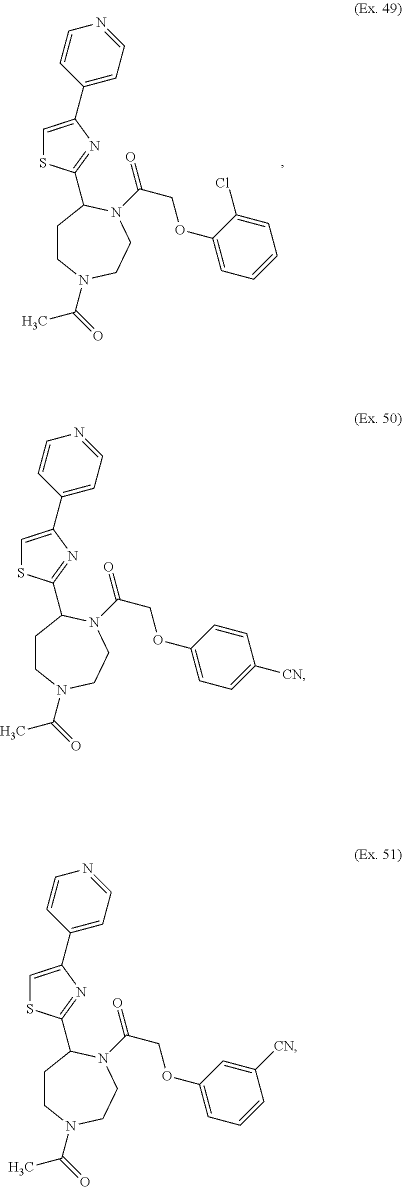 [SMILES: CC(=O)N1CCC(c2nc(-c3ccncc3)cs2)N(C(=O)COc2ccccc2Cl)CC1 ; CC(=O)N1CCC(c2nc(-c3ccncc3)cs2)N(C(=O)COc2ccc(C#N)cc2)CC1 ; CC(=O)N1CCC(c2nc(-c3ccncc3)cs2)N(C(=O)COc2cccc(C#N)c2)CC1]

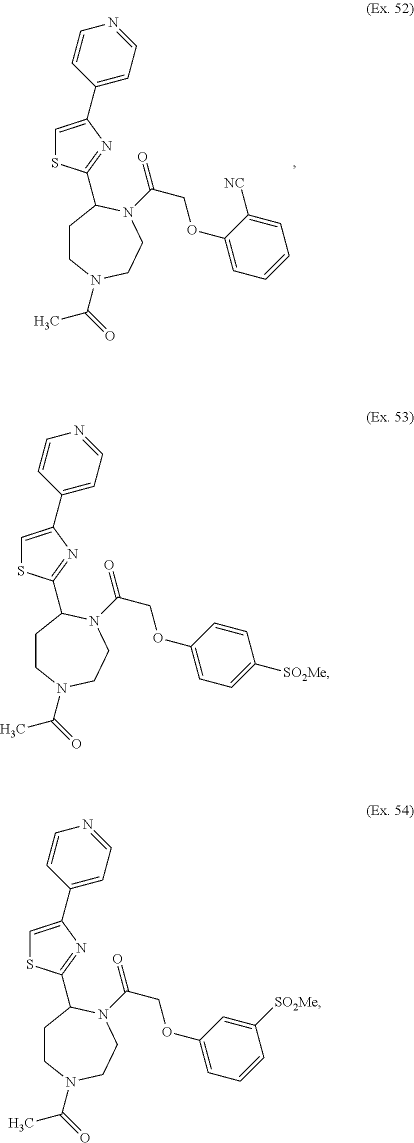 [SMILES: CC(=O)N1CCC(c2nc(-c3ccncc3)cs2)N(C(=O)COc2ccccc2C#N)CC1 ; CC(=O)N1CCC(c2nc(-c3ccncc3)cs2)N(C(=O)COc2ccc(S(C)(=O)=O)cc2)CC1 ; CC(=O)N1CCC(c2nc(-c3ccncc3)cs2)N(C(=O)COc2cccc(S(C)(=O)=O)c2)CC1]

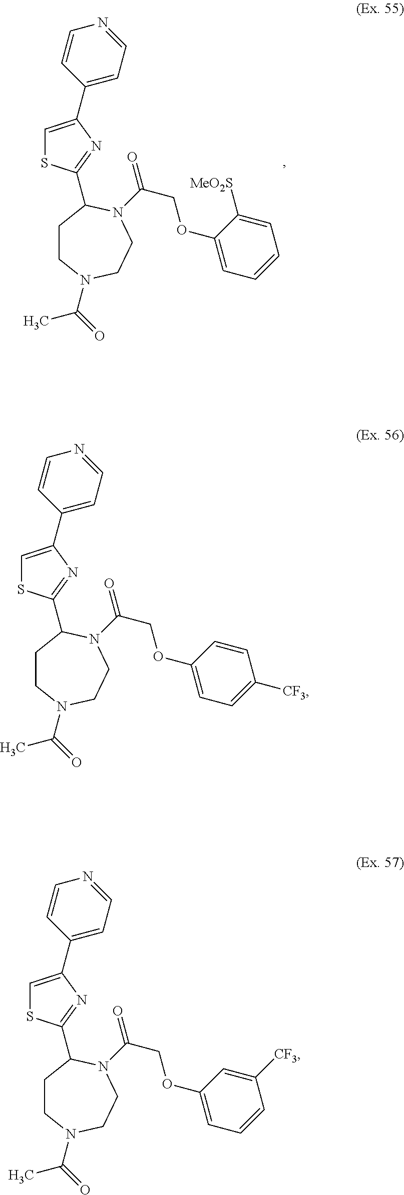 [SMILES: CC(=O)N1CCC(c2nc(-c3ccncc3)cs2)N(C(=O)COc2ccccc2S(C)(=O)=O)CC1 ; CC(=O)N1CCC(c2nc(-c3ccncc3)cs2)N(C(=O)COc2ccc(C(F)(F)F)cc2)CC1 ; CC(=O)N1CCC(c2nc(-c3ccncc3)cs2)N(C(=O)COc2cccc(C(F)(F)F)c2)CC1]

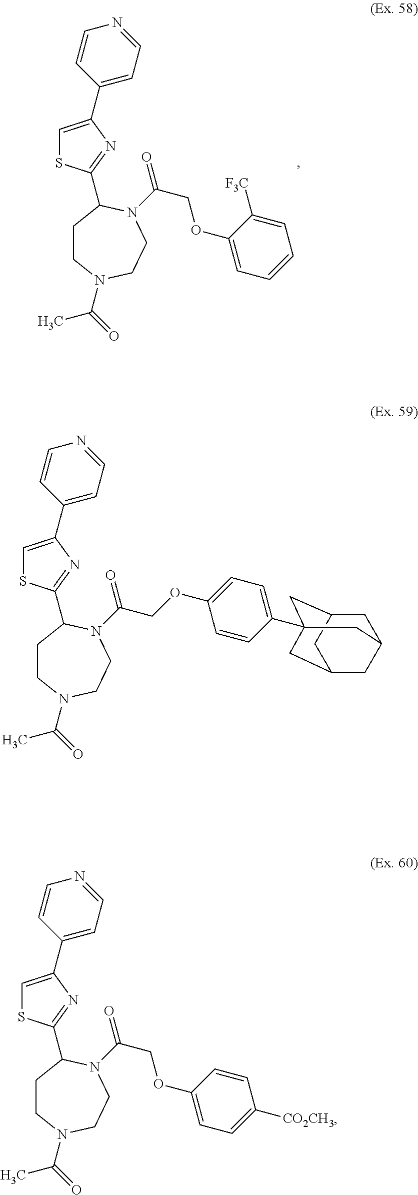 [SMILES: CC(=O)N1CCC(c2nc(-c3ccncc3)cs2)N(C(=O)COc2ccccc2C(F)(F)F)CC1 ; CC(=O)N1CCC(c2nc(-c3ccncc3)cs2)N(C(=O)COc2ccc(C34CC5CC(CC(C5)C3)C4)cc2)CC1 ; COC(=O)c1ccc(OCC(=O)N2CCN(C(C)=O)CCC2c2nc(-c3ccncc3)cs2)cc1]

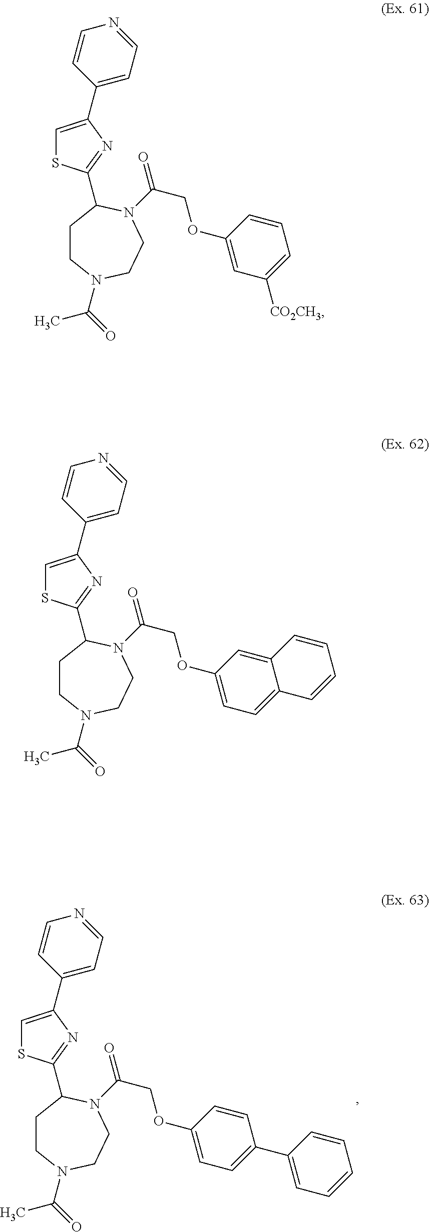 [SMILES: COC(=O)c1cccc(OCC(=O)N2CCN(C(C)=O)CCC2c2nc(-c3ccncc3)cs2)c1 ; CC(=O)N1CCC(c2nc(-c3ccncc3)cs2)N(C(=O)COc2ccc3ccccc3c2)CC1 ; CC(=O)N1CCC(c2nc(-c3ccncc3)cs2)N(C(=O)COc2ccc(-c3ccccc3)cc2)CC1]

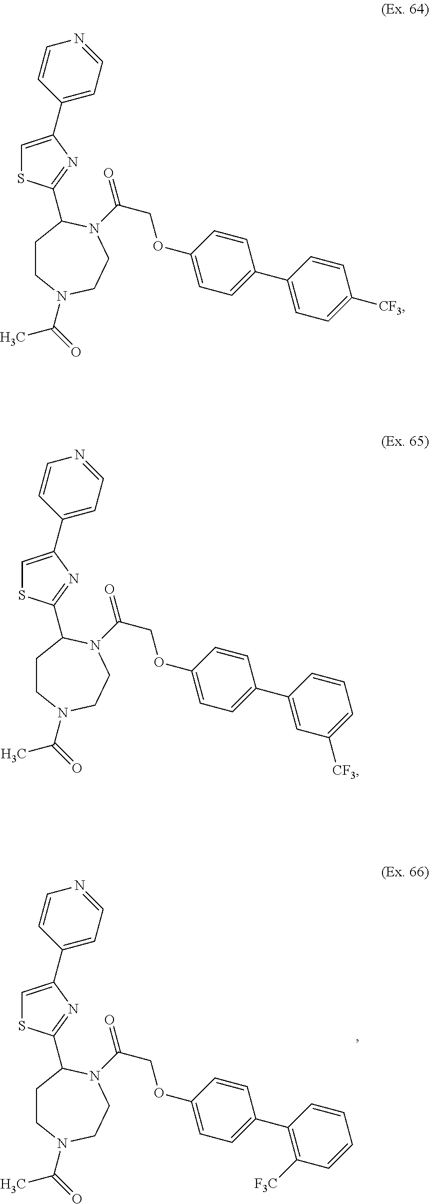 [SMILES: CC(=O)N1CCC(c2nc(-c3ccncc3)cs2)N(C(=O)COc2ccc(-c3ccc(C(F)(F)F)cc3)cc2)CC1 ; CC(=O)N1CCC(c2nc(-c3ccncc3)cs2)N(C(=O)COc2ccc(-c3cccc(C(F)(F)F)c3)cc2)CC1 ; CC(=O)N1CCC(c2nc(-c3ccncc3)cs2)N(C(=O)COc2ccc(-c3ccccc3C(F)(F)F)cc2)CC1]

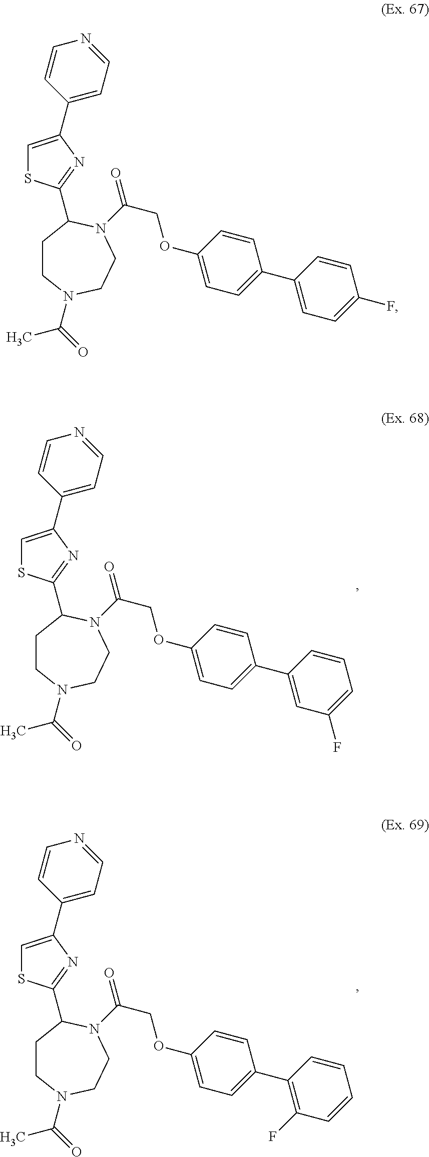 [SMILES: CC(=O)N1CCC(c2nc(-c3ccncc3)cs2)N(C(=O)COc2ccc(-c3ccc(F)cc3)cc2)CC1 ; CC(=O)N1CCC(c2nc(-c3ccncc3)cs2)N(C(=O)COc2ccc(-c3cccc(F)c3)cc2)CC1 ; CC(=O)N1CCC(c2nc(-c3ccncc3)cs2)N(C(=O)COc2ccc(-c3ccccc3F)cc2)CC1]

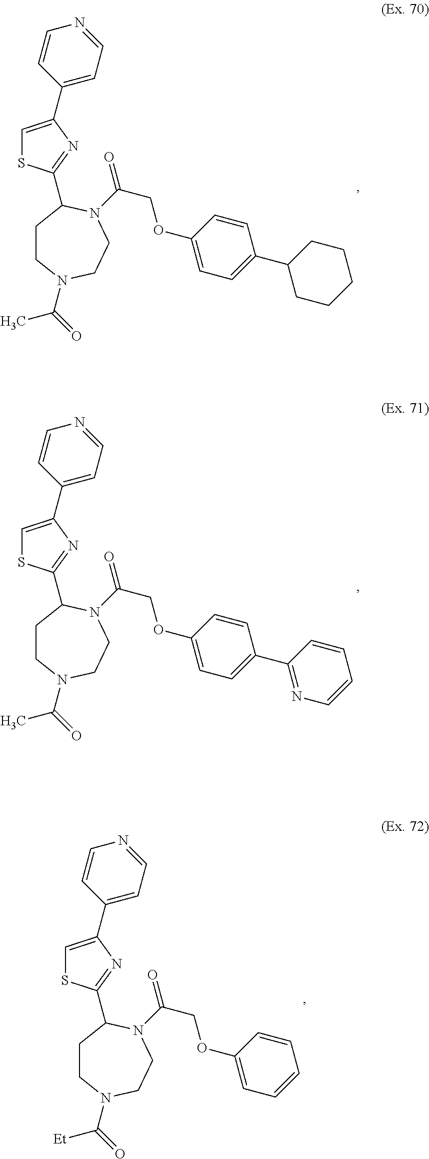 [SMILES: CC(=O)N1CCC(c2nc(-c3ccncc3)cs2)N(C(=O)COc2ccc(C3CCCCC3)cc2)CC1 ; CC(=O)N1CCC(c2nc(-c3ccncc3)cs2)N(C(=O)COc2ccc(-c3ccccn3)cc2)CC1 ; CCC(=O)N1CCC(c2nc(-c3ccncc3)cs2)N(C(=O)COc2ccccc2)CC1]

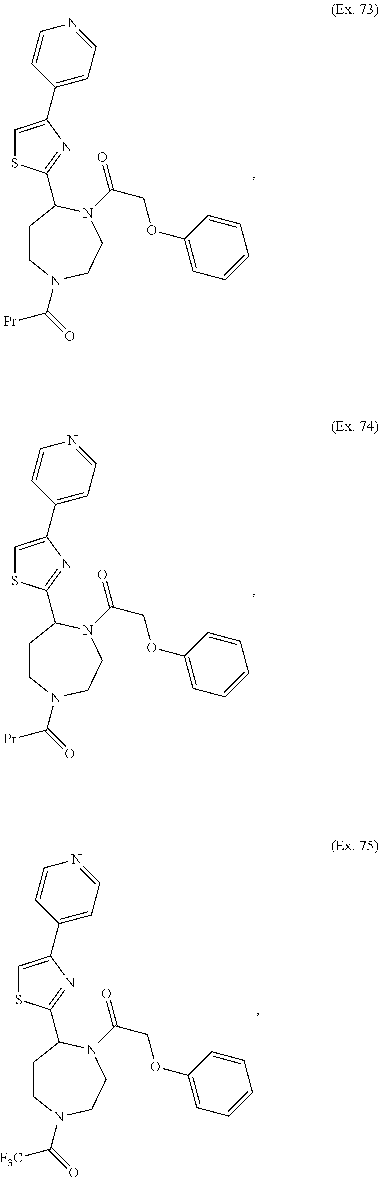

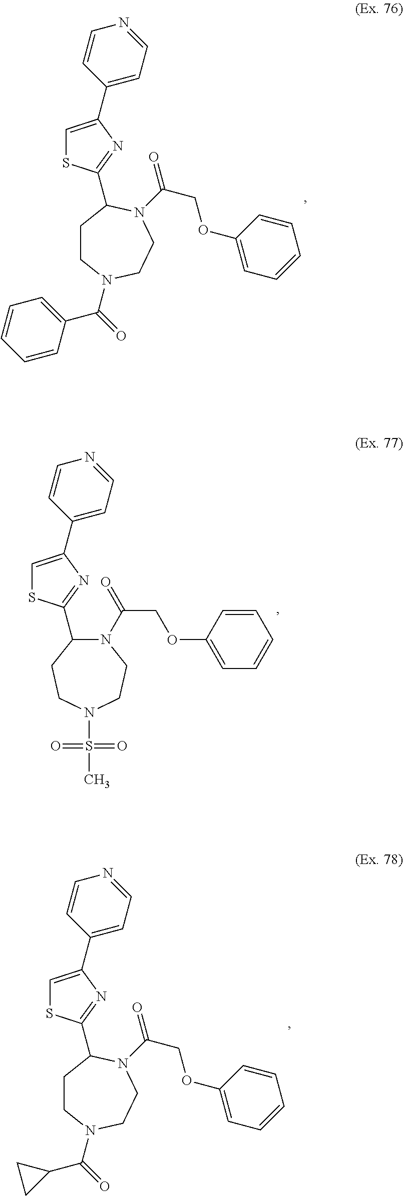 [SMILES: O=C(c1ccccc1)N1CCC(c2nc(-c3ccncc3)cs2)N(C(=O)COc2ccccc2)CC1 ; CS(=O)(=O)N1CCC(c2nc(-c3ccncc3)cs2)N(C(=O)COc2ccccc2)CC1 ; O=C(C1CC1)N1CCC(c2nc(-c3ccncc3)cs2)N(C(=O)COc2ccccc2)CC1]

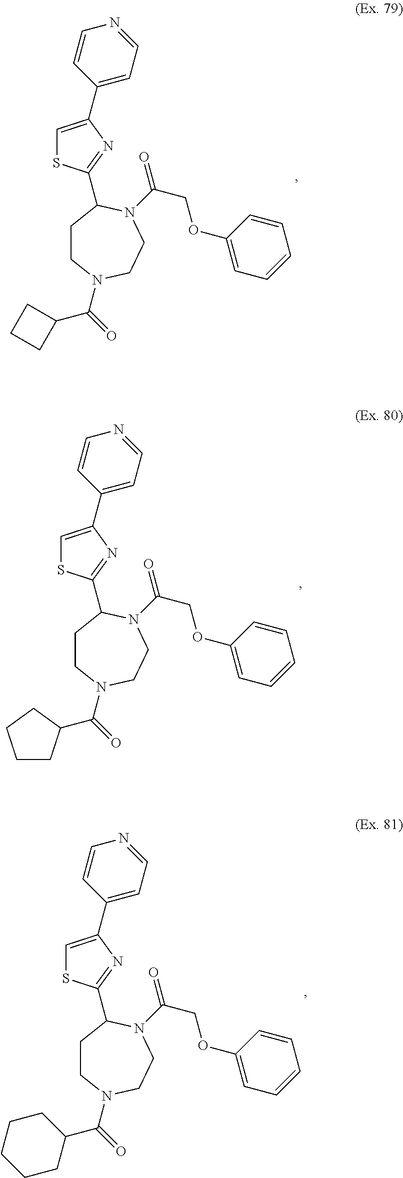 [SMILES: O=C(C1CCC1)N1CCC(c2nc(-c3ccncc3)cs2)N(C(=O)COc2ccccc2)CC1 ; O=C(C1CCCC1)N1CCC(c2nc(-c3ccncc3)cs2)N(C(=O)COc2ccccc2)CC1 ; O=C(C1CCCCC1)N1CCC(c2nc(-c3ccncc3)cs2)N(C(=O)COc2ccccc2)CC1]

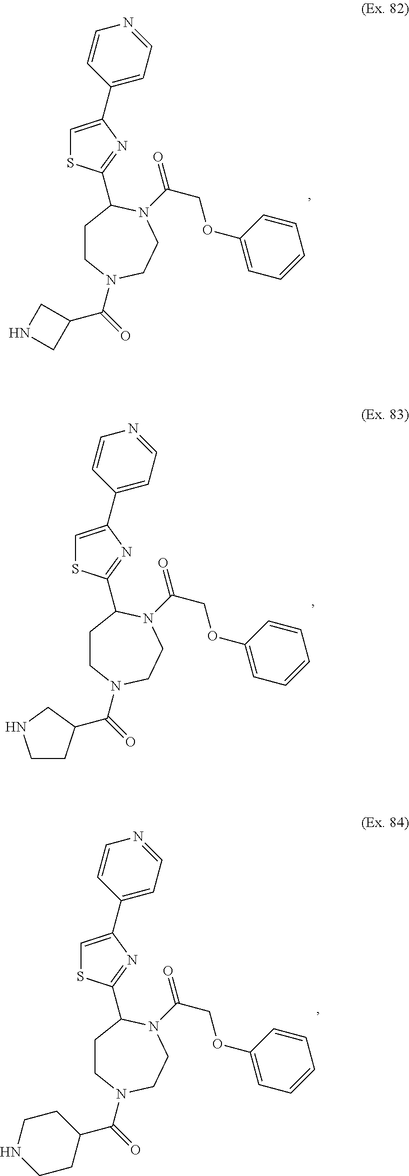 [SMILES: O=C(C1CNC1)N1CCC(c2nc(-c3ccncc3)cs2)N(C(=O)COc2ccccc2)CC1 ; O=C(C1CCNC1)N1CCC(c2nc(-c3ccncc3)cs2)N(C(=O)COc2ccccc2)CC1 ; O=C(C1CCNCC1)N1CCC(c2nc(-c3ccncc3)cs2)N(C(=O)COc2ccccc2)CC1]

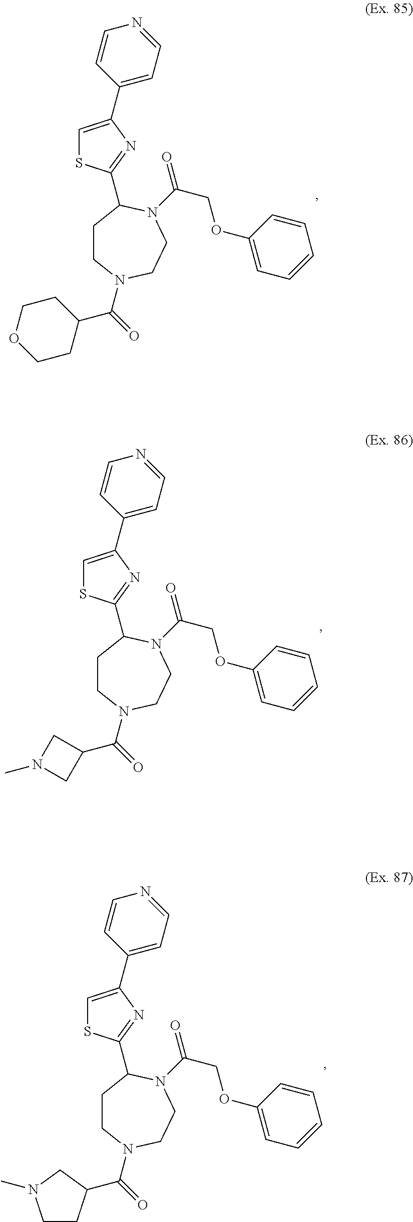 [SMILES: O=C(C1CCOCC1)N1CCC(c2nc(-c3ccncc3)cs2)N(C(=O)COc2ccccc2)CC1 ; CN1CC(C(=O)N2CCC(c3nc(-c4ccncc4)cs3)N(C(=O)COc3ccccc3)CC2)C1 ; CN1CCC(C(=O)N2CCC(c3nc(-c4ccncc4)cs3)N(C(=O)COc3ccccc3)CC2)C1]

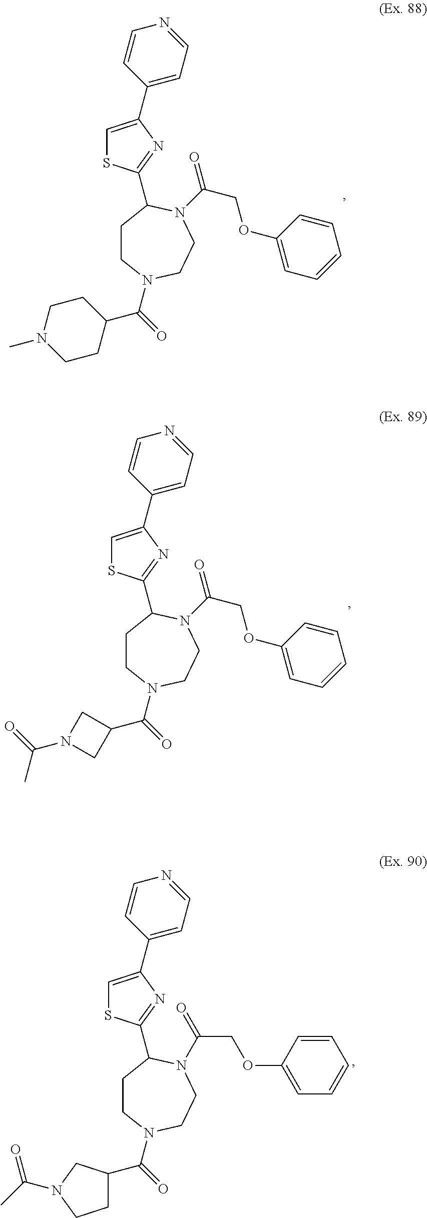 [SMILES: CN1CCC(C(=O)N2CCC(c3nc(-c4ccncc4)cs3)N(C(=O)COc3ccccc3)CC2)CC1 ; CC(=O)N1CC(C(=O)N2CCC(c3nc(-c4ccncc4)cs3)N(C(=O)COc3ccccc3)CC2)C1 ; CC(=O)N1CCC(C(=O)N2CCC(c3nc(-c4ccncc4)cs3)N(C(=O)COc3ccccc3)CC2)C1]

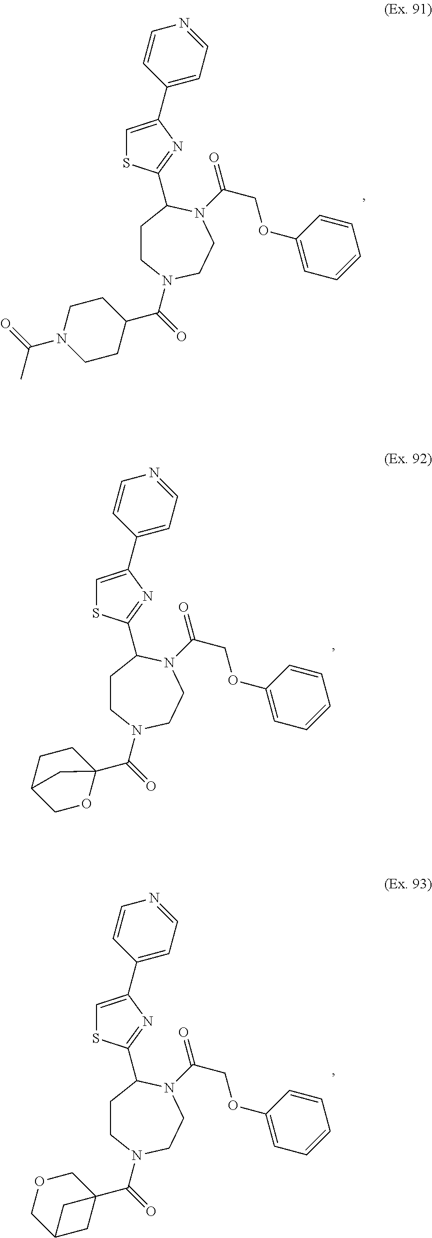 [SMILES: CC(=O)N1CCC(C(=O)N2CCC(c3nc(-c4ccncc4)cs3)N(C(=O)COc3ccccc3)CC2)CC1 ; O=C(COc1ccccc1)N1CCN(C(=O)C23CCC(CO2)C3)CCC1c1nc(-c2ccncc2)cs1 ; O=C(COc1ccccc1)N1CCN(C(=O)C23COCC(C2)C3)CCC1c1nc(-c2ccncc2)cs1]

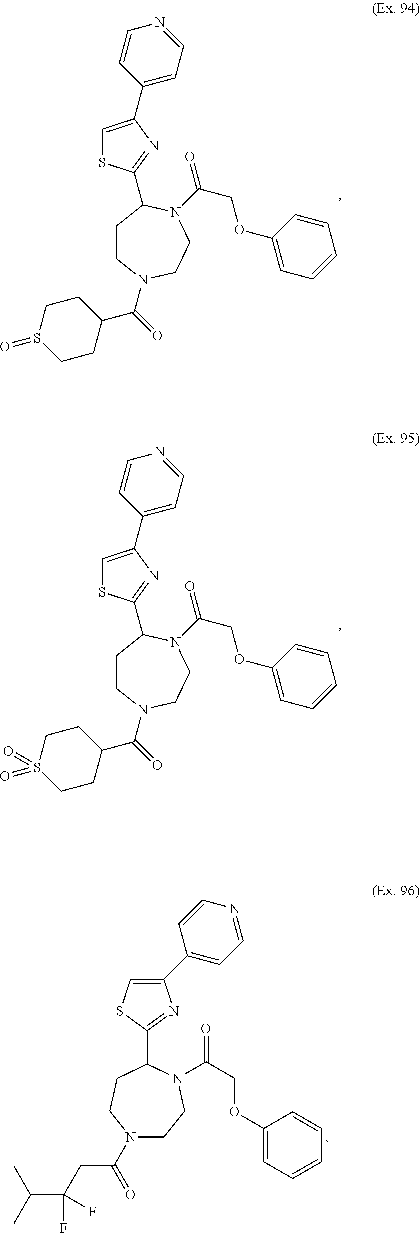 [SMILES: O=C(C1CCS(=O)CC1)N1CCC(c2nc(-c3ccncc3)cs2)N(C(=O)COc2ccccc2)CC1 ; O=C(C1CCS(=O)(=O)CC1)N1CCC(c2nc(-c3ccncc3)cs2)N(C(=O)COc2ccccc2)CC1 ; CC(C)C(F)(F)CC(=O)N1CCC(c2nc(-c3ccncc3)cs2)N(C(=O)COc2ccccc2)CC1]

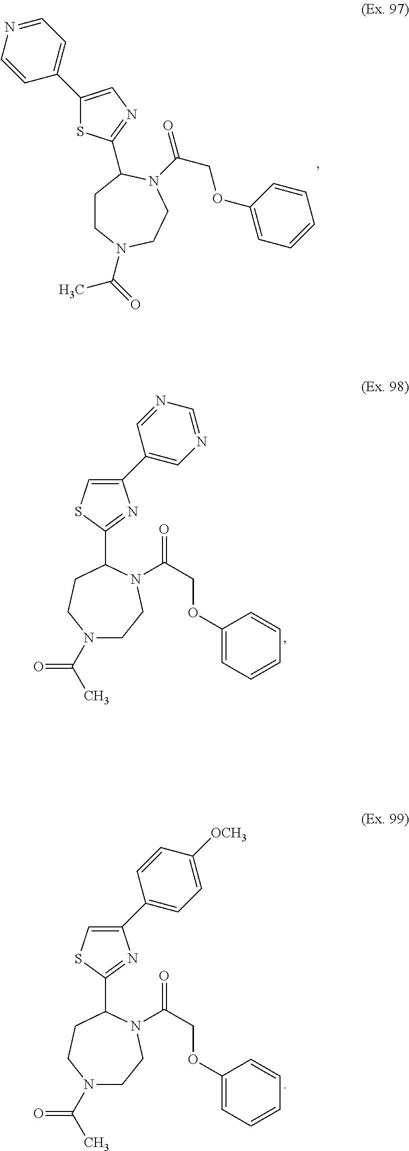 [SMILES: CC(=O)N1CCC(c2ncc(-c3ccncc3)s2)N(C(=O)COc2ccccc2)CC1 ; CC(=O)N1CCC(c2nc(-c3cncnc3)cs2)N(C(=O)COc2ccccc2)CC1 ; COc1ccc(-c2csc(C3CCN(C(C)=O)CCN3C(=O)COc3ccccc3)n2)cc1]

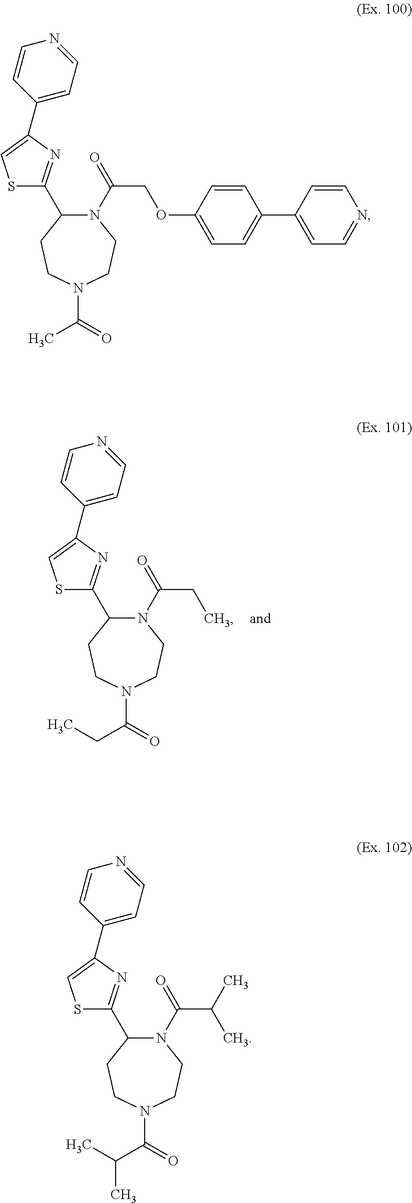 [SMILES: CC(=O)N1CCC(c2nc(-c3ccncc3)cs2)N(C(=O)COc2ccc(-c3ccncc3)cc2)CC1 ; CCC(=O)N1CCC(c2nc(-c3ccncc3)cs2)N(C(=O)CC)CC1 ; CC(C)C(=O)N1CCC(c2nc(-c3ccncc3)cs2)N(C(=O)C(C)C)CC1]

11. A compound as defined in claim 1 or a stereoisomer, a pharmaceutically or veterinarily acceptable salt, hydrate or solvate thereof for use as a medicament.

12. A pharmaceutical composition comprising a compound as defined in claim 1 or a stereoisomer, a pharmaceutically or veterinarily acceptable salt, hydrate or solvate thereof, and a therapeutically inert carrier.

13. A compound as defined in claim 1, for use in the amelioration, prevention and/or treatment of a disease caused by or related to delipidation of a neural tissue, preferably by inhibiting the expression and/or activity of the enzyme Carnitin-Palmitoyl-Transferase-1 (CPT-1).

14. The compound for use of claim 13, wherein the disease caused by or related to delipidation of a neural tissue is Morbus Alzheimer, Morbus Parkinson, amyotrophic lateral sclerosis (ALS), inflammatory diseases, acute traumatic events such as surgery or injury, AIDS related wasting due to the toxicity of reverse transcriptase inhibitors, mitochondrial myopathies, senescence and ageing, neuronal ischemia, a polyglutamine disease, dystonia, Leber's heredity optic neuropathy (LHON), schizophrenia, stroke, myodegenerative disorders, Mitochondrial Encephalomyopathy Lactic Acidosis and Strokelike Episodes (MELAS), Myoclonic Epilepsy associated with Ragged-Red Fibers (MERRF), Neuropathy, Ataxia, and Retinitis Pigmentosa (NARP), Progressive External Ophthalmoplegia (PEO), Leigh's disease, Kearns-Sayres Syndromes, muscular dystrophy, myotonic distrophy, chronic fatigue syndrome, Friedreich's Ataxia; developmental delay in cognitive, motor, language, executive function or social skills; epilepsy, peripheral neuropathy, optic neuropathy, autonomic neuropathy, neurogenic bowel dysfunction, sensorineural deafness, neurogenic bladder dysfunction, migraine; renal tubular acidosis, hepatic failure, lactic acidemia, parodontosis, Duchenne muscular dystrophy, Becker's muscular dystrophy, McArdle's disease, abnormities of the testosterone synthesis and/or hypoparathyroidism.

15. The compound for use of claim 13, wherein the disease caused by or related to delipidation of a neural tissue is amyotrophic lateral sclerosis (ALS).
